(19) United States
(12) Patent Application Publication
(10) Pub. No.: US 2010/0227880 A1
(43) Pub. Date: Sep. 9, 2010

(54) CHEMICAL COMPOUNDS

(76) Inventors: Kristjan Gudmundsson, Durham, NC (US); Elizabeth Madalena Turner, Durham, NC (US)

Correspondence Address: GLAXOSMITHKLINE;CORPORATE INTELLECTUAL PROPERTY, MAI B482, FIVE MOORE DR., PO BOX 13398, RESEARCH TRIANGLE PARK, NC, 27709-3398, US

(21) Appl. No.: 12/161,800

(22) PCT Filed: Jan. 24, 2007
(86) PCT No.: PCT/US07/60938

§ 371 Date: Jul. 23, 2008

Related U.S. Application Data
(60) Provisional application No. 60/761,883, filed on Jan. 25, 2006.

(51) Int. Cl. A61K 31/437; C07D 471/02

(52) U.S. Cl. 514/300; 546/121

(57) ABSTRACT

The present invention provides compounds of formula (I):

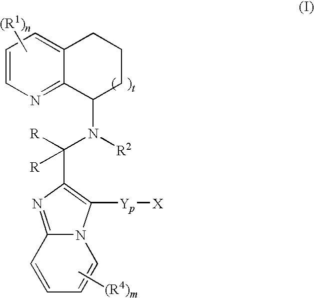

including salts, solvates, and pharmaceutically acceptable derivatives thereof, pharmaceutical formulations containing them, processes for their preparation, and methods of treatment using them.

### FIELD OF THE INVENTION

The present invention provides compounds that demonstrate protective effects on target cells from HIV infection in a manner as to bind to a chemokine receptor, and which affect the binding of the natural ligand or chemokine to a receptor such as CXCR4 of a target cell.

### BACKGROUND OF THE INVENTION

HIV gains entry into host cells by means of the CD4 receptor and at least one co-receptor expressed on the surface of the cell membrane. M-tropic strains of HIV utilize the chemokine receptor CCR5, whereas T-tropic strains of HIV mainly use CXCR4 as the co-receptor. HIV co-receptor usage largely depends on hyper-variable regions of the V3 loop located on the viral envelope protein gp120. Binding of gp120 with CD4 and the appropriate co-receptor results in a conformational change and unmasking of a second viral envelope protein called gp41. The protein gp41 subsequently interacts with the host cell membrane resulting in fusion of the viral envelop with the cell. Subsequent transfer of viral genetic information into the host cell allows for the continuation of viral replication. Thus infection of host cells with HIV is usually associated with the virus gaining entry into the cell via the formation of the ternary complex of CCR5 or CXCR4, CD4, and gp120.

A pharmacological agent that would inhibit the interaction of gp120 with either CCR5/CD4 or CXCR4/CD4 would be a useful therapeutic in the treatment of a disease, disorder, or condition characterized by infection with M-tropic or T-tropic strains, respectively, either alone or in combination therapy.

Evidence that administration of a selective CXCR4 antagonist could result in an effective therapy comes from in vitro studies that have demonstrated that addition of ligands selective for CXCR4 as well as CXCR4-neutralizing antibodies to cells can block HIV viral/host cell fusion. In addition, human studies with a selective CXCR4 antagonist, have demonstrated that such compounds can significantly reduce T-tropic HIV viral load in those patients that are either dual tropic or those where only the T-tropic form of the virus is present.

In addition to serving as a co-factor for HIV entry, it has been recently suggested that the direct interaction of the HIV viral protein gp120 with CXCR4 could be a possible cause of CD8⁺ T-cell apoptosis and AIDS-related dementia via induction of neuronal cell apoptosis.

The signal provided by SDF-1 on binding to CXCR4 may also play an important role in tumor cell proliferation and regulation of angiogenesis associated with tumor growth; the known angiogenic growth factors VEG-F and bFGF up-regulate levels of CXCR4 in endothelial cells and SDF-1 can induce neovascularization in vivo. In addition, leukemia cells that express CXCR4 migrate and adhere to lymph nodes and bone marrow stromal cells that express SDF-1.

The binding of SDF-1 to CXCR4 has also been implicated in the pathogenesis of atherosclerosis, renal allograft rejection asthma and allergic airway inflammation, Alzheimer's disease, and arthritis.

Additionally, CXCR4 antagonists may have a role in remodeling and repair of cardiac tissue and preserving cardiac function post myocardial infarction. After myocardial infarction, peripheral and bone marrow derived endothethial progenitor cells are found within the myocardium. It is thought that these cells result in improved ventricular function. This may be due to the production of cytokines that restore function and vascularization or to differentiation of the cells into functional myocardium. CXCL12 and CXCR4 are required for the homing of these stem cells to the myorcardium. In a preclinical study, a CXCR4 antagonist preserved chronic left ventricular function in rats after induction of a myocardial infarction by promoting mobilization and incorporation of bone marrow-derived enothethial progenitor cells into sites of myocardial neovascularization.

The present invention is directed to compounds that can act as agents that modulate chemokine receptor activity. Such chemokine receptors may include, but are not limited to, CCR1, CCR2, CCR3, CCR4, CCR5, CCR6, CCR7, CCR8, CXCR1, CXCR2, CXCR3, CXCR4, and CXCR5.

The present invention provides compounds that demonstrate protective effects on target cells from HIV infection in a manner as to bind to a chemokine receptor, and which affect the binding of the natural ligand or chemokine to a receptor, such as CXCR4 of a target cell.

### SUMMARY OF THE INVENTION

The present invention includes compounds of formula (I):

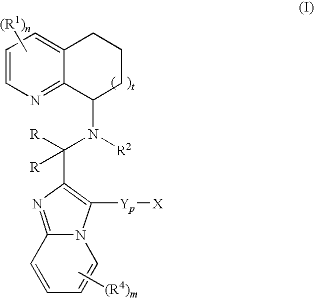

wherein:
t is 0, 1, or 2;
each R independently is H, C₁-C₈ alkyl, C₂-C₆ alkenyl, C₂-C₆ alkynyl, C₁-C₈ haloalkyl, C₃-C₈ cycloalkyl, —R^{a}Ay, —R^{a}OR¹⁰, —R^{a}N(R¹⁰)₂ or —R^{a}S(O)_{q}R¹⁰;
each R¹ independently is halogen, C₁-C₈ haloalkyl, C₁-C₈ alkyl, C₂-C₆ alkenyl, C₂-C₆ alkynyl, C₃-C₈ cycloalkyl, C₃-C₈ cycloalkenyl, -Ay, —N(H)Ay, -Het, —N(H)Het, —OR¹⁰, —OHet, —R^{a}OR¹⁰, —N(R⁶)R⁷, —R^{a}N(R⁶)R⁷, —R^{a}C(O)R¹⁰, —C(O)R¹⁰, —CO₂R¹⁰, —R^{a}CO₂R¹⁰, —C(O)N(R⁶)R⁷, —C(O)Ay, —C(O)Het, —S(O)₂N(R⁶)R⁷, —S(O)_{q}R¹⁰, cyano, nitro, or azido;
n is 0, 1, or 2;
R² is selected from a group consisting of H, C₁-C₈ alkyl, C₁-C₈ haloalkyl, C₃-C₈ cycloalkyl, C₂-C₆ alkenyl, C₂-C₆ alkynyl, —R^{a}cycloalkyl, —R^{a}Ay, —R^{a}OR¹⁰, or —R^{a}S(O)_{q}R¹⁰, wherein R² is not amine or alkylamine, or substituted with amine or alkylamine;
p is 0 or 1;
Y is —NR¹⁰, —O—, —C(O)N(R¹⁰)—, —N(R¹⁰)C(O)—, —C(O)—, —C(O)O—, —NR¹⁰C(O)N(R¹⁰)—, —S(O)_{q}—, —S(O)_{q}N(R¹⁰)—, or —N(R¹⁰)S(O)_{q}—;
X is —R^{a}N(R⁶)R⁷, -Ay[N(R⁶)R⁷]_{W}, —R^{a}Ay[N(R⁶)R⁷]_{w}, -Ay[R^{a}N(R⁶)R⁷]_{w}, —R^{a}Ay[R^{a}N(R⁶)R⁷]_{w}, -Het, —R^{a}Het, Het[N(R⁶)R⁷]_{w}, —R^{a}Het[N(R⁶)R⁷]_{w}, -Het[R^{a}N(R⁶)R⁷]_{w}, —R^{a}Het[R^{a}N(R⁶)R⁷]_{w}, -HetR^{a}Ay, or -HetR^{a}Het provided that when p is 0, m is 1 or 2, and X is —R^{a}N(R⁶)R⁷, then R^{a} is not methylene (—CH₂—);
each R^{a} independently is C₁-C₈ alkylene, C₃-C₈ cycloalkylene, C₂-C₆ alkenylene, C₃-C₈ cycloalkenylene, or C₂-C₆ alkynylene, optionally substituted with one or more C₁-C₈ alkyl, hydroxyl or oxo;
each R⁴ independently is halogen, C₁-C₈ haloalkyl, C₁-C₈ alkyl, C₂-C₆ alkenyl, C₂-C₆ alkynyl, C₃-C₈ cycloalkyl, C₃-C₈cycloalkenyl, -Ay, —NHAy, -Het, —NHHet, —OR¹⁰, —OHet, —R^{a}OR¹⁰, —N(R⁶)R⁷, R^{a}N(R⁶)R⁷, —R^{a}C(O)R¹⁰, —C(O)R¹⁰, —CO₂R¹⁰, —R^{a}CO₂R¹⁰, —C(O)N(R⁶)R⁷, —C(O)Ay, —C(O)Het, —S(O)₂N(R⁶)R⁷, —S(O)_{q}R¹⁰, —S(O)_{q}Het, cyano, nitro, or azido;
m is 0, 1, or 2;
each of R⁶ and R⁷ independently are selected from H, C₁-C₈ alkyl, C₁-C₈ alkenyl, C₂-C₆ alkynyl, C₃-C₈ cycloalkyl, C₃-C₈ cycloalkenyl, —R^{a}cycloalkyl, —R^{a}OR¹⁰, —R^{a}N(R⁸)R⁹, -Ay, -Het, —R^{a}Ay or —R^{a}Het;
each of R⁸ and R⁹ independently are selected from H or C₁-C₈ alkyl;
each R¹⁰ independently is H, C₁-C₈ alkyl, C₃-C₈ cycloalkyl, C₂-C₆ alkenyl, C₂-C₆ alkynyl, or -Ay;
each q independently is 0, 1, or 2;
each w independently is 1 or 2;
each Ay independently represents a C₃-C₁₀ aryl group optionally substituted with one or more of C₁-C₈ alkyl, C₂-C₆ alkenyl, C₂-C₆ alkynyl, C₁-C₈ alkoxy, hydroxyl, halogen, C₁-C₈ haloalkyl, C₃-C₈ cycloalkyl, C₃-C₈ cycloalkoxy, cyano, amide, amino, and C₁-Cₛ alkylamino; and
each Het independently represents a C₃-C₇ heterocyclyl or heteroaryl group optionally substituted with one or more of C₁-C₈ alkyl, C₂-C₆ alkenyl, C₂-C₆ alkynyl, C₁-C₈alkoxy, hydroxyl, halogen, C₁-C₈ haloalkyl, C₃-C₈ cycloalkyl, C₃-C₈ cycloalkoxy, cyano, amide, amino, and C₁-C₈ alkylamino; or pharmaceutically acceptable derivatives thereof.

The present invention features a compound of formula (I) wherein, t is 1 or 2 and all other substituents are as defined above or a pharmaceutically acceptable derivative thereof. The present invention features a compound of formula (I) wherein t is 1 and all other substituents are as defined above or a pharmaceutically acceptable derivative thereof.

The present invention features a compound of formula (I) wherein each R is H or alkyl and all other substituents are as defined above or a pharmaceutically acceptable derivative thereof. The present invention also features a compound of formula (I) wherein each R is H.

The present invention features a compound of formula (I) wherein n is 0 and all other substituents are as defined above or a pharmaceutically acceptable derivative thereof.

The present invention features a compound of formula (I) wherein n is 1 and R¹ is halogen, C₁-C₈ haloalkyl, C₁-C₈ alkyl, OR¹⁰, N(R⁶)R⁷, CO₂R¹⁰, C(O)N(R⁶)R⁷, or cyano and all other substituents are as defined above or a pharmaceutically acceptable derivative thereof.

The present invention features a compound of formula (I) wherein R² is H, C₁-C₈alkyl, C₁-C₈haloalkyl, R^{a}OR¹⁰, —R^{a}cycloalkyl or C₃-C₈cycloalkyl and all other substituents are as defined above or a pharmaceutically acceptable derivative thereof. The present invention features a compound of formula (I) wherein R² is C₁-C₈alkyl, —R^{a}cycloalkyl or C₃-C₈cycloalkyl. The present invention features a compound of formula (I) wherein R² is C₁-C₈alkyl.

The present invention features a compound of formula (I) wherein m is 0 and all other substituents are as defined above or a pharmaceutically acceptable derivative thereof.

The present invention features a compound of formula (I) wherein m is 1 or 2 and R⁴ is one or more of halogen, C₁-C₈haloalkyl, C₁-C₈alkyl, OR¹⁰, N(R⁶)R⁷, CO₂R¹⁰, C(O)N(R⁶)R⁷, or cyano and all other substituents are as defined above or a pharmaceutically acceptable derivative thereof. The present invention features a compound of formula (I) wherein m is 1 and all other substituents are as defined above or a pharmaceutically acceptable derivative thereof.

The present invention features a compound where m is 1 and R⁴ is —OR¹⁰, Het, —N(H)Het, —OHet, or —R^{a}N(R⁶)R⁷.

The present invention features a compound where m is 1 or 2 and X is —R^{a}N(R⁶)R⁷, -Ay[N(R⁶)R⁷]_{w}, -Ay[R^{a}N(R⁶)R⁷]_{w}, —R^{a}Ay[R^{a}N(R⁶)R⁷]_{w}, -Het, —R^{a}Het, Het[N(R⁶)R⁷]_{w}, —R^{a}Het[N(R⁶)R⁷]_{w}, -Het[R^{a}N(R⁶)R⁷]_{w}, —R^{a}Het[R^{a}N(R⁶)R⁷]_{w}, -HetR^{a}Ay, or -HetR^{a}Het provided that when p is 0, m is 1 or 2, and X is —R^{a}N(R⁶)R⁷, then R^{a} is not methylene (—CH₂—) and all other substituents are as defined above or a pharmaceutically acceptable derivative thereof.

The present invention features a compound of formula (I) wherein R⁴ is one or more of halogen, C₁-C₈haloalkyl, C₁-C₈alkyl, —OR¹⁰, —CO₂R¹⁰, —C(O)N(R⁶)R⁷, or cyano and all other substituents are as defined above or a pharmaceutically acceptable derivative thereof.

The present invention features a compound of formula (I) wherein Het is piperidine, piperazine, azetidine, pyrrolidine, imidazole, pyridine, and the like and all other substituents are as defined above or a pharmaceutically acceptable derivative thereof.

The present invention features a compound of formula (I) wherein, each R is H and t is 1 and all other substituents are as defined above or a pharmaceutically acceptable derivative thereof.

The present invention features a compound of formula (I) wherein m is 1 and the substituent R⁴ is located on the depicted imidazopyridine ring as in formula (I-A):

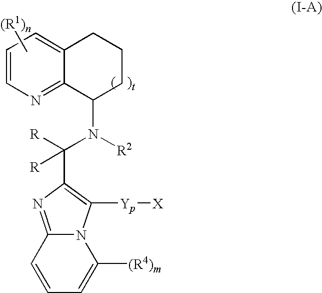

wherein all substituents are as defined above with respect to formula (I), or pharmaceutically acceptable derivatives thereof.

One aspect of the invention includes compounds of formula (I-A) wherein R⁴ is —OR¹⁰, Het, —N(H)Het, —OHet, —R^{a}N(R⁶)R⁷, or pharmaceutically acceptable derivatives thereof.

One aspect of the invention includes of formula (I-A) wherein R⁴ is C₁-C₈alkyl, OR¹⁰ or N(R⁶)R⁷.

The present invention features a compound of formula (I) wherein p is 0, m is 0 and X is —R^{a}N(R⁶)R⁷, -Ay[R^{a}N(R⁶)R⁷]_{w}, -Het, —R^{a}Het, Het[N(R⁶)R⁷]_{w}, —R^{a}Het[N(R⁶)R⁷]_{w}, -Het[R^{a}N(R⁶)R⁷]_{w}, —R^{a}Het[R^{a}N(R⁶)R⁷]_{w}; and all other substituents are as defined above or a pharmaceutically acceptable derivative thereof.

The present invention features a compound of formula (I) wherein m is 0 and X is —R^{a}N(R⁶)R⁷, -Het, —R^{a}Het, Het[N(R⁶)R⁷]_{w}, or —R^{a}Het[N(R⁶)R⁷]_{w}; and all other substituents are as defined above or a pharmaceutically acceptable derivative thereof. The present invention features a compound of formula (I) wherein m is 0 and X is —R^{a}N(R⁶)R⁷, -Het, or —R^{a}Het; and all other substituents are as defined above or a pharmaceutically acceptable derivative thereof.

The present invention features a compound of formula (I) wherein p is 1; Y is —NR¹⁰—, —O—, —S—, —C(O)NR¹⁰—, —N(R¹⁰)CO—, or —S(O)_{q}N(R¹⁰)—; and X is —R^{a}(N(R⁶)R⁷), -Ay[N(R⁶)R⁷]_{W}, —R^{a}Ay[N(R⁶)R⁷]_{w}, -Ay[R^{a}N(R⁶)R⁷]_{w}, —R^{a}Ay[R^{a}N(R⁶)R⁷]_{w}, -Het, —R^{a}Het, Het[N(R⁶)R⁷]_{w}, —R^{a}Het[N(R⁶)R⁷]_{w}, -Het[R^{a}N(R⁶)R⁷]_{w}, —R^{a}Het[R^{a}N(R⁶)R⁷]_{w}, -HetR^{a}Ay, or -HetR^{a}Het; and all other substituents are as defined above or a pharmaceutically acceptable derivative thereof. The present invention features a compound of formula (I) wherein p is 1, Y is —NR¹⁰—, —O—, —C(O)N(R¹⁰)—, —N(R¹⁰)CO— and X is —R^{a}N(R⁶)R⁷, -Het, —R^{a}Het, Het[N(R⁶)R⁷]_{w}; and all other substituents are as defined above or a pharmaceutically acceptable derivative thereof.

One aspect of the invention includes compounds of formula (I-A) where t is 1 or 2; R is H or C₁-C₈ alkyl; R² is C₁-C₈ alkyl, C₃-C₈ cycloalkyl, or —R^{a}cycloalkyl; n is 0; m is 0; p is 0 and X is —R^{a}N(R⁶)R⁷, -Het, —R^{a}Het, or Het[N(R⁶)R⁷]_{w}, —R⁶ and R⁷ is H or C₁-C₈ alkyl and -Het is unsubstituted or substituted with C₁-C₈ alkyl or C₃-C₈ cycloalkyl.

One aspect of the invention includes compounds of formula (I-A) where t is 1 or 2; R is H or C₁-C₈ alkyl; R² is C₁-C₈ alkyl, C₃-C₈ cycloalkyl, or —R^{a}cycloalkyl; n is 0; m is 1 and R⁴ is —OR¹⁰, —N(R⁶)R⁷, Het or N(H)Het; p is 0 and X is —R^{a}N(R⁶)R⁷, provided that R^{a} is not methylene, -Het, —R^{a}Het, or Het[N(R⁶)R⁷]_{w}, R⁶ and R⁷ is H or C₁-C₈ alkyl and -Het is unsubstituted or substituted with C₁-C₈ alkyl or C₃-C₈ cycloalkyl.

One aspect of the invention includes compounds of formula (I-A) where t is 1 or 2; R is H or C₁-C₈ alkyl; R² is C₁-C₈ alkyl, C₃-C₈ cycloalkyl, or —R^{a}cycloalkyl; n is 0; m is 0; p is 1; Y is —N(R¹⁰)—, —O—, —C(O)N(R¹⁰)—, or —N(R¹⁰)CO—; X is —R^{a}N(R⁶)R⁷, -Het, —R^{a}Het or Het[N(R⁶)R⁷]_{w}, and R⁶ and R⁷ is H or C₁-C₈ alkyl and Het is unsubstituted or substituted with C₁-C₈ alkyl or C₃-C₈ cycloalkyl.

One aspect of the invention includes compounds of formula (I-A) where t is 1 or 2; R is H or C₁-C₈ alkyl; R² is C₁-C₈ alkyl, C₃-C₈ cycloalkyl, or —R^{a}cycloalkyl; n is 0; m is 1 and R⁴ is —OR¹⁰, —N(R⁶)R⁷, Het or N(H)Het; p is 1; Y is —N(R¹⁰)—, —O—, —C(O)N(R¹⁰)—, or —N(R¹⁰)CO—; X is —R^{a}N(R⁶)R⁷ provided that R^{a} is not methylene, -Het, or —R^{a}Het and R⁶ and R⁷ is H or C₁-C₈ alkyl and Het is unsubstituted or substituted with C₁-C₈ alkyl or C₃-C₈ cycloalkyl.

Compounds of the present invention include:
(8S)-N-Methyl-N-{[3-(4-pyridinyl)imidazo[1,2-a]pyridin-2-yl]methyl}-5,6,7,8-tetrahydro-8-quinolinamine;
(8S)-N-{[3-(3-Aminopropyl)imidazo[1,2-a]pyridin-2-yl]methyl}-N-methyl-5,6,7,8-tetrahydro-8-quinolinamine;
(8S)-N-({3-[3-(Dimethylamino)propyl]imidazo[1,2-a]pyridin-2-yl}methyl)-N-methyl-5,6,7,8-tetrahydro-8-quinolinamine;
(8S)-N-Methyl-N-[(3-{3-[(2-methylpropyl)amino]propyl}imidazo[1,2-a]pyridin-2-yl)methyl]-5,6,7,8-tetrahydro-8-quinolinamine;
(8S)-N-({3-[(Dimethylamino)methyl]imidazo[1,2-a]pyridin-2-yl}methyl)-N-methyl-5,6,7,8-tetrahydro-8-quinolinamine;
N²,N²-Dimethyl-N¹-{[2-({methyl[(8S)-5,6,7,8-tetrahydro-8-quinolinyl]amino}methyl)imidazo[1,2-a]pyridin-3-yl]methyl}glycinamide;
(8S)-N-Methyl-N-{[3-(1-pyrrolidinylmethyl)imidazo[1,2-a]pyridin-2-yl]methyl}-5,6,7,8-tetrahydro-8-quinolinamine;
(8S)-N-({3-[6-(Dimethylamino)-3-pyridinyl]imidazo[1,2-a]pyridin-2-yl}methyl)-N-methyl-5,6,7,8-tetrahydro-8-quinolinamine;
(8S)-N-Methyl-N-{[3-(1-piperidinylmethyl)imidazo[1,2-a]pyridin-2-yl]methyl}-5,6,7,8-tetrahydro-8-quinolinamine;
(8S)-N-[(3-{[3-(Dimethylamino)-1-pyrrolidinyl]methyl}imidazo[1,2-a]pyridin-2-yl)methyl]-N-methyl-5,6,7,8-tetrahydro-8-quinolinamine;
(8S)-N-Methyl-N-({3-[6-(4-methyl-1-piperazinyl)-3-pyridinyl]imidazo[1,2-a]pyridin-2-yl}methyl)-5,6,7,8-tetrahydro-8-quinolinamine;
N,N,N′-Trimethyl-N′-{[2-({methyl[(8S)-5,6,7,8-tetrahydro-8-quinolinyl]amino}methyl)imidazo[1,2-a]pyridin-3-yl]methyl}-1,2-ethanediamine;
(8S)-N-Methyl-N-[(3-{[methyl(1-methylethyl)amino]methyl}imidazo[1,2-a]pyridin-2-yl)methyl]-5,6,7,8-tetrahydro-8-quinolinamine;
(8S)-N-Methyl-N-[(3-{[methyl(1-methyl-3-pyrrolidinyl)amino]methyl}imidazo[1,2-a]pyridin-2-yl)methyl]-5,6,7,8-tetrahydro-8-quinolinamine;
(8S)-N-Methyl-N-[(3-{[methyl(1-methyl-4-piperidinyl)amino]methyl}imidazo[1,2-a]pyridin-2-yl)methyl]-5,6,7,8-tetrahydro-8-quinolinamine;
(Methyl{[2-({methyl[(8S)-5,6,7,8-tetrahydro-8-quinolinyl]amino}methyl)imidazo[1,2-a]pyridin-3-yl]methyl}amino)acetonitrile;
3-(Methyl{[2-({methyl[(8S)-5,6,7,8-tetrahydro-8-quinolinyl]amino}methyl)imidazo[1,2-a]pyridin-3-yl]methyl}amino)propanenitrile;
(8S)-N-Methyl-N-{[3-(3-pyridinyl)imidazo[1,2-a]pyridin-2-yl]methyl}-5,6,7,8-tetrahydro-8-quinolinamine;
(8S)-N-Methyl-N-({3-[6-(4-morpholinyl)-3-pyridinyl]imidazo[1,2-a]pyridin-2-yl}methyl)-5,6,7,8-tetrahydro-8-quinolinamine;
(8S)-N-Methyl-N-{[3-(4-morpholinylmethyl)imidazo[1,2-a]pyridin-2-yl]methyl}-5,6,7,8-tetrahydro-8-quinolinamine;
(8S)-N-Methyl-N-({3-[6-(1-pyrrolidinyl)-3-pyridinyl]imidazo[1,2-a]pyridin-2-yl}methyl)-5,6,7,8-tetrahydro-8-quinolinamine;
(8S)-N-{[3-({Bis[2-(methyloxy)ethyl]amino}methyl)imidazo[1,2-a]pyridin-2-yl]methyl}-N-methyl-5,6,7,8-tetrahydro-8-quinolinamine;
(8S)-N-({3-[(Diethylamino)methyl]imidazo[1,2-a]pyridin-2-yl}methyl)-N-methyl-5,6,7,8-tetrahydro-8-quinolinamine;
(8S)-N-Methyl-N-{[3-({methyl[2-(methyloxy)ethyl]amino}methyl)imidazo[1,2-a]pyridin-2-yl)]ethyl}-5,6,7,8-tetrahydro-8-quinolinamine;
(8S)-N-Methyl-N-[(3-{[methyl(2-methylpropyl)amino]methyl}imidazo[1,2-a]pyridin-2-yl)methyl]-5,6,7,8-tetrahydro-8-quinolinamine;
(8S)-N-({3-[2-(Dimethylamino)-4-pyridinyl]imidazo[1,2-a]pyridin-2-yl}methyl)-N-methyl-5,6,7,8-tetrahydro-8-quinolinamine;
(8S)-N-Methyl-N-({3-[2-(4-morpholinyl)-4-pyridinyl]imidazo[1,2-a]pyridin-2-yl}methyl)-5,6,7,8-tetrahydro-8-quinolinamine;
N,N-dimethyl-N′-[(2-{[methyl(5,6,7,8-tetrahydro-8-quinolinyl)amino]methyl}imidazo[1,2-a]pyridin-3-yl)methyl]-1,2-ethanediamine;
N-methyl-N-({3-[(4-methyl-1-piperazinyl)methyl]imidazo[1,2-a]pyridin-2-yl}methyl)-5,6,7,8-tetrahydro-8-quinolinamine;
N-[(2-{[methyl(5,6,7,8-tetrahydro-8-quinolinyl)amino]methyl}imidazo[1,2-a]pyridin-3-yl)methyl]-1,3-propanediamine;
N-[(3-{[3-(dimethylamino)-1-pyrrolidinyl]carbonyl}imidazo[1,2-a]pyridin-2-yl)methyl]-N-methyl-5,6,7,8-tetrahydro-8-quinolinamine;
N,N,N′-trimethyl-N′-{5-[2-({methyl[(8S)-5,6,7,8-tetrahydro-8-quinolinyl]amino}methyl)imidazo[1,2-a]pyridin-3-yl]-2-pyridinyl}-1,2-ethanediamine;
(8S)-N-[(3-{6-[3-(Dimethylamino)-1-pyrrolidinyl]-3-pyridinyl}imidazo[1,2-a]pyridin-2-yl)methyl]-N-methyl-5,6,7,8-tetrahydro-8-quinolinamine;
N-[2-(dimethylamino)ethyl]-2-{[methyl(5,6,7,8-tetrahydro-8-quinolinyl)amino]methyl}imidazo[1,2-a]pyridine-3-carboxamide;
N-({3-[(3-amino-1-azetidinyl)carbonyl]imidazo[1,2-a]pyridin-2-yl}methyl)-N-methyl-5,6,7,8-tetrahydro-8-quinolinamine;
2-{[methyl(5,6,7,8-tetrahydro-8-quinolinyl)amino]methyl}-N-[2-(1-pyrrolidinyl)ethyl]imidazo[1,2-a]pyridine-3-carboxamide;
N-methyl-N-({3-[(4-methyl-1-piperazinyl)carbonyl]imidazo[1,2-a]pyridin-2-yl}methyl)-5,6,7,8-tetrahydro-8-quinolinamine;
N-methyl-N-[(3-{[4-(1-methylethyl)-1-piperazinyl]carbonyl}imidazo[1,2-a]pyridin-2-yl)methyl]-5,6,7,8-tetrahydro-8-quinolinamine; and pharmaceutically acceptable derivatives thereof Compounds of the present invention further include:
(8S)-N-Methyl-N-{[3-(4-pyridinyl)imidazo[1,2-a]pyridin-2-yl]methyl}-5,6,7,8-tetrahydro-8-quinolinamine;
(8S)-N-{[3-(3-Aminopropyl)imidazo[1,2-a]pyridin-2-yl]methyl}-N-methyl-5,6,7,8-tetrahydro-8-quinolinamine;
(8S)-N-({3-[3-(Dimethylamino)propyl]imidazo[1,2-a]pyridin-2-yl}methyl)-N-methyl-5,6,7,8-tetrahydro-8-quinolinamine;
(8S)-N-Methyl-N-[(3-{3-[(2-methylpropyl)amino]propyl}imidazo[1,2-a]pyridin-2-yl)methyl]-5,6,7,8-tetrahydro-8-quinolinamine;
(8S)-N-({3-[(Dimethylamino)methyl]imidazo[1,2-a]pyridin-2-yl}methyl)-N-methyl-5,6,7,8-tetrahydro-8-quinolinamine;
N²,N²-Dimethyl-N¹-{[2-({methyl[(8S)-5,6,7,8-tetrahydro-8-quinolinyl]amino}methyl)imidazo[1,2-a]pyridin-3-yl]methyl}glycinamide;
(8S)-N-Methyl-N-{[3-(1-pyrrolidinylmethyl)imidazo[1,2-a]pyridin-2-yl]methyl}-5,6,7,8-tetrahydro-8-quinolinamine;
(8S)-N-({3-[6-(Dimethylamino)-3-pyridinyl]imidazo[1,2-a]pyridin-2-yl}methyl)-N-methyl-5,6,7,8-tetrahydro-8-quinolinamine;
(8S)-N-Methyl-N-{[3-(1-piperidinylmethyl)imidazo[1,2-a]pyridin-2-yl]methyl}-5,6,7,8-tetrahydro-8-quinolinamine;
(8S)-N-[(3-{[3-(Dimethylamino)-1-pyrrolidinyl]methyl}imidazo[1,2-a]pyridin-2-yl)methyl]-N-methyl-5,6,7,8-tetrahydro-8-quinolinamine;
(8S)-N-Methyl-N-({3-[6-(4-methyl-1-piperazinyl)-3-pyridinyl]imidazo[1,2-a]pyridin-2-yl}methyl)-5,6,7,8-tetrahydro-8-quinolinamine;
N,N,N′-Trimethyl-N′-{[2-({methyl[(8S)-5,6,7,8-tetrahydro-8-quinolinyl]amino}methyl)imidazo[1,2-a]pyridin-3-yl]methyl}-1,2-ethanediamine;
(8S)-N-Methyl-N-[(3-{[methyl(1-methylethyl)amino]methyl}imidazo[1,2-a]pyridin-2-yl)methyl]-5,6,7,8-tetrahydro-8-quinolinamine;
(8S)-N-Methyl-N-[(3-{[methyl(1-methyl-3-pyrrolidinyl)amino]methyl}imidazo[1,2-a]pyridin-2-yl)methyl]-5,6,7,8-tetrahydro-8-quinolinamine;
(8S)-N-Methyl-N-[(3-{[methyl(1-methyl-4-piperidinyl)amino]methyl}imidazo[1,2-a]pyridin-2-yl)methyl]-5,6,7,8-tetrahydro-8-quinolinamine;
(Methyl{[2-({methyl[(8S)-5,6,7,8-tetrahydro-8-quinolinyl]amino}methyl)imidazo[1,2-a]pyridin-3-yl]methyl}amino)acetonitrile;
3-(Methyl{[2-({methyl[(8S)-5,6,7,8-tetrahydro-8-quinolinyl]amino}methyl)imidazo[1,2-a]pyridin-3-yl]methyl}amino)propanenitrile; and pharmaceutically acceptable derivatives thereof.

One aspect of the present invention includes the compounds substantially as hereinbefore defined with reference to any one of the Examples.

One aspect of the present invention includes a pharmaceutical composition comprising one or more compounds of the present invention and a pharmaceutically acceptable carrier.

One aspect of the present invention includes one or more compounds of the present invention for use as an active therapeutic substance.

One aspect of the present invention includes one or more compounds of the present invention for use in the treatment (including prophylaxis) of diseases and conditions caused by inappropriate activity of CXCR4.

One aspect of the present invention includes one or more compounds of the present invention for use in the treatment (including prophylaxis) of HIV infection, myocardial infarction, diseases associated with hematopoiesis, controlling the side effects of chemotherapy, enhancing the success of bone marrow transplantation, enhancing wound healing and burn treatment, combating bacterial infections in leukemia, inflammation, inflammatory or allergic diseases, asthma, allergic rhinitis, hypersensitivity lung diseases, hypersensitivity pneumonitis, eosinophilic pneumonitis, delayed-type hypersensitivity, interstitial lung disease (ILD), idiopathic pulmonary fibrosis, systemic lupus erythematosus, ankylosing spondylitis, systemic sclerosis, Sjogren's syndrome, polymyositis or dermatomyositis, systemic anaphylaxis or hypersensitivity responses, drug allergies, insect sting allergies, autoimmune diseases, rheumatoid arthritis, psoriatic arthritis, systemic lupus erythematosus, myastenia gravis, juvenile onset diabetes, glomerulonephritis, autoimmune throiditis, graft rejection, allograft rejection, graft-versus-host disease, inflammatory bowel diseases, Crohn's disease, ulcerative colitus, spondylo-arthropathies, scleroderma, psoriasis, T-cell-mediated psoriasis, inflammatory dermatoses, dermatitis, eczema, atopic dermatitis, allergic contact dermatitis, urticaria, vasculitis, necrotizing, cutaneous, hypersensitivity vasculitis, eoosinophilic myotis, eosinophilic fasciitis, and brain, breast, prostate, lung, or haematopoetic tissue cancers. In one embodiment the condition or disease is HIV infection, rheumatoid arthritis, inflammation, or cancer. In yet another embodiment the disease is HIV infection.

One aspect of the present invention includes the use of one or more compounds of the present invention in the manufacture of a medicament for use in the treatment (including prophylaxis) of a condition or disease modulated by a chemokine receptor. Preferably the chemokine receptor is CXCR4.

One aspect of the present invention includes use of one or more compounds of the present invention in the manufacture of a medicament for use in the treatment (including prophylaxis) of HIV infection, myocardial infarction, diseases associated with hematopoiesis, controlling the side effects of chemotherapy, enhancing the success of bone marrow transplantation, enhancing wound healing and burn treatment, combating bacterial infections in leukemia, inflammation, inflammatory or allergic diseases, asthma, allergic rhinitis, hypersensitivity lung diseases, hypersensitivity pneumonitis, eosinophilic pneumonitis, delayed-type hypersensitivity, interstitial lung disease (ILD), idiopathic pulmonary fibrosis, systemic lupus erythematosus, ankylosing spondylitis, systemic sclerosis, Sjogren's syndrome, polymyositis or dermatomyositis, systemic anaphylaxis or hypersensitivity responses, drug allergies, insect sting allergies, autoimmune diseases, rheumatoid arthritis, psoriatic arthritis, systemic lupus erythematosus, myastenia gravis, juvenile onset diabetes, glomerulonephritis, autoimmune throiditis, graft rejection, allograft rejection, graft-versus-host disease, inflammatory bowel diseases, Crohn's disease, ulcerative colitus, spondylo-arthropathies, scleroderma, psoriasis, T-cell-mediated psoriasis, inflammatory dermatoses, dermatitis, eczema, atopic dermatitis, allergic contact dermatitis, urticaria, vasculitis, necrotizing, cutaneous, hypersensitivity vasculitis, eoosinophilic myotis, eosinophilic fasciitis, and brain, breast, prostate, lung, or haematopoetic tissue cancers. Preferably the use relates to a medicament wherein the condition or disorder is HIV infection, rheumatoid arthritis, inflammation, or cancer.

One aspect of the present invention includes a method for the treatment (including prophylaxis) of a condition or disease modulated by a chemokine receptor comprising the administration of one or more compounds of the present invention. Preferably the chemokine receptor is CXCR4.

One aspect of the present invention includes a method for the treatment (including prophylaxis) of HIV infection, myocardial infarction, diseases associated with hematopoiesis, controlling the side effects of chemotherapy, enhancing the success of bone marrow transplantation, enhancing wound healing and burn treatment, combating bacterial infections in leukemia, inflammation, inflammatory or allergic diseases, asthma, allergic rhinitis, hypersensitivity lung diseases, hypersensitivity pneumonitis, eosinophilic pneumonitis, delayed-type hypersensitivity, interstitial lung disease (ILD), idiopathic pulmonary fibrosis, systemic lupus erythematosus, ankylosing spondylitis, systemic sclerosis, Sjogren's syndrome, polymyositis or dermatomyositis, systemic anaphylaxis or hypersensitivity responses, drug allergies, insect sting allergies, autoimmune diseases, rheumatoid arthritis, psoriatic arthritis, systemic lupus erythematosus, myastenia gravis, juvenile onset diabetes, glomerulonephritis, autoimmune throiditis, graft rejection, allograft rejection, graft-versus-host disease, inflammatory bowel diseases, Crohn's disease, ulcerative colitus, spondylo-arthropathies, scleroderma, psoriasis, T-cell-mediated psoriasis, inflammatory dermatoses, dermatitis, eczema, atopic dermatitis, allergic contact dermatitis, urticaria, vasculitis, necrotizing, cutaneous, hypersensitivity vasculitis, eoosinophilic myotis, eosinophilic fasciitis, and brain, breast, prostate, lung, or haematopoetic tissue cancers comprising the administration of one or more compounds of the present invention.

One aspect of the present invention includes a method for the treatment (including prophylaxis) of HIV infection, rheumatoid arthritis, inflammation, or cancer comprising the administration of one or more compounds of the present invention. One aspect of the invention includes a method for the treatment (including prophylaxis) of HIV infection.

### DETAILED DESCRIPTION OF THE INVENTION

Terms are used within their accepted meanings. The following definitions are meant to clarify, but not limit, the terms defined.

As used herein the term “alkyl” alone or in combination with any other term, refers to a straight or branched chain hydrocarbon, containing from one to twelve carbon atoms, unless specified otherwise. Examples of “alkyl” as used herein include, but are not limited to, methyl, ethyl, propyl, isopropyl, isobutyl, n-butyl, tert-butyl, sec-butyl, isopentyl, n-pentyl, n-hexyl, and the like.

As used throughout this specification, the preferred number of atoms, such as carbon atoms, will be represented by, for example, the phrase “Cₓ—C_{y} alkyl,” which refers to an alkyl group, as herein defined, containing the specified number of carbon atoms. Similar terminology will apply for other preferred terms and ranges as well.

As used herein the term “alkenyl” refers to a straight or branched chain aliphatic hydrocarbon containing one or more carbon-to-carbon double bonds. Examples include, but are not limited to, vinyl, allyl, and the like.

As used herein the term “alkynyl” refers to a straight or branched chain aliphatic hydrocarbon containing one or more carbon-to-carbon triple bonds, which may occur at any stable point along the chain. Examples include, but are not limited to, ethynyl, propynyl, butynyl, pentynyl, and the like.

As used herein, the term “alkylene” refers to an optionally substituted straight or branched chain divalent hydrocarbon radical, preferably having from one to ten carbon atoms, unless specified otherwise. Examples of “alkylene” as used herein include, but are not limited to, methylene, ethylene, n-propylene, n-butylene, and the like. Preferred substituent groups include C₁-C₈ alkyl, hydroxyl or oxo.

As used herein, the term “alkenylene” refers to a straight or branched chain divalent hydrocarbon radical, preferably having from two to ten carbon atoms, unless specified otherwise, containing one or more carbon-to-carbon double bonds. Examples include, but are not limited to, vinylene, allylene or 2-propenylene, and the like.

As used herein, the term “alkynylene” refers to a straight or branched chain divalent hydrocarbon radical, preferably having from two to ten carbon atoms, unless otherwise specified, containing one or more carbon-to-carbon triple bonds. Examples include, but are not limited to, ethynylene and the like.

As used herein, the term “cycloalkyl” refers to an optionally substituted non-aromatic cyclic hydrocarbon ring. Unless otherwise indicated, cycloalkyl is composed of three to eight carbon atoms, Exemplary “cycloalkyl” groups include, but are not limited to, cyclopropyl, cyclobutyl, cyclopentyl, cyclohexyl, and cycloheptyl. As used herein, the term “cycloalkyl” includes an optionally substituted fused polycyclic hydrocarbon saturated ring and aromatic ring system, namely polycyclic hydrocarbons with less than maximum number of non-cumulative double bonds, for example where a saturated hydrocarbon ring (such as a cyclopentyl ring) is fused with an aromatic ring (herein “aryl,” such as a benzene ring) to form, for example, groups such as indane. Preferred substituent groups include C₁-C₈ alkyl, C₂-C₆ alkenyl, C₂-C₆ alkynyl, C₁-C₈ alkoxy, hydroxyl, halogen, C₁-C₈ haloalkyl, C₃-C₈ cycloalkyl, C₃-C₈ cycloalkoxy, cyano, amide, amino, and C₁-C₈ alkylamino.

As used herein, the term “cycloalkenyl” refers to an optionally substituted non-aromatic cyclic hydrocarbon ring containing one or more carbon-to-carbon double bonds which optionally includes an alkylene linker through which the cycloalkenyl may be attached. Exemplary “cycloalkenyl” groups include, but are not limited to, cyclopropenyl, cyclobutenyl, cyclopentenyl, cyclohexenyl, and cycloheptenyl. Preferred substituent groups include C₁-C₈ alkyl, C₂-C₆ alkenyl, C₂-C₆ alkynyl, C₁-C₈ alkoxy, hydroxyl, halogen, C₁-C₈ haloalkyl, C₃-C₈ cycloalkyl, C₃-C₈ cycloalkoxy, cyano, amide, amino, and C₁-C₈ alkylamino.

As used herein, the term “cycloalkylene” refers to a divalent, optionally substituted non-aromatic cyclic hydrocarbon ring. Exemplary “cycloalkylene” groups include, but are not limited to, cyclopropylene, cyclobutylene, cyclopentylene, cyclohexylene, and cycloheptylene. Preferred substituent groups include C₁-C₈ alkyl, C₂-C₆ alkenyl, C₂-C₆ alkynyl, C₁-C₈ alkoxy, hydroxyl, halogen, C₁-C₈ haloalkyl, C₃-C₈ cycloalkyl, C₃-C₈ cycloalkoxy, cyano, amide, amino, and C₁-C₈ alkylamino.

As used herein, the term “cycloalkenylene” refers to a divalent optionally substituted non-aromatic cyclic hydrocarbon ring containing one or more carbon-to-carbon double bonds. Exemplary “cycloalkenylene” groups include, but are not limited to, cyclopropenylene, cyclobutenylene, cyclopentenylene, cyclohexenylene, and cycloheptenylene. Preferred substituent groups include C₁-C₈ alkyl, C₂-C₆ alkenyl, C₂-C₆ alkynyl, C₁-C₈ alkoxy, hydroxyl, halogen, C₁-C₈ haloalkyl, C₃-C₈ cycloalkyl, C₃-C₈ cycloalkoxy, cyano, amide, amino, and C₁-C₈ alkylamino.

As used herein, the term “heterocycle”, “heterocyclic” or “heterocyclyl” refers to an optionally substituted mono- or polycyclic ring system containing one or more degrees of unsaturation and also containing one or more heteroatoms. Preferred heteroatoms include N, O, and/or S, including N-oxides, sulfur oxides, and dioxides. More preferably, the heteroatom is N.

Preferably the heterocyclyl ring is three to twelve-membered, unless otherwise indicated, and is either fully saturated or has one or more degrees of unsaturation. Such rings may be optionally fused to one or more of another “heterocyclic” ring(s) or cycloalkyl ring(s). Examples of “heterocyclic” groups include, but are not limited to, tetrahydrofuran, pyran, 1,4-dioxane, 1,3-dioxane, piperidine, piperazine, pyrrolidine, morpholine, tetrahydrothiopyran, aziridine, azetidine and tetrahydrothiophene. When the heterocyclic ring has substituents, it is understood that the substituents may be attached to any atom in the ring, whether a heteroatom or a carbon atom, provided that a stable chemical structure results. Preferred substituent groups include C₁-C₈ alkyl, C₂-C₆ alkenyl, C₂-C₈ alkynyl, C₁-C₈ alkoxy, hydroxyl, halogen, C₁-C₈ haloalkyl, C₃-C₈ cycloalkyl, C₃-C₈ cycloalkoxy, cyano, amide, amino, and C₁-C₈ alkylamino.

As used herein, the term “aryl” refers to an optionally substituted carbocyclic aromatic moiety (such as phenyl or naphthyl) containing the specified number of carbon atoms, preferably 6-14 carbon atoms or 6-10 carbon atoms. The term aryl also refers to optionally substituted ring systems, for example anthracene, phenanthrene, or naphthalene ring systems. Examples of “aryl” groups include, but are not limited to, phenyl, naphthyl, indenyl, azulenyl, fluorenyl, anthracenyl, phenanthrenyl, tetrahydronaphthyl, indanyl, phenathridinyl, and the like. Unless otherwise indicated, the term aryl also includes each possible positional isomer of an aromatic hydrocarbon radical, such as 1-naphthyl, 2-naphthyl, 5-tetrahydronaphthyl, 6-tetrahydronaphthyl, 1 phenanthridinyl, 2-phenanthridinyl, 3-phenanthridinyl, 4-phenanthridinyl, and the like. Preferred substituent groups include C₁-C₈ alkyl, C₂-C₆ alkenyl, C₂-C₆ alkynyl, C₁-C₈ alkoxy, hydroxyl, halogen, C₁-C₈ haloalkyl, C₃-C₈ cycloalkyl, C₃-C₈ cycloalkoxy, cyano, amide, amino, and C₁-C₈ alkylamino.

As used herein, the term “heteroaryl” refers to an optionally substituted monocyclic five to seven membered aromatic ring unless otherwise specified, or to an optionally substituted fused bicyclic aromatic ring system comprising two of such aromatic rings. These heteroaryl rings contain one or more nitrogen, sulfur, and/or oxygen atoms, where N-oxides, sulfur oxides, and dioxides are permissible heteroatom substitutions. Preferably, the heteroatom is N.

Examples of “heteroaryl” groups used herein include, but should not be limited to, furan, thiophene, pyrrole, imidazole, pyrazole, triazole, tetrazole, thiazole, oxazole, isoxazole, oxadiazole, thiadiazole, isothiazole, pyridine, pyridazine, pyrazine, pyrimidine, quinoline, isoquinoline, benzofuran, benzothiophene, indole, indazole, benzimidizolyl, imidazopyridinyl, pyrazolopyridinyl, and pyrazolopyrimidinyl. Preferred substituent groups include C₁-C₈ alkyl, C₂-C₆ alkenyl, C₂-C₆ alkynyl, C₁-C₈ alkoxy, hydroxyl, halogen, C₁-C₈ haloalkyl, C₃-C₈ cycloalkyl, C₃-C₈ cycloalkoxy, cyano, amide, amino, and alkylamino.

As used herein the term “halogen” refers to fluorine, chlorine, bromine, or iodine.

As used herein the term “haloalkyl” refers to an alkyl group, as defined herein, which is substituted with at least one halogen. Examples of branched or straight chained “haloalkyl” groups useful in the present invention include, but are not limited to, methyl, ethyl, propyl, isopropyl, n-butyl, and t-butyl substituted independently with one or more halogens, e.g., fluoro, chloro, bromo, and iodo. The term “haloalkyl” should be interpreted to include such substituents as perfluoroalkyl groups and the like.

As used herein the term “alkoxy” refers to a group —OR′, where R′ is alkyl as defined. Examples of suitable alkoxy radicals include, but are not limited to, methoxy, ethoxy, propoxy, isopropoxy, n-butoxy, isobutoxy, sec-butoxy, tert-butoxy, and the like.

As used herein the term “cycloalkoxy” refers to a group —OR′, where R′ is cycloalkyl as defined.

As used herein the term “alkoxycarbonyl” refers to groups such as:

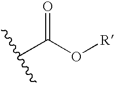

where the R′ represents an alkyl group as herein defined.

As used herein the term “aryloxycarbonyl” refers to groups such as:

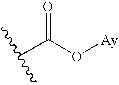

where the Ay represents an aryl group as herein defined.

As used herein the term “nitro” refers to a group —NO₂.

As used herein the term “cyano” refers to a group —CN.

As used herein the term “azido” refers to a group —N₃.

As used herein the term amino refers to a group —NR′R″, where R′ and R″ independently represent H, alkyl, alkenyl, alkynyl, cycloalkyl, heterocyclyl, aryl, or heteroaryl. Similarly, the term “alkylamino” includes an alkylene linker through which the amino group is attached.

As used herein the term “amide” refers to a group —C(O)NR′R″, where R′ and R″ independently represent H, alkyl, alkenyl, alkynyl, cycloalkyl, heterocyclyl, aryl, or heteroaryl.

As used herein throughout the present specification, the phrase “optionally substituted” or variations thereof denote an optional substitution, including multiple degrees of substitution, with one or more substituent group. The phrase should not be interpreted so as to be imprecise or duplicative of substitution patterns herein described or depicted specifically. Rather, those of ordinary skill in the art will appreciate that the phrase is included to provide for modifications, which are encompassed within the scope of the appended claims.

The compounds of the present invention may crystallize in more than one form, a characteristic known as polymorphism, and such polymorphic forms (“polymorphs”) are within the scope of the present invention. Polymorphism generally can occur as a response to changes in temperature, pressure, or both. Polymorphism can also result from variations in the crystallization process. Polymorphs can be distinguished by various physical characteristics known in the art such as x-ray diffraction patterns, solubility, and melting point. Though a crystalline form of compounds of the present invention are generally preferred, the invention also contemplates amorphous forms of the compounds produced by methods known in the art (e.g. spray drying, milling, freeze drying, and so forth).

Certain of the compounds described herein contain one or more chiral centers, or may otherwise be capable of existing as multiple stereoisomers. The scope of the present invention includes mixtures of stereoisomers as well as purified enantiomers or enantiomerically and/or diastereomerically enriched mixtures. Also included within the scope of the invention are the individual isomers of the compounds of the present invention, as well as any wholly or partially equilibrated mixtures thereof. The present invention also includes the individual isomers of the compounds represented by the formulas above as mixtures with isomers thereof in which one or more chiral centers are inverted.

As used herein, the term “solvate” refers to a complex of variable stoichiometry formed by a solute (in this invention, a compound of the present invention, or a salt or other pharmaceutically acceptable derivative thereof) and a solvent. Such solvents, for the purpose of the invention, should not interfere with the biological activity of the solute. Non-limiting examples of suitable solvents include, but are not limited to water, methanol, ethanol, ethyl acetate, acetone, acetonitrile, and acetic acid. Preferably the solvent used is a pharmaceutically acceptable solvent. Non-limiting examples of suitable pharmaceutically acceptable solvents include water, ethanol, and acetic acid. Most preferably the solvent used is water.

As used herein, the term “pharmaceutically acceptable derivative” means any pharmaceutically acceptable salt, ester, salt of an ester, ether, amides, or other derivative of a compound of this invention which, upon administration to a recipient, is capable of providing directly or indirectly a compound of this invention or an inhibitorily active metabolite or residue thereof. Particularly favored derivatives and prodrugs are those that increase the bioavailability of the compounds of this invention when such compounds are administered to a mammal, for example, by allowing an orally administered compound to be more readily absorbed into the blood, or which enhance delivery of the parent compound to a biological compartment, for example, the brain or lymphatic system, relative to the parent species.

Salts of the compounds of the present invention may be made by methods known to a person skilled in the art. For example, treatment of a compound of the present invention with an appropriate base or acid in an appropriate solvent will yield the corresponding salt.

Typically, but not absolutely, the salts of the present invention are pharmaceutically acceptable salts. Salts encompassed within the term “pharmaceutically acceptable salts” refer to non-toxic salts of the compounds of this invention. Salts of the compounds of the present invention may comprise acid addition salts. Representative salts include acetate, benzenesulfonate, benzoate, bicarbonate, bisulfate, bitartrate, borate, calcium edetate, camsylate, carbonate, clavulanate, citrate, dihydrochloride, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isethionate, lactate, lactobionate, laurate, malate, maleate, mandelate, mesylate, methylsulfate, monopotassium maleate, mucate, napsylate, nitrate, N-methylglucamine, oxalate, pamoate (embonate), palmitate, pantothenate, phosphate/diphosphate, polygalacturonate, potassium, salicylate, sodium, stearate, subacetate, succinate, sulfate, tannate, tartrate, teoclate, tosylate, triethiodide, trimethylammonium, and valerate salts. Other salts, which are not pharmaceutically acceptable, may be useful in the preparation of compounds of this invention and these should be considered to form a further aspect of the invention.

Pharmaceutically acceptable salts of the compounds according to the invention include those derived from pharmaceutically acceptable inorganic and organic acids and bases. Examples of suitable acids include hydrochloric, hydrobromic, sulfuric, nitric, perchloric, fumaric, maleic, phosphoric, glycollic, lactic, salicyclic, succinic, toluene-p-sulfonic, tartaric, acetic, citric, methanesulfonic, ethanesulfonic, formic, benzoic, malonic, naphthalene-2-sulfonic and benzenesulfonic acids. Other acids, such as oxalic, while not in themselves pharmaceutically acceptable, may be employed in the preparation of salts useful as intermediates in obtaining the compounds of the invention and their pharmaceutically acceptable acid addition salts.

Other compounds of this invention may be prepared by one skilled in the art following the teachings of the specification coupled with knowledge in the art using reagents that are readily synthesized or commercially available.

Any reference to any of the above compounds also includes a reference to a pharmaceutically acceptable salt thereof.

Esters of the compounds of the present invention are independently selected from the following groups: (1) carboxylic acid esters obtained by esterification of the hydroxy groups, in which the non-carbonyl moiety of the carboxylic acid portion of the ester grouping is selected from straight or branched chain alkyl (for example, acetyl, n-propyl, t-butyl, or n-butyl), alkoxyalkyl (for example, methoxymethyl), aralkyl (for example, benzyl), aryloxyalkyl (for example, phenoxymethyl), aryl (for example, phenyl optionally substituted by, for example, halogen, C₁₋₄alkyl, or C₁₋₄alkoxy or amino); (2) sulfonate esters, such as alkyl- or aralkylsulfonyl (for example, methanesulfonyl); (3) amino acid esters (for example, L-valyl or L-isoleucyl); (4) phosphonate esters and (5) mono-, di- or triphosphate esters. The phosphate esters may be further esterified by, for example, a C₁₋₂₀ alcohol or reactive derivative thereof, or by a 2,3-di (C₆₋₂₄)acyl glycerol.

In such esters, unless otherwise specified, any alkyl moiety present advantageously contains from 1 to 18 carbon atoms, particularly from 1 to 6 carbon atoms, more particularly from 1 to 4 carbon atoms, Any cycloalkyl moiety present in such esters advantageously contains from 3 to 6 carbon atoms. Any aryl moiety present in such esters advantageously comprises a phenyl group.

Ethers of the compounds of the present invention include, but are not limited to methyl, ethyl, butyl and the like.

As used herein, the term “effective amount” means that amount of a drug or pharmaceutical agent that will elicit the biological or medical response of a tissue, system, animal, or human that is being sought, for instance, by a researcher or clinician. The term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function.

The term “modulators” as used herein is intended to encompass antagonist, agonist, inverse agonist, partial agonist or partial antagonist, inhibitors and activators.

In one aspect of the present invention, the compounds demonstrate protective effects against HIV infection by inhibiting binding of HIV to a chemokine receptor such as CXCR4 of a target cell. The invention includes a method that comprises contacting the target cell with an amount of the compound that is effective at inhibiting the binding of the virus to the chemokine receptor.

In addition to the role chemokine receptors play in HIV infection this receptor class has also been implicated in a wide variety of diseases. Thus CXCR4 modulators may also have a therapeutic role in the treatment of diseases associated with hematopoiesis, including but not limited to, controlling the side effects of chemotherapy, enhancing the success of bone marrow transplantation, enhancing wound healing and burn treatment, as well as combating bacterial infections in leukemia. In addition, compounds may also have a therapeutic role in diseases associated with inflammation, including but not limited to inflammatory or allergic diseases such as asthma, allergic rhinitis, hypersensitivity lung diseases, hypersensitivity pneumonitis, eosinophilic pneumonitis, delayed-type hypersensitivity, interstitial lung disease (ILD) (e.g. idiopathic pulmonary fibrosis, or ILD associated with rheumatoid arthritis, systemic lupus erythematosus, ankylosing spondylitis, systemic sclerosis, Sjogren's syndrome, polymyositis or dermatomyositis), systemic anaphylaxis or hypersensitivity responses, drug allergies, insect sting allergies, autoimmune diseases such as rheumatoid arthritis, psoriatic arthritis, systemic lupus erythematosus, myastenia gravis, juvenile onset diabetes, glomerulonephritis, autoimmune throiditis, graft rejection, including allograft rejection or graft-versus-host disease, inflammatory bowel diseases, such as Crohn's disease and ulcerative colitus, spondyloarthropathies, scleroderma, psoriasis (including T-cell-mediated psoriasis) and inflammatory dermatoses such as dermatitis, eczema, atopic dermatitis, allergic contact dermatitis, urticaria, vasculitis (e.g. necrotizing, cutaneous, and hypersensitivity vasculitis), eoosinophilic myotis, eosinophilic fasciitis, and cancers.

The present invention features compounds according to the invention for use in medical therapy, for example for the treatment (including prophylaxis) of a viral infection, for example an HIV infection and associated conditions. The compounds according to the invention are especially useful for the treatment of AIDS and related clinical conditions such as AIDS related complex (ARC), progressive generalized lymphadenopathy (PGL), Kaposi's sarcoma, thromobocytopenic purpura, AIDS-related neurological conditions such as AIDS dementia complex, multiple sclerosis or tropical paraperesis, anti-HIV antibody-positive and HIV-positive conditions, including such conditions in asymptomatic patients.

The present invention further provides a method for the treatment of a clinical condition in a patient, for example, a mammal including a human which clinical condition includes those which have been discussed hereinbefore, which comprises treating said patient with a pharmaceutically effective amount of a compound according to the invention. The present invention also includes a method for the treatment (including prophylaxis) of any of the aforementioned diseases or conditions.

According to another aspect, the present invention provides a method for the treatment or prevention of the symptoms or effects of a viral infection in an infected patient, for example, a mammal including a human, which comprises administering to said patient a pharmaceutically effective amount of a compound according to the invention. According to one aspect of the invention, the viral infection is a retroviral infection, in particular an HIV infection.

The present invention further includes the use of a compound according to the invention in the manufacture of a medicament for administration to a subject for the treatment of a viral infection, in particular and HIV infection.

The compounds according to the invention may also be used in adjuvant therapy in the treatment of HIV infections or HIV-associated symptoms or effects, for example Kaposi's sarcoma. Reference herein to treatment extends to prophylaxis as well as the treatment of established conditions, disorders and infections, symptoms thereof, and associated clinical conditions. The above compounds according to the invention and their pharmaceutically acceptable derivatives may be employed in combination with other therapeutic agents for the treatment of the above infections or conditions. Combination therapies according to the present invention comprise the administration of a compound of the present invention or a pharmaceutically acceptable derivative thereof and another pharmaceutically active agent. The active ingredient(s) and pharmaceutically active agents may be administered simultaneously (i.e., concurrently) in either the same or different pharmaceutical compositions or sequentially in any order. The amounts of the active ingredient(s) and pharmaceutically active agent(s) and the relative timings of administration will be selected in order to achieve the desired combined therapeutic effect.

For use in therapy, therapeutically effective amounts of a compound of the present invention, as well as salts, solvates, or other pharmaceutically acceptable derivatives thereof, may be administered as the raw chemical. Additionally, the active ingredient may be presented as a pharmaceutical composition.

Accordingly, the invention further provides pharmaceutical compositions that include effective amounts of compounds of the present invention and salts, solvates, or other pharmaceutically acceptable derivatives thereof, and one or more pharmaceutically acceptable carriers, diluents, or excipients. The compounds of the present invention and salts, solvates, or other pharmaceutically acceptable derivatives thereof, are as herein described. The carrier(s), diluent(s) or excipient(s) must be acceptable, in the sense of being compatible with the other ingredients of the formulation and not deleterious to the recipient of the pharmaceutical composition.

In accordance with another aspect of the invention there is also provided a process for the preparation of a pharmaceutical formulation including admixing a compound of the present invention or salts, solvates, or other pharmaceutically acceptable derivatives thereof, with one or more pharmaceutically acceptable carriers, diluents or excipients.

A therapeutically effective amount of a compound of the present invention will depend upon a number of factors. For example, the species, age, and weight of the recipient, the precise condition requiring treatment and its severity, the nature of the formulation, and the route of administration are all factors to be considered. The therapeutically effective amount ultimately should be at the discretion of the attendant physician or veterinarian. Regardless, an effective amount of a compound of the present invention for the treatment of humans suffering from frailty, generally, should be in the range of 0.1 to 100 mg/kg body weight of recipient (mammal) per day. More usually the effective amount should be in the range of 0.1 to 10 mg/kg body weight per day. Thus, for a 70 kg adult mammal one example of an actual amount per day would usually be from 7 to 700 mg. This amount may be given in a single dose per day or in a number (such as two, three, four, five, or more) of sub-doses per day such that the total daily dose is the same. An effective amount of a salt, solvate, or other pharmaceutically acceptable derivative thereof, may be determined as a proportion of the effective amount of the compound of the present invention per se. Similar dosages should be appropriate for treatment of the other conditions referred to herein.

Pharmaceutical formulations may be presented in unit dose forms containing a predetermined amount of active ingredient per unit dose. Such a unit may contain, as a non-limiting example, 0.5 mg to 1 g of a compound of the formula (I), depending on the condition being treated, the route of administration, and the age, weight, and condition of the patient. Preferred unit dosage formulations are those containing a daily dose or sub-dose, as herein above recited, or an appropriate fraction thereof, of an active ingredient. Such pharmaceutical formulations may be prepared by any of the methods well known in the pharmacy art.

Pharmaceutical formulations may be adapted for administration by any appropriate route, for example by an oral (including buccal or sublingual), rectal, nasal, topical (including buccal, sublingual or transdermal), vaginal, or parenteral (including subcutaneous, intramuscular, intravenous or intradermal) route. Such formulations may be prepared by any method known in the art of pharmacy, for example by bringing into association the active ingredient with the carrier(s) or excipient(s). By way of example, and not meant to limit the invention, with regard to certain conditions and disorders for which the compounds of the present invention are believed useful certain routes will be preferable to others.

Pharmaceutical formulations adapted for oral administration may be presented as discrete units such as capsules or tablets; powders or granules; solutions or suspensions, each with aqueous or non-aqueous liquids; edible foams or whips; or oil-in-water liquid emulsions or water-in-oil liquid emulsions. For instance, for oral administration in the form of a tablet or capsule, the active drug component can be combined with an oral, non-toxic pharmaceutically acceptable inert carrier such as ethanol, glycerol, water, and the like. Generally, powders are prepared by comminuting the compound to a suitable fine size and mixing with an appropriate pharmaceutical carrier such as an edible carbohydrate, as, for example, starch or mannitol. Flavorings, preservatives, dispersing agents, and coloring agents can also be present.

Capsules are made by preparing a powder, liquid, or suspension mixture and encapsulating with gelatin or some other appropriate shell material. Glidants and lubricants such as colloidal silica, talc, magnesium stearate, calcium stearate, or solid polyethylene glycol can be added to the mixture before the encapsulation. A disintegrating or solubilizing agent such as agar-agar, calcium carbonate or sodium carbonate can also be added to improve the availability of the medicament when the capsule is ingested. Moreover, when desired or necessary, suitable binders, lubricants, disintegrating agents, and coloring agents can also be incorporated into the mixture. Examples of suitable binders include starch, gelatin, natural sugars such as glucose or beta-lactose, corn sweeteners, natural and synthetic gums such as acacia, tragacanth, or sodium alginate, carboxymethylcellulose, polyethylene glycol, waxes, and the like. Lubricants useful in these dosage forms include, for example, sodium oleate, sodium stearate, magnesium stearate, sodium benzoate, sodium acetate, sodium chloride, and the like. Disintegrators include, without limitation, starch, methyl cellulose, agar, bentonite, xanthan gum, and the like.

Tablets are formulated, for example, by preparing a powder mixture, granulating or slugging, adding a lubricant and disintegrant, and pressing into tablets. A powder mixture may be prepared by mixing the compound, suitably comminuted, with a diluent or base as described above. Optional ingredients include binders such as carboxymethylcellulose, aliginates, gelatins, or polyvinyl pyrrolidone, solution retardants such as paraffin, resorption accelerators such as a quaternary salt, and/or absorption agents such as bentonite, kaolin, or dicalcium phosphate. The powder mixture can be wet-granulated with a binder such as syrup, starch paste, acadia mucilage or solutions of cellulosic or polymeric materials, and forcing through a screen. As an alternative to granulating, the powder mixture can be run through the tablet machine and the result is imperfectly formed slugs broken into granules. The granules can be lubricated to prevent sticking to the tablet-forming dies by means of the addition of stearic acid, a stearate salt, talc or mineral oil. The lubricated mixture is then compressed into tablets. The compounds of the present invention can also be combined with a free flowing inert carrier and compressed into tablets directly without going through the granulating or slugging steps. A clear or opaque protective coating consisting of a sealing coat of shellac, a coating of sugar or polymeric material, and a polish coating of wax can be provided. Dyestuffs can be added to these coatings to distinguish different unit dosages.

Oral fluids such as solutions, syrups, and elixirs can be prepared in dosage unit form so that a given quantity contains a predetermined amount of the compound. Syrups can be prepared, for example, by dissolving the compound in a suitably flavored aqueous solution, while elixirs are prepared through the use of a non-toxic alcoholic vehicle. Suspensions can be formulated generally by dispersing the compound in a non-toxic vehicle. Solubilizers and emulsifiers such as ethoxylated isostearyl alcohols and polyoxy ethylene sorbitol ethers, preservatives; flavor additives such as peppermint oil, or natural sweeteners, saccharin, or other artificial sweeteners; and the like can also be added.

Where appropriate, dosage unit formulations for oral administration can be microencapsulated. The formulation can also be prepared to prolong or sustain the release as for example by coating or embedding particulate material in polymers, wax or the like.

The compounds of the present invention and salts, solvates, or other pharmaceutically acceptable derivatives thereof, can also be administered in the form of liposome delivery systems, such as small unilamellar vesicles, large unilamellar vesicles, and multilamellar vesicles. Liposomes can be formed from a variety of phospholipids, such as cholesterol, stearylamine, or phosphatidylcholines.

The compounds of the present invention and salts, solvates, or other pharmaceutically acceptable derivatives thereof may also be delivered by the use of monoclonal antibodies as individual carriers to which the compound molecules are coupled.

The compounds may also be coupled with soluble polymers as targetable drug carriers. Such polymers can include polyvinylpyrrolidone (PVP), pyran copolymer, polyhydroxypropylmethacrylamide-phenol, polyhydroxyethyl-aspartamidephenol, or polyethyleneoxidepolylysine substituted with palmitoyl residues. Furthermore, the compounds may be coupled to a class of biodegradable polymers useful in achieving controlled release of a drug; for example, polylactic acid, polyepsilon caprolactone, polyhydroxy butyric acid, polyorthoesters, polyacetals, polydihydropyrans, polycyanoacrylates, and cross-linked or amphipathic block copolymers of hydrogels.

Pharmaceutical formulations adapted for transdermal administration may be presented as discrete patches intended to remain in intimate contact with the epidermis of the recipient for a prolonged period of time. For example, the active ingredient may be delivered from the patch by iontophoresis as generally described in Pharmaceutical Research, 3(6), 318 (1986), incorporated herein by reference as related to such delivery systems.

Pharmaceutical formulations adapted for topical administration may be formulated as ointments, creams, suspensions, lotions, powders, solutions, pastes, gels, sprays, aerosols, or oils.

For treatments of the eye or other external tissues, for example mouth and skin, the formulations may be applied as a topical ointment or cream. When formulated in an ointment, the active ingredient may be employed with either a paraffinic or a water-miscible ointment base. Alternatively, the active ingredient may be formulated in a cream with an oil-in-water cream base or a water-in-oil base.

Pharmaceutical formulations adapted for topical administrations to the eye include eye drops wherein the active ingredient is dissolved or suspended in a suitable carrier, especially an aqueous solvent.

Pharmaceutical formulations adapted for topical administration in the mouth include lozenges, pastilles, and mouthwashes.

Pharmaceutical formulations adapted for nasal administration, where the carrier is a solid, include a coarse powder having a particle size for example in the range 20 to 500 microns. The powder is administered in the manner in which snuff is taken, i.e., by rapid inhalation through the nasal passage from a container of the powder held close up to the nose. Suitable formulations wherein the carrier is a liquid, for administration as a nasal spray or as nasal drops, include aqueous or oil solutions of the active ingredient.

Pharmaceutical formulations adapted for administration by inhalation include fine particle dusts or mists, which may be generated by means of various types of metered dose pressurized aerosols, nebulizers, or insufflators.

Pharmaceutical formulations adapted for rectal administration may be presented as suppositories or as enemas.

Pharmaceutical formulations adapted for vaginal administration may be presented as pessaries, tampons, creams, gels, pastes, foams, or spray formulations.

Pharmaceutical formulations adapted for parenteral administration include aqueous and non-aqueous sterile injection solutions which may contain anti-oxidants, buffers, bacteriostats, and solutes that render the formulation isotonic with the blood of the intended recipient; and aqueous and non-aqueous sterile suspensions which may include suspending agents and thickening agents. The formulations may be presented in unit-dose or multi-dose containers, for example sealed ampules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules, and tablets.

In addition to the ingredients particularly mentioned above, the formulations may include other agents conventional in the art having regard to the type of formulation in question. For example, formulations suitable for oral administration may include flavoring or coloring agents.

The compounds of the present invention and their salts, solvates, or other pharmaceutically acceptable derivatives thereof, may be employed alone or in combination with other therapeutic agents. The compound(s) of the present invention and the other pharmaceutically active agent(s) may be administered together or separately and, when administered separately, administration may occur simultaneously or sequentially, in any order. The amounts of the compound(s) of the present invention and the other pharmaceutically active agent(s) and the relative timings of administration will be selected in order to achieve the desired combined therapeutic effect. The administration in combination of a compound of the present invention and salts, solvates, or other pharmaceutically acceptable derivatives thereof with other treatment agents may be in combination by administration concomitantly in: (1) a unitary pharmaceutical composition including both compounds; or (2) separate pharmaceutical compositions each including one of the compounds. Alternatively, the combination may be administered separately in a sequential manner wherein one treatment agent is administered first and the other second or vice versa. Such sequential administration may be close in time or remote in time.

The present invention may be used in combination with one or more agents useful in the prevention or treatment of HIV. Examples of such agents include:

Nucleotide reverse transcriptase inhibitors such as zidovudine, didanosine, lamivudine, zalcitabine, abacavir, stavidine, adefovir, adefovir dipivoxil, fozivudine, todoxil, emtricitabine, alovudine, amdoxovir, elvucitabine, and similar agents;

Non-nucleotide reverse transcriptase inhibitors (including an agent having anti-oxidation activity such as immunocal, oltipraz, etc.) such as nevirapine, delavirdine, efavirenz, loviride, immunocal, oltipraz, capravirine, TMC-278, TMC-125, etravirine, and similar agents;

Protease inhibitors such as saquinavir, ritonavir, indinavir, nelfinavir, amprenavir, fosamprenavir, brecanavir, atazanavir, tipranavir, palinavir, lasinavir, and similar agents;

Entry inhibitors such as enfuvirtide (T-20), T-1249, PRO-542, PRO-140, TNX-355, BMS-806, 5-Helix and similar agents;

Integrase inhibitors such as L-870,180 and similar agents;

Budding inhibitors such as PA-344 and PA-457, and similar agents; and

Other CXCR4 and/or CCR5 inhibitors such as vicriviroc (Sch-C), Sch-D, TAK779, maraviroc (UK 427,857), TAK449, as well as those disclosed in WO 02/74769, PCT/US03/39644, PCT/US03/39975, PCT/US03/39619, PCT/US03/39618, PCT/US03139740, and PCT/US03/39732, and similar agents.

The scope of combinations of compounds of this invention with HIV agents is not limited to those mentioned above, but includes in principle any combination with any pharmaceutical composition useful for the treatment of HIV. As noted, in such combinations the compounds of the present invention and other HIV agents may be administered separately or in conjunction. In addition, one agent may be prior to, concurrent to, or subsequent to the administration of other agent(s).

The compounds of the present invention may be used in the treatment of a variety of disorders and conditions and, as such, the compounds of the present invention may be used in combination with a variety of other suitable therapeutic agents useful in the treatment (including prophylaxis) of those disorders or conditions. The compounds may be used in combination with any other pharmaceutical composition where such combined therapy may be useful to modulate chemokine receptor activity and thereby prevent and treat inflammatory and/or immunoregulatory diseases.

It should be understood that in addition to the ingredients particularly mentioned above, the pharmaceutical compositions of this invention may include other agents conventional in the art having regard to the type of pharmaceutical composition in question, for example, those suitable for oral administration may include such further agents as sweeteners, thickeners, and flavoring agents.

The compounds of the present invention may be prepared according to the following reaction schemes and examples, or modifications thereof using readily available starting materials, reagents and conventional synthesis procedures. In these reactions, it is also possible to make use of variants which are know to those of ordinary skill in the art.

In all of the examples described below, protecting groups for sensitive or reactive groups are employed where necessary in accordance with general principles of synthetic chemistry. Protecting groups are manipulated according to standard methods of organic synthesis (T. W. Green and P. G. M. Wuts (1991) Protecting Groups in Organic Synthesis, John Wiley & Sons, incorporated by reference with regard to protecting groups). These groups are removed at a convenient stage of the compound synthesis using methods that are readily apparent to those skilled in the art. The selection of processes as well as the reaction conditions and order of their execution shall be consistent with the preparation of compounds of the present invention.

Those skilled in the art will recognize if a stereocenter exists in compounds of the present invention. Accordingly, the scope of the present invention includes all possible stereoisomers and includes not only racemic compounds but the individual enantiomers as well. When a compound is desired as a single enantiomer, such may be obtained by stereospecific synthesis, by resolution of the final product or any convenient intermediate, or by chiral chromatographic methods as are known in the art. Resolution of the final product, an intermediate, or a starting material may be affected by any suitable method known in the art. See, for example, Stereochemistry of Organic Compounds by E. L. Eliel, S. H. Wilen, and L. N. Mander (Wiley-Interscience, 1994), incorporated by reference with regard to stereochemistry.

### EXPERIMENTAL SECTION

### Abbreviations

As used herein the symbols and conventions used in these processes, schemes and examples are consistent with those used in the contemporary scientific literature, for example, the Journal of the American Chemical Society or the Journal of Biological Chemistry. Specifically, the following abbreviations may be used in the examples and throughout the specification:
g (grams); mg (milligrams);
L (liters); mL (milliliters);
μL (microliters); psi (pounds per square inch);
M (molar); mM (millimolar);
Hz (Hertz); MHz (megahertz);
mol (moles); mmol (millimoles);
RT (room temperature); h (hours);
min (minutes); TLC (thin layer chromatography);
mp (melting point); RP (reverse phase);
Tᵣ (retention time); TFA (trifluoroacetic acid);
TEA (triethylamine); THF (tetrahydrofuran);
TFAA (trifluoroacetic anhydride); CD₃OD (deuterated methanol);
CDCl₃ (deuterated chloroform); DMSO (dimethylsulfoxide);
SiO₂ (silica); atm (atmosphere);
EtOAc (ethyl acetate); CHCl₃ (chloroform);
HCl (hydrochloric acid); Ac (acetyl);
DMF (N,N-dimethylformamide); Me (methyl);
Cs₂CO₃ (cesium carbonate); EtOH (ethanol);
Et (ethyl); tBu (tert-butyl);
MeOH (methanol) p-TsOH (p-toluenesulfonic acid);
MP-TsOH (polystyrene resin bound equivalent of p-TsOH from Argonaut Technologies).

Unless otherwise indicated, all temperatures are expressed in ° C. (degrees Centigrade). All reactions conducted at room temperature unless otherwise noted.

¹H-NMR spectra were recorded on a Varian VXR-300, a Varian Unity-300, a Varian Unity-400 instrument, or a General Electric QE-300. Chemical shifts are expressed in parts per million (ppm, δ units). Coupling constants are in units of hertz (Hz). Splitting patterns describe apparent multiplicities and are designated as s (singlet), d (doublet), t (triplet), q (quartet), m (multiplet), or br (broad).

Mass spectra were obtained on Micromass Platform or ZMD mass spectrometers from Micromass Ltd., Altricham, UK, using either Atmospheric Chemical Ionization (APCI) or Electrospray Ionization (ESI).

Analytical thin layer chromatography was used to verify the purity of intermediate(s) which could not be isolated or which were too unstable for full characterization as well as to follow the progress of reaction(s).

The absolute configuration of compounds was assigned by Ab Initio Vibrational Circular Dichroism (VCD) Spectroscopy. The experimental VCD spectra were acquired in CDCl₃ using a Bomem Chiral® VCD spectrometer operating between 2000 and 800 cm⁻¹. The Gaussian 98 Suite of computational programs was used to calculate model VCD spectrums. The stereochemical assignments were made by comparing this experimental spectrum to the VCD spectrum calculated for a model structure with (R)- or (S)-configuration. Incorporated by reference with regard to such spectroscopy are: J. R. Chesseman, M. J. Frisch, F. J. Devlin and P. J. Stephens, Chem. Phys. Lett. 252 (1996) 211; P. J. Stephens and F. J. Devlin, Chirality 12 (2000) 172; and Gaussian 98, Revision A.11.4, M. J. Frisch et al., Gaussian, Inc., Pittsburgh Pa., 2002.

Compounds of formula (I) can be prepared according to Scheme 1.

Compounds of formula (I) where R is H, t is 1 and all other variables are as defined herein can be prepared according to Scheme 1:

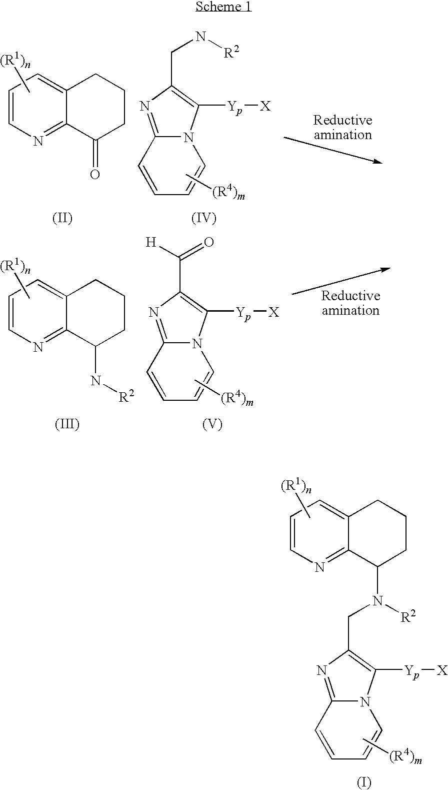

More specifically, compounds of formula (I) can be prepared by reacting a compound of formula (II) with a compound (IV) or alternatively reacting a compound of formula (III) with a compound of formula (V) under reductive conditions. The reductive amination can be carried out by treating the compound of formula (II) or (III) with a compound of formula (IV) or (V) in an inert solvent in the presence of a reducing agent. The reaction may be heated to 50-150° C. or performed at ambient temperature. Suitable solvents include dichloromethane, dichloroethane, tetrahydrofuran, acetonitrile, toluene, and the like. The reducing agent is typically sodium borohydride, sodium cyanoborohydride, sodium triacetoxyborohydride, and the like. Optionally the reaction can be run in presence of acid, such as acetic acid and the like.

Compounds of formula (II) can be prepared as described in the literature (J. Org. Chem., 2002, 67, 2197-2205, herein incorporated by reference with regard to such synthesis). Compounds of formula (III) can be prepared by reductive amination of compounds of formula (II) using processes well known to those skilled in the art of organic synthesis. Compounds of formula (V) can be prepared by methods similar to those described in the literature (J. Heterocyclic Chemistry, 1992, 29, 691-697; J. Org. Chem., 2000, 65, 6572-6575; Chem. Pharm. Bull. 2000, 48, 935-940; incorporated by reference with regard to such synthesis). Compounds of formula (IV) can be prepared from compounds of formula (V) via reductive amination using processes known to those skilled in the art.

As is evident to one skilled in the art a compound where R is not H can be prepared in a similar fashion as outlined in Scheme 1. Also evident to one skilled in the art is that compounds of formula I where t is 0 or t is 2 can be prepared in a similar fashion as outlined in Scheme 1.

Compounds of formula (I) wherein R is H, t is 1, LV is a suitable leaving group (e.g., halogen, mesylate, tosylate, or the like) and all other variables are as defined in connection with formula (I) can be prepared according to Scheme 2:

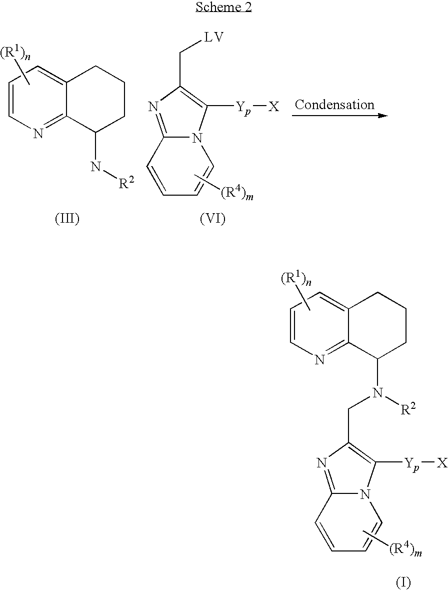

Compound of formula (I) can be prepared by reacting a compound of formula (III) with a compound of formula (VI) where LV is a leaving group (e.g., halogen, mesylate, tosylate, or the like). This condensation is typically carried out in a suitable solvent optionally in the presence of base, optionally with heating. Suitable solvents include tetrahydrofuran, dioxane, acetonitrile, nitromethane, N,N-dimethylformamide, and the like. Suitable bases include triethylamine, pyridine, dimethylaminopyridine, N,N-diisopropylethylamine, potassium carbonate, sodium carbonate, cesium carbonate and the like. The reaction can be carried out at room temperature or optionally heated to 30-200° C. Optionally the reaction can be carried out in a microwave. A catalyst, such as potassium iodide, tertbutylammonium iodide, or the like, can optionally be added to the reaction mixture. Compounds of formula (VI) can be prepared by methods similar to those described in the literature (Chem. Pharm. Bull. 2000, 48, 935; Tetrahedron, 1991, 47, 5173; Tetrahedron Lett. 1990, 31, 3013; J. Heterocyclic Chemistry, 1988, 25, 129; Chemistry of Heterocyclic Compounds, 2002, 38, 590; each incorporated by reference with regard to such synthesis).

### EXAMPLES

### Example 1

### (8S)-N-Methyl-N-{[3-(4-pyridinyl)imidazo[1,2-a]pyridin-2-yl]methyl}-5,6,7,8-tetrahydro-8-quinolinamine

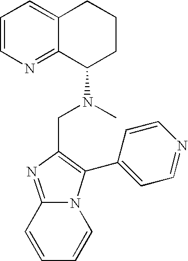

### a) Ethyl 3-iodoimidazo[1,2-a]pyridine-2-carboxylate

A solution of ethyl imidazo[1,2-a]pyridine-2-carboxylate (11.43 g, 60.1 mmol, J. Org. Chem., 1965, 30, 2403-2407) in glacial acetic acid (100 mL) was treated with N-iodosuccinimide (14.87 g, 66.1 mmol). After stirring at rt for 30 min, the reaction mixture was concentrated to dryness. The residue was taken up in dichloromethane and washed with a 1:1:1 mixture of 10% aqueous Na₂CO₃, brine and saturated Na₂S₂O₃. The aqueous layer was extracted with additional dichloromethane (2×). The combined organic layers were dried over Na₂SO₄ and concentrated. Flash chromatography (silica gel, 0 to 100% ethyl acetate in hexanes) afforded ethyl 3-iodoimidazo[1,2-a]pyridine-2-carboxylate as a pale yellow solid in quantitative yield. ¹H NMR (DMSO-d₆): δ 8.41 (d, 1H), 7.62 (m, 1H), 7.41 (m, 1H), 7.11 (m, 1H), 4.30 (q, 2H), 1.31 (t, 3H). MS m/z 317 (M+1).

### b) Ethyl 3-(4-pyridinyl)imidazo[1,2-a]pyridine-2-carboxylate

A solution of ethyl 3-iodoimidazo[1,2-a]pyridine-2-carboxylate (362 mg, 1.15 mmol) in 1,2-dimethoxyethane (8 mL) was treated with Pd(PPh₃)₄ (66 mg, 0.05 mmol), 4-pyridineboronic acid (155 mg, 1.26 mmol) and a solution of Na₂CO₃ (243 mg, 2.29 mmol) in 4 mL water. The reaction was heated in a 75° bath for 16 h, then another 155 mg 4-pyridineboronic acid was added. After 2 h, 53 mg Pd(PPh₃)₄ was added. When the reaction was judged to be mostly complete by LC/MS, it was cooled to rt and diluted with dichloromethane, then poured into water. The layers were separated and the aqueous layer was extracted with additional dichloromethane (5×). The combined organic layers were washed with brine, dried over Na₂SO₄ and concentrated. Flash chromatography (silica gel, 0 to 4% methanol in dichloromethane) afforded 92 mg (30%) of ethyl 3-(4-pyridinyl)imidazo[1,2-a]pyridine-2-carboxylate as a pale yellow solid. ¹H NMR (DMSO-d₆): δ 8.74 (m, 2H), 8.18 (d, 1H), 7.72 (m, 1H), 7.61 (m, 2H), 7.43 (m, 1H), 7.00 (t, 1H), 4.17 (q, 2H), 1.14 (t, 3H). MS m/z 268 (M+1).

### c) [3-(4-Pyridinyl)imidazo[1,2-a]pyridin-2-yl]methanol

A solution of ethyl 3-(4-pyridinyl)imidazo[1,2-a]pyridine-2-carboxylate (91 mg, 0.34 mmol) in tetrahydrofuran (5 mL) and methanol (2 mL) was cooled to 0°. A solution of lithium borohydride (0.85 mL, 2.0 M in THF) was added dropwise, then the reaction was allowed to warm to it and stirred overnight. The reaction was treated with 1N sodium hydroxide (3 mL) and stirred 30 min, then poured into water and extracted with ethyl acetate (5×). The combined organic layers were washed with brine, dried over Na₂SO₄ and concentrated. The aqueous layer was concentrated and the residue was slurried in brine and extracted with ethyl acetate (5×). The combined organic layers were washed with brine, dried over Na₂SO₄ and concentrated. The solids from both extractions were combined and purified by flash chromatography (silica gel, 0 to 10% methanol in dichloromethane) to afford 41 mg (53%) of [3-(4-pyridinyl)imidazo[1,2-a]pyridin-2-yl]methanol as a white solid. ¹H NMR (CD₃OD): δ 8.72 (m, 2H), 8.49 (d, 1H), 7.75 (m, 2H), 7.64 (m, 1H), 7.44 (m, 1H), 7.03 (t, 1H), 4.73 (s, 2H).

### d) 3-(4-Pyridinyl)imidazo[1,2-a]pyridine-2-carbaldehyde

A solution of [3-(4-pyridinyl)imidazo[1,2-a]pyridin-2-yl]methanol (39 mg, 0.17 mmol) in 1:1 acetonitrile/chloroform (10 mL) was treated with manganese dioxide (150 mg, 1.73 mmol) and stirred overnight. The reaction mixture was filtered through Celite and concentrated to afford 34 mg (87%) of 3-(4-pyridinyl)imidazo[1,2-a]pyridine-2-carbaldehyde as an off-white solid. ¹H NMR (CD₃OD): δ 10.08 (s, 1H), 8.79 (m, 2H), 8.41 (m, 1H), 7.78-7.72 (m, 3H), 7.55 (m, 1H), 7.11 (m, 1H). MS m/z 224 (M+1).

### e) (8S)-N-{(1S)-1-[4-(Methyloxy)phenyl]ethyl}-5,6,7,8-tetrahydro-8-quinolinamine

A solution of (S)-(−)-1-(4-methoxyphenyl)ethylamine (25.0 g, 166 mmol) and 6,7-dihydro-8(5H)-quinolinone (24.0 g, 166 mmol, J. Org. Chem., 2002, 67, 2197-2205) in dichloromethane was treated with glacial acetic acid (14.0 mL, 249 mmol) and sodium triacetoxyborohydride (53.0 g, 249 mmol). The reaction mixture was stirred at room temperature for 15 hours and then treated with sodium carbonate (106 g, 996 mmol) dissolved in water. The resulting mixture was stirred for 30 minutes and then diluted with dichloromethane. The phases were separated and the aqueous solution extracted with an additional portion of dichloromethane. The combined organic solutions were dried over MgSO₄ and concentrated to dryness at reduced pressure. The crude product was purified by flash chromatography (silica gel, gradient elution of dichloromethane to 97:3 dichloromethane/2M ammonia in MeOH) followed by recrystallization from hexane to afford 33 g (70%) of (8S)-N-{(1S)-1-[4-(methyloxy)phenyl]ethyl}-5,6,7,8-tetrahydro-8-quinolinamine as a white crystalline solid. ¹H NMR (CDCl₃): δ 8.40 (m, 1H), 7.33 (m, 3H), 7.04 (m, 1H), 6.84 (d, 2H), 4.02 (m, 1H), 3.83-3.78 (m, 4H), 2.73-2.62 (m, 2H), 1.82 (m, 1H), 1.72 (m, 1H), 1.57 (m, 2H), 1.43 (d, 3H).

### f) (8S)-N-Methyl-N-{(1S)-1-[4-(methyloxy)phenyl]ethyl}-5,6,7,8-tetrahydro-8-quinolinamine

To a stirred mixture of (8S)-N-{(1S)-1-[4-(methyloxy)phenyl]ethyl}-5,6,7,8-tetrahydro-8-quinolinamine (5.00 g, 17.7 mmol), 37% aqueous formaldehyde (2.90 mL, 35.4 mmol), and glacial acetic acid (1.52 mL, 26.6 mmol) in 50 mL of 1,2-dichloroethane was added NaBH(OAc)₃ (5.64 g, 26.6 mmol). After stirring at RT for 2 hours the mixture was diluted with 50 mL of dichloromethane followed by 80 mL of 10% aqueous Na₂CO₃. The resulting mixture was stirred vigorously for 30 minutes and then the phases separated. The aqueous phase was extracted twice with dichloromethane. The combined organic solutions were washed with saturated aqueous brine, dried over Na₂SO₄, and concentrated to dryness at reduced pressure to afford (8S)-N-methyl-N-{(1S)-1-[4-(methyloxy)phenyl]ethyl}-5,6,7,8-tetrahydro-8-quinolinamine in quantitative yield as a yellow oil. ¹H NMR (CDCl₃): δ 8.47 (d, 1H), 7.39 (d, 2H), 7.30 (d, 1H), 6.99 (dd, 1H), 6.84 (d, 2H), 4.42 (q, 1H), 3.97 (t, 1H), 3.78 (s, 3H), 2.79 (m, 1H), 2.61 (m, 1H), 2.05-1.78 (m, 6H), 1.57 (m, 1H), 1.37 (d, 3H). MS m/z 297 (M+H).

### g) (8S)-N-Methyl-5,6,7,8-tetrahydro-8-quinolinamine

A solution of (8S)-N-methyl-N-{(1S)-1-[4-(methyloxy)phenyl]ethyl}-5,6,7,8-tetrahydro-8-quinolinamine (5.48 g, 18.5 mmol) in 70 mL of 1:1 trifluoroacetic acid/dichloromethane was stirred at RT for 2.5 hours and then concentrated to dryness by rotary evaporation.

The resulting purple syrup was partitioned between 0.5N aqueous HCl and EtOAc. The phases were separated, the aqueous solution washed with EtOAc (3×), and the EtOAc solutions discarded. The aqueous solution was treated with 5N aqueous NaOH to pH=12. The resulting oily mixture was extracted with dichloromethane (5×). The combined dichloromethane extracts were dried over Na₂SO₄ and concentrated to dryness at reduced pressure to afford 2.76 g (92%) of (8S)-N-methyl-5,6,7,8-tetrahydro-8-quinolinamine as a yellow oil. ¹H NMR (CDCl₃): δ 8.37 (d, 1H), 7.34 (d, 1H), 7.03 (dd, 1H), 3.63 (t, 1H), 2.86-2.60 (m, 3H), 2.52 (s, 3H), 2.10 (m, 1H), 1.96 (m, 1H), 1.82-1.64 (m, h) (8S)-N-Methyl-N-{[3-(4-pyridinyl)imidazo[1,2-a]pyridin-2-yl]methyl}-5,6,7,8-tetrahydro-8-quinolinamine.

A solution of 3-(4-pyridinyl)imidazo[1,2-a]pyridine-2-carbaldehyde (33 mg, 0.15 mmol) and (8S)-N-methyl-5,6,7,8-tetrahydro-8-quinolinamine (24 mg, 0.15 mmol) in 1,2-dichloroethane (5 mL) was treated with glacial acetic acid (13 μL, 0.22 mmol). To this was added NaBH(OAc)₃ in portions over 3 min. After stirring overnight, the reaction was treated with 10% aqueous Na₂CO₃ and stirred 15 min. The mixture was extracted with dichloromethane. The organic layer was dried over Na₂SO₄ and concentrated. Flash chromatography (silica gel, 0 to 10% NH₄OH in acetonitrile) afforded 49 mg (89%) of (8S)-N-methyl-N-{[3-(4-pyridinyl)imidazo[1,2-a]pyridin-2-yl]methyl}-5,6,7,8-tetrahydro-8-quinolinamine as a brown oil. ¹H NMR (CD₃OD): δ 8.61 (m, 2H), 8.42 (d, 1H), 8.22 (d, 1H), 7.75 (m, 2H), 7.61 (m, 1H), 7.47-7.38 (m, 2H), 7.12 (m, 1H), 6.98 (t, 1H), 3.96-3.84 (m, 3H), 2.86-2.59 (m, 2H), 2.29 (s, 3H), 2.12-1.94 (m, 3H), 1.66 (m, 1H). MS m/z 370 (M+1).

### Example 2

### (8S)-N-{[3-(3-Aminopropyl)imidazo[1,2-a]pyridin-2-yl]methyl}-N-methyl-5,6,7,8-tetrahydro-8-quinolinamine

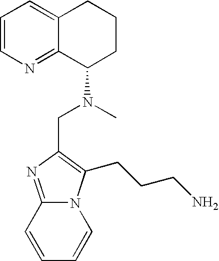

### a) Ethyl 3-[(E)-2-cyanoethenyl]imidazo[1,2-a]pyridine-2-carboxylate

A solution of ethyl 3-iodoimidazo[1,2-a]pyridine-2-carboxylate (0.50 g, 1.58 mmol) in DMF (15 mL) was degassed with nitrogen for 15 min. To this was added Pd(OAc)₂ (36 mg, 0.16 mmol), triphenylphosphine (124 mg, 0.47 mmol), acrylonitrile (0.60 mL, 15.8 mmol) and triethylamine (1.8 mL, 12.7 mmol). The reaction was heated in a 90° bath for 20 h, then cooled to rt. The mixture was poured into 10% aqueous Na₂CO₃ and extracted with ethyl acetate (3×). The combined organic layers were washed with brine, dried over Na₂SO₄ and concentrated. Flash chromatography (silica gel, 0 to 100% ethyl acetate in hexanes) afforded 295 mg (78%) of ethyl 3-[(E)-2-cyanoethenyl]imidazo[1,2-a]pyridine-2-carboxylate as a mixture of E and Z isomers. ¹H NMR (DMSO-d₆): δ 8.68 and 8.44 (d, 1H), 8.17 and 7.80 (d, 1H), 7.80 (m, 1H), 7.58 (m, 1H), 7.21 (m, 1H), 6.73 and 6.26 (d, 1H), 4.29 (m, 2H), 1.33 (m, 3H). MS m/z 242 (M+1).

### b) 1,1-Dimethylethyl{3-[2-(hydroxymethyl)imidazo[1,2-a]pyridin-3-yl]propyl}carbamate

A solution of ethyl 3-[(E)-2-cyanoethenyl]imidazo[1,2-a]pyridine-2-carboxylate (132 mg, 0.55 mmol) in 7N NH₃ in MeOH (approx. 60 mL) was hydrogenated over Raney nickel at 40 psi for 1.5 h. The reaction was filtered through Celite and concentrated. Purification by flash chromatography (silica gel, 0 to 10% NH₄OH in acetonitrile) afforded 59 mg of a mixture of ethyl 3-(3-aminopropyl)imidazo[1,2-a]pyridine-2-carboxylate and methyl 3-(3-aminopropyl)imidazo[1,2-a]pyridine-2-carboxylate as a yellow oil. This mixture was dissolved in dichloromethane (5 mL) and treated with di-t-butyl dicarbonate (80 mg, 0.37 mmol). After 18 h, the reaction was diluted with dichloromethane and washed with 10% aqueous citric acid (1×), 10% aqueous Na₂CO₃ (1×), brine (1×), dried over Na₂SO₄, and concentrated. Flash chromatography afforded 79 mg of a 2:1 mixture of ethyl 3-[3-({[(1,1-dimethylethyl)oxy]carbonyl}amino)propyl]imidazo[1,2-a]pyridine-2-carboxylate and methyl 3-[3-({[(1,1-dimethylethyl)oxy]carbonyl}amino)propyl]imidazo[1,2-a]pyridine-2-carboxylate as a colorless oil. MS m/z 348, 334 (M+1). The mixture was dissolved in tetrahydrofuran (5 mL), cooled to 0° and treated with lithium borohydride (0.63 mL, 2.0 M in THF). The reaction was allowed to warm to rt after 15 min, then stirred 4 h. After being treated with 1 N sodium hydroxide (5 mL) and stirring 5 min, the reaction was concentrated. The residue was partitioned between ethyl acetate and water. The aqueous layer was back-extracted with ethyl acetate (1×). The combined organic layers were washed with brine, dried over Na₂SO₄ and concentrated to a colorless oil. Flash chromatography (silica gel, 0 to 10% 2N NH₃/MeOH in dichloromethane) afforded 37 mg (22% over 3 steps) of 1,1-dimethylethyl {3-[2-(hydroxymethyl)imidazo[1,2-a]pyridin-3-yl]propyl}carbamate as a colorless oil. ¹H NMR (CD₃OD): δ 8.23 (d, 1H), 7.48 (m, 1H), 7.27 (m, 1H), 6.93 (m, 1H), 4.72 (s, 2H), 3.05 (m, 4H), 1.82 (m, 2H), 1.41 (s, 9H). MS m/z 306 (M+1).

### c) 1,1-Dimethylethyl[3-(2-formylimidazo[1,2-a]pyridin-3-yl)propyl]carbamate

Reaction of 1,1-dimethylethyl {3-[2-(hydroxymethyl)imidazo[1,2-a]pyridin-3-yl]propyl}carbamate (163 mg, 0.53 mmol) as described herein for the preparation of 3-(4-pyridinyl)imidazo[1,2-a]pyridine-2-carbaldehyde afforded 138 mg (85%) of 1,1-dimethylethyl[3-(2-formylimidazo[1,2-a]pyridin-3-yl)propyl]carbamate as a colorless oil. ¹H NMR (CD₃OD): δ 10.13 (s, 1H), 8.37 (m, 1H), 7.60 (m, 1H), 7.41 (m, 1H), 7.05 (m, 1H), 3.33 (m, 2H), 3.09 (m, 2H), 1.84 (m, 2H), 1.41 (s, 9H). MS m/z 304 (M+1).

### d) 1,1-Dimethylethyl {3-[2-({methyl[(8S)-5,6,7,8-tetrahydro-8-quinolinyl]amino}methyl)imidazo[1,2-a]pyridin-3-yl]propyl}carbamate

Reaction of 1,1-dimethylethyl[3-(2-formylimidazo[1,2-a]pyridin-3-yl)propyl]carbamate (135 mg, 0.44 mmol) and (8S)-N-methyl-5,6,7,8-tetrahydro-8-quinolinamine (72 mg, 0.44 mmol) as described herein for the preparation of (8S)-N-methyl-N-{[3-(4-pyridinyl)imidazo[1,2-a]pyridin-2-yl]methyl}-5,6,7,8-tetrahydro-8-quinolinamine afforded 196 mg (98%) of 1,1-dimethylethyl {3-[2-({methyl[(8S)-5,6,7,8-tetrahydro-8-quinolinyl]amino}methyl)imidazo[1,2-a]pyridin-3-yl]propyl}carbamate as a pale yellow oil. ¹H NMR (DMSO-d₆): δ 8.40 (d, 1H), 8.24 (d, 1H), 7.44 (m, 2H), 7.13 (m, 3H), 6.82 (t, 1H), 3.90-3.76 (m, 3H), 2.94 (m, 3H), 2.75-2.63 (m, 3H), 2.09 (s, 3H), 1.94 (m, 3H), 1.63 (m, 3H), 1.31 (s, 9H). MS m/z 450 (M+1).

### e) (8S)-N-{[3-(3-Aminopropyl)imidazo[1,2-a]pyridin-2-yl]methyl}-N-methyl-5,6,7,8-tetrahydro-8-quinolinamine

A solution of 1,1-dimethylethyl {3-[2-({methyl[(8S)-5,6,7,8-tetrahydro-8-quinolinyl]amino}methyl)imidazo[1,2-a]pyridin-3-yl]propyl}carbamate (194 mg, 0.43 mmol) in 15 mL of 1:2 trifluoroacetic acid/dichloromethane was stirred at rt for 1 h and concentrated. The residue was dissolved in dichloromethane. The solution was washed with 10% aqueous Na₂CO₃ (1×), saturated aqueous brine (1×), dried over Na₂SO₄ and concentrated. Flash chromatography (silica gel, 0 to 10% NH₄OH in acetonitrile) afforded 123 mg (81%) of (8S)-N-{[3-(3-aminopropyl)imidazo[1,2-a]pyridin-2-yl]methyl}-N-methyl-5,6,7,8-tetrahydro-8-quinolinamine as a colorless oil. ¹H NMR (CD₃OD): δ 8.46 (d, 1H), 8.24 (d, 1H), 7.55 (m, 1H), 7.48 (m, 1H), 7.28-7.21 (m, 2H), 6.93 (m, 1H), 4.01 (m, 1H), 3.71 (m, 2H), 3.20-3.03 (m, 2H), 2.89 (m, 1H), 2.81-2.56 (m, 3H), 2.29 (s, 3H), 2.12 (m, 3H), 1.94-1.71 (m, 3H). MS ink 350 (M+1).

### Example 3

### (8S)-N-({3-[3-(Dimethylamino)propyl]imidazo[1,2-a]pyridin-2-yl}methyl)-N-methyl-5,6,7,8-tetrahydro-8-quinolinamine

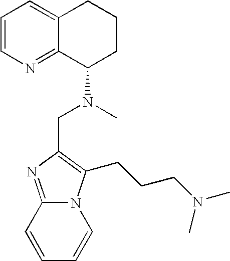

A mixture of (8S)-N-{[3-(3-aminopropyl)imidazo[1,2-a]pyridin-2-yl]methyl}-N-methyl-5,6,7,8-tetrahydro-8-quinolinamine (57 mg, 0.16 mmol), 37% aqueous formaldehyde (36 μL, 0.49 mmol), and NaBH(OAc)₃ (86 mg, 0.41 mmol) in 3 mL of 1,2-dichloroethane was stirred at rt. After 2 h the mixture was diluted with 10% aqueous Na₂CO₃ and brine. After stirring for 15 min, the mixture was filtered through a hydrophobic frit. The aqueous layer was washed with an additional portion of dichloromethane and filtered. The combined filtrates were concentrated. Purification by flash chromatography (silica gel, 0 to 10% NH₄OH in acetonitrile) afforded 48 mg (77%) of (8S)-N-({3-[3-(dimethylamino)propyl]imidazo[1,2-a]pyridin-2-yl}methyl)-N-methyl-5,6,7,8-tetrahydro-8-quinolinamine as a tan oil. ¹H NMR (CD₃OD): δ 8.44 (d, 1H), 8.22 (d, 1H), 7.54 (m, 1H), 7.46 (m, 1H), 7.23 (m, 2H), 6.91 (t, 1H), 3.96 (m, 1H), 3.74 (s, 2H), 3.08-2.73 (m, 4H), 2.31-2.05 (m, 14H), 1.75 (m, 3H). MS m/z 378 (M+H).

### Example 4

### (8S)-N-Methyl-N-[(3-{3-[(2-methylpropyl)amino]propyl}imidazo[1,2-a]pyridin-2-yl)methyl]-5,6,7,8-tetrahydro-8-quinolinamine

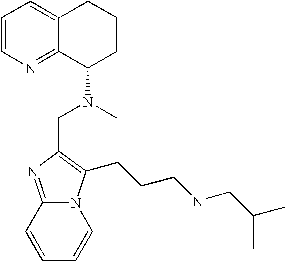

To a solution of (8S)-N-{[3-(3-aminopropyl)imidazo[1,2-a]pyridin-2-yl]methyl}-N-methyl-5,6,7,8-tetrahydro-8-quinolinamine (54 mg, 0.15 mmol) in anhydrous methanol (3 mL) was added isobutyraldehyde (21 μL, 0.23 mmol) and trimethyl orthoformate (51 μL, 0.46 mmol). After stirring at rt for 45 min, the reaction was treated with sodium borohydride (18 mg, 0.46 mmol). After 1 h, the reaction mixture was concentrated under reduced pressure. The residue was taken up in dichloromethane and stirred with 1 N NaOH and brine for 15 min. The mixture was filtered through a hydrophobic frit. The aqueous layer was washed with an additional portion of dichloromethane and filtered. The combined filtrates were concentrated. Flash chromatography (silica gel, 0 to 10% NH₄OH in acetonitrile) afforded 43 mg (68%) of (8S)-N-methyl-N-[(3-{3-[(2-methylpropyl)amino]propyl}imidazo[1,2-a]pyridin-2-yl)methyl]-5,6,7,8-tetrahydro-8-quinolinamine as a pale yellow oil. ¹H NMR (CD₃OD): δ 8.47 (d, 1H), 8.23 (d, 1H), 7.57 (m, 1H), 7.47 (m, 1H), 7.25 (m, 2H), 6.92 (t, 1H), 4.00 (m, 1H), 3.66 (s, 2H), 3.08 (m, 2H), 2.94-2.52 (m, 6H), 2.32 (s, 3H), 2.11 (m, 3H), 1.95 (m, 2H), 1.75 (m, 2H), 0.90 (d, 3H), 0.88 (d, 3H). MS m/z 406 (M+H).

### Example 5

### (8S)-N-Methyl-N-{[3-(3-pyridinyl)imidazo[1,2-a]pyridin-2-yl]methyl}-5,6,7,8-tetrahydro-8-quinolinamine

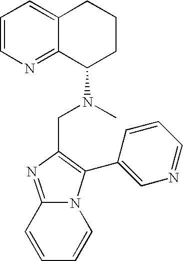

### a) Ethyl 3-(3-pyridinyl)imidazo[1,2-a]pyridine-2-carboxylate

A solution of ethyl 3-iodoimidazo[1,2-a]pyridine-2-carboxylate (314 mg, 0.99 mmol) in 1,2-dimethoxyethane (8 mL) was treated with Pd(PPh₃)₄ (57 mg, 0.05 mmol), 3-pyridineboronic acid (134 mg, 1.09 mmol) and a solution of Na₂CO₃ (210 mg, 1.99 mmol) in 4 mL water. The reaction was heated in a 75° bath for 3 h and another 134 mg 3-pyridineboronic acid was added. After 18 h, the reaction was judged to be complete by LC/MS. After cooling to rt, the reaction was diluted with dichloromethane, then poured into 10% aqueous sodium carbonate. The layers were separated and the aqueous layer was extracted with additional dichloromethane (5×). None of the layers separated cleanly from the emulsion which resulted. The combined organic layers were filtered through hydrophobic frits, dried over Na₂SO₄ and concentrated. Flash chromatography (silica gel, 0 to 4% methanol in dichloromethane) afforded 110 mg (42%) of ethyl 3-(3-pyridinyl)imidazo[1,2-a]pyridine-2-carboxylate as an off-white solid. ¹H NMR (DMSO-d₆): δ 8.73-8.69 (m, 2H), 8.12 (d, 1H), 8.02 (m, 1H), 7.71 (m, 1H), 7.57 (m, 1H), 7.42 (m, 1H), 6.99 (t, 1H), 4.15 (q, 2H), 1.11 (t, 3H). MS m/z 268 (M+1).

### b) [3-(3-Pyridinyl)imidazo[1,2-a]pyridin-2-yl]nethanol

A solution of ethyl 3-(3-pyridinyl)imidazo[1,2-a]pyridine-2-carboxylate (111 mg, 0.41 mmol) in tetrahydrofuran (5 mL) and methanol (2 mL) was cooled to 0°. A solution of lithium borohydride (1.4 mL, 2.0 M in THF) was added dropwise, then the reaction was allowed to warm to rt and stirred for 3 days. The reaction was treated with 1N sodium hydroxide (3 mL) and stirred 15 min before concentrating. The residue was partitioned between brine and ethyl acetate. The aqueous layer was back-extracted with ethyl acetate (4×). The combined organic layers were washed with brine, dried over Na₂SO₄ and concentrated. The resulting foam was purified by flash chromatography (silica gel, 0 to 10% methanol in dichloromethane) to afford 42 mg (45%) of [3-(3-pyridinyl)imidazo[1,2-a]pyridin-2-yl]methanol as a white solid. ¹H NMR (CD₃OD): δ 8.82 (d, 1H), 8.67 (m, 1H), 8.29 (d, 1H), 8.12 (m, 1H), 7.64 (m, 2H), 7.41 (m, 1H), 6.98 (t, 1H), 4.68 (s, 2H).

### c) 3-(3-Pyridinyl)imidazo[1,2-a]pyridine-2-carbaldehyde

Reaction of [3-(3-pyridinyl)imidazo[1,2-a]pyridin-2-yl]methanol (40 mg, 0.18 mmol) as described herein for the preparation of 3-(4-pyridinyl)imidazo[1,2-a]pyridine-2-carbaldehyde afforded 34 mg (85%) of 3-(3-pyridinyl)imidazo[1,2-a]pyridine-2-carbaldehyde as a yellow solid. ¹H NMR (CD₃OD): δ 10.04 (s, 1H), 8.85 (d, 1H), 8.74 (m, 1H), 8.30 (d, 1H), 8.17 (m, 1H), 7.70 (m, 2H), 7.53 (m, 1H), 7.08 (t, 1H). MS m/z 224 (M+1).

### d) (8S)-N-Methyl-N-{[3-(3-pyridinyl)imidazo[1,2-a]pyridin-2-yl]methyl}-5,6,7,8-tetrahydro-8-quinolinamine

Reaction of 3-(3-pyridinyl)imidazo[1,2-a]pyridine-2-carbaldehyde (33 mg, 0.15 mmol) and (8S)-N-methyl-5,6,7,8-tetrahydro-8-quinolinamine (24 mg, 0.15 mmol) as described herein for the preparation of (8S)-N-methyl-N-{[3-(4-pyridinyl)imidazo[1,2-a]pyridin-2-yl]methyl}-5,6,7,8-tetrahydro-8-quinolinamine afforded 39 mg (71%) of (8S)-N-methyl-N-{[3-(3-pyridinyl)imidazo[1,2-a]pyridin-2-yl]methyl}-5,6,7,8-tetrahydro-8-quinolinamine as a tan oil. ¹H NMR (CD₃OD): δ 8.77 (d, 1H), 8.61 (m, 1H), 8.21 (m, 2H), 8.10 (m, 1H), 7.58 (m, 2H), 7.45 (m, 1H), 7.37 (m, 1H), 7.11 (m, 1H), 6.94 (t, 1H), 3.92-3.81 (m, 3H), 2.82-2.64 (m, 2H), 2.27 (s, 3H), 2.07-1.88 (m, 3H), 1.62 (m, 1H). MS m/z 370 (M+1).

### Example 6

### (8S)-N-({3-[(Dimethylamino)methyl]imidazo[1,2-a]pyridin-2-yl}methyl)-N-methyl-5,6,7,8-tetrahydro-8-quinolinamine

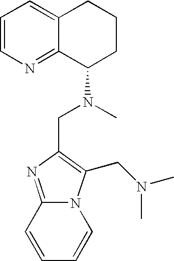

### a) Ethyl 3-cyanoimidazo[1,2-a]pyridine-2-carboxylate

A solution of ethyl 3-iodoimidazo[1,2-a]pyridine-2-carboxylate (4.21 g, 13.3 mmol) in anhydrous NMP (35 mL) was treated with copper(I) cyanide (1.43 g, 16.0 mmol) and heated in a 70° bath. After 18 h, the reaction was cooled to room temperature. It was poured into a solution of disodium EDTA (100 g) in water (600 mL) which had been treated with enough 1 N sodium hydroxide to dissolve all of the solids and still maintain a neutral pH. Ethyl acetate was added and the mixture was stirred for 2 h. The layers were separated, and the aqueous layer was extracted with an additional portion of ethyl acetate. The combined organic layers were washed with brine, dried over Na₂SO₄ and concentrated. Flash chromatography (silica gel, 0 to 100% ethyl acetate in hexanes) to afford 0.91 g (32%) of ethyl 3-cyanoimidazo[1,2-a]pyridine-2-carboxylate as a white solid. ¹H NMR (DMSO-d₆): δ 8.67 (d, 1H), 7.90 (m, 1H), 7.67 (m, 1H), 7.33 (m, 1H), 4.37 (q, 2H), 1.32 (t, 3H). MS m/z 216 (M+1).

### b) 1,1-Dimethylethyl {[2-(hydroxymethyl)imidazo[1,2-a]pyridin-3-yl]methyl}carbamate

A solution of ethyl 3-cyanoimidazo[1,2-a]pyridine-2-carboxylate (859 mg, 3.99 mmol) in 7N NH₃ in MeOH (approx. 100 mL) was hydrogenated over Raney nickel at 45 psi for 1 h. The reaction was filtered through Celite and concentrated. Purification by flash chromatography (silica gel, 0 to 10% methanol in dichloromethane) afforded 0.46 g of a mixture of ethyl 3-(aminomethyl)imidazo[1,2-a]pyridine-2-carboxylate and methyl 3-(aminomethyl)imidazo[1,2-a]pyridine-2-carboxylate as a yellow solid. This mixture was dissolved in dichloromethane (10 mL) and treated with di-t-butyl dicarbonate (0.60 g, 2.72 mmol). After 18 h, the reaction was diluted with dichloromethane and washed with 10% aqueous citric acid (1×), saturated aqueous NaHCO₃ (1×), brine (1×), dried over Na₂SO₄, and concentrated. Flash chromatography (silica gel, 0 to 100% ethyl acetate in hexanes) afforded 509 mg of a 3:1 mixture of methyl 3-[({[(1,1-dimethylethyl)oxy]carbonyl}amino)methyl]imidazo[1,2-a]pyridine-2-carboxylate and ethyl 3-[({[(1,1-dimethylethyl)oxy]carbonyl}amino)methyl]imidazo[1,2-a]pyridine-2-carboxylate as a white foam. MS m/z 306, 320 (M+1). The mixture was dissolved in tetrahydrofuran (6 mL) and methanol (8 mL), cooled to 0° and treated with lithium borohydride (4.6 mL, 2.0 M in THF). The reaction was allowed to warm to it after 20 min, then stirred 3.5 h. After being treated with 1 N sodium hydroxide (5 mL) and stirring 5 min, the reaction was concentrated. The residue was partitioned between ethyl acetate and water. The aqueous layer was back-extracted with ethyl acetate (1×). The combined organic layers were washed with brine, dried over Na₂SO₄ and concentrated to afford 447 mg (40% over 3 steps) of 1,1-dimethylethyl {[2-(hydroxymethyl)imidazo[1,2-a]pyridin-3-yl]methyl}carbamate as a white foam. ¹H NMR (DMSO-d₆): δ 8.36 (d, 1H), 7.47 (m, 1H), 7.32 (m, 1H), 7.20 (m, 1H), 6.90 (t, 1H), 4.83 (m, 1H), 4.58 (d, 2H), 4.50 (d, 2H), 1.33 (s, 9H). MS m/z 278 (M+1).

### c) 1,1-Dimethylethyl[(2-formylimidazo[1,2-a]pyridin-3-yl)methyl]carbamate

Reaction of 1,1-dimethylethyl {[2-(hydroxymethyl)imidazo[1,2-a]pyridin-3-yl]methyl}carbamate (437 mg, 1.58 mmol) as described herein for the preparation of 3-(4-pyridinyl)imidazo[1,2-a]pyridine-2-carbaldehyde afforded 356 mg (82%) of 1,1-dimethylethyl[(2-formylimidazo[1,2-a]pyridin-3-yl)methyl]carbamate as a sticky yellow oil. NMR (DMSO-d₆): δ 10.19 (s, 1H), 8.48 (d, 1H), 7.65 (m, 1H), 7.51 (m, 1H), 7.39 (m, 1H), 7.08 (t, 1H), 4.80 (d, 2H), 1.34 (s, 9H). MS m/z 276 (M+1).

### d) 1,1-Dimethylethyl {[2-({methyl[(8S)-5,6,7,8-tetrahydro-8-quinolinyl]amino}methyl)imidazo[1,2-a]pyridin-3-yl]methyl}carbamate

Reaction of 1,1-dimethylethyl[(2-formylimidazo[1,2-a]pyridin-3-yl)methyl]carbamate (350 mg, 1.27 mmol) and (8S)-N-methyl-5,6,7,8-tetrahydro-8-quinolinamine (206 mg, 1.27 mmol) as described herein for the preparation of (8S)-N-methyl-N-{[3-(4-pyridinyl)imidazo[1,2-a]pyridin-2-yl]methyl}-5,6,7,8-tetrahydro-8-quinolinamine afforded 487 mg (91%) of 1,1-dimethylethyl {[2-({methyl[(8S)-5,6,7,8-tetrahydro-8-quinolinyl]amino}methyl)imidazo[1,2-a]pyridin-3-yl]methyl}carbamate as an off-white foam. ¹H NMR (CD₃OD): δ 8.56 (m, 1H), 8.48 (m, 1H), 7.48 (m, 2H), 7.29 (m, 1H), 7.13 (m, 1H), 6.94 (m, 1H), 4.72-4.56 (m, 2H), 3.96 (broad, 3H), 2.87-2.65 (m, 3H), 2.17-2.02 (m, 5H), 1.70 (m, 1H), 1.42 (s, 9H). MS m/z 422 (M+1).

### e) (8S)-N-{[3-(Aminomethyl)imidazo[1,2-a]pyridin-2-yl]methyl}-N-methyl-5,6,7,8-tetrahydro-8-quinolinamine

Reaction of 1,1-dimethylethyl{[2-({methyl[(8S)-5,6,7,8-tetrahydro-8-quinolinyl]amino}methyl)imidazo[1,2-a]pyridin-3-yl]methyl}carbamate (483 mg, 1.15 mmol) as described herein for the preparation of (8S)-N-{[3-(3-aminopropyl)imidazo[1,2-a]pyridin-2-yl]methyl}-N-methyl-5,6,7,8-tetrahydro-8-quinolinamine afforded 313 mg (85%) of (8S)-N-{[3-(aminomethyl)imidazo[1,2-a]pyridin-2-yl]methyl}-N-methyl-5,6,7,8-tetrahydro-8-quinolinamine as a yellow oil. ¹H NMR (CD₃OD): δ 8.44 (d, 1H), 8.40 (m, 1H), 7.53 (m, 2H), 7.35 (m, 1H), 7.18 (m, 1H), 7.02 (t, 1H), 4.40 (s, 2H), 4.06-3.89 (m, 3H), 2.92-2.74 (m, 2H), 2.25-1.98 (m, 6H), 1.73 (m, 1H). MS m/z 322 (M+1).

### f) (8S)-N-({3-[(Dimethylamino)methyl]imidazo[1,2-a]pyridin-2-yl}methyl)-N-methyl-5,6,7,8-tetrahydro-8-quinolinamine

Reaction of (8S)-N-{[3-(aminomethyl)imidazo[1,2-a]pyridin-2-yl]methyl}-N-methyl-5,6,7,8-tetrahydro-8-quinolinamine (51.2 mg, 0.16 mmol) as described herein for the preparation of (8S)-N-({3-[3-(dimethylamino)propyl]imidazo[1,2-a]pyridin-2-yl}methyl)-N-methyl-5,6,7,8-tetrahydro-8-quinolinamine afforded 27.9 mg (50%) of (8S)-N-({3-[(dimethylamino)methyl]imidazo[1,2-a]pyridin-2-yl}methyl)-N-methyl-5,6,7,8-tetrahydro-8-quinolinamine as a pale yellow oil after a second purification by reverse phase HPLC (C8, 0 to 100% acetonitrile in water containing 0.1% TFA), followed by free basing with dichloromethane and 10% aqueous sodium carbonate. ¹H NMR (DMSO-d₆): δ 8.40 (d, 1H), 8.27 (d, 1H), 7.45 (m, 2H), 7.15 (m, 2H), 6.83 (t, 1H), 3.92-3.60 (m, 5H), 2.83-2.63 (m, 2H), 2.10 (s, 3H), 2.08-1.82 (m, 9H), 1.61 (m, 1H). MS m/z 350 (M+H).

### Example 7

### N²,N²-Dimethyl-N¹-{[2-({methyl[(8S)-5,6,7,8-tetrahydro-8-quinolinyl]amino}methyl)imidazo[1,2-a]pyridin-3-yl]methyl}glycinamide

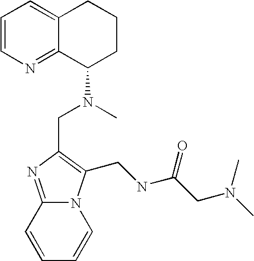

### a) 1,1-Dimethylethyl[2-({[2-({methyl[(8S)-5,6,7,8-tetrahydro-8-quinolinyl]amino}methyl)imidazo[1,2-a]pyridin-3-yl]methyl}amino)-2-oxoethyl]carbamate

A solution of (8S)-N-{[3-(aminomethyl)imidazo[1,2-a]pyridin-2-yl]methyl}-N-methyl-5,6,7,8-tetrahydro-8-quinolinamine (170 mg, 0.53 mmol) in anhydrous acetonitrile (15 mL) was treated with Boc-glycine (139 mg, 0.79 mmol), DIEA (0.28 mL, 1.59 mmol) and HATU (0.40 g, 1.06 mmol). After stirring for 18 h, the reaction was concentrated. The residue was dissolved in ethyl acetate and washed with 10% aqueous sodium carbonate and brine, then dried over Na₂SO₄. Flash chromatography (silica gel, 0 to 10% NH₄OH in acetonitrile) afforded 192 mg (76%) of 1,1-dimethylethyl[2-({[2-({methyl[(8S)-5,6,7,8-tetrahydro-8-quinolinyl]amino}methyl)imidazo[1,2-a]pyridin-3-yl]methyl}amino)-2-oxoethyl]carbamate as a yellow oil. ¹H NMR (DMSO-d₆): δ 8.99 (m, 1H), 8.49 (m, 2H), 7.49 (m, 2H), 7.18 (m, 2H), 6.88 (m, 2H), 4.80-4.59 (m, 2H), 3.93 (m, 3H), 3.52 (m, 2H), 2.77-2.65 (m, 3H), 2.00 (m, 5H), 1.60 (m, 1H), 1.30 (s, 9H). MS m/z 479 (M+H).

### b) N¹-[(2-{[Methyl(5,6,7,8-tetrahydro-8-quinolinyl)amino]methyl}imidazo[1,2-a]pyridin-3-yl)methyl]glycinamide

A solution of 1,1-dimethylethyl[2-({[2-({methyl[(8S)-5,6,7,8-tetrahydro-8-quinolinyl]amino}methyl)imidazo[1,2-a]pyridin-3-yl]methyl}amino)-2-oxoethyl]carbamate (190 mg, 0.40 mmol) in dichloromethane (4 mL) was treated with trifluoroacetic acid (2 mL) and allowed to stir at room temperature for 45 min. The reaction was concentrated and the residue was partitioned between dichloromethane and 10% aqueous sodium carbonate. The aqueous layer was back-extracted with dichloromethane. The combined organic layers were washed with brine and dried over Na₂SO₄ to afford 116 mg (77%) of N¹-[(2-{[methyl(5,6,7,8-tetrahydro-8-quinolinyl)amino]methyl}imidazo[1,2-a]pyridin-3-yl)methyl]glycinamide as an off-white foam. ¹H NMR (DMSO-d₆): δ 8.77 (m, 1H), 8.54 (d, 1H), 8.46 (d, 1H), 7.48 (m, 2H), 7.17 (m, 2H), 6.89 (t, 1H), 4.81-4.66 (m, 2H), 3.94 (m, 3H), 3.08 (s, 2H), 2.81-2.65 (m, 2H), 2.03-1.61 (m, 9H). MS m/z 379 (M+H).

### c) N²,N²-Dimethyl-N¹-{[2-({methyl[(8S)-5,6,7,8-tetrahydro-8-quinolinyl]amino}methyl)imidazo[1,2-a]pyridin-3-yl]methyl}glycinamide

A mixture of N¹-[(2-{[methyl(5,6,7,8-tetrahydro-8-quinolinyl)amino]methyl}imidazo[1,2-a]pyridin-3-yl)methyl]glycinamide (101 mg, 0.27 mmol), 37% aqueous formaldehyde (60 μL, 0.80 mmol), and NaBH(OAc)₃ (141 mg, 0.67 mmol) in 10 mL of 1,2-dichloroethane was stirred at rt. After 2.5 h the mixture was diluted with 10% aqueous Na₂CO₃ and extracted with dichloromethane (2×). The combined organic layers were washed with brine and dried over Na₂SO₄. Purification by reverse phase HPLC(C8, 0 to 50% acetonitrile in water containing 0.1% TFA), followed by free basing with dichloromethane and 10% aqueous sodium carbonate afforded 84 mg (78%) of N²,N²-dimethyl-N¹-{[2-({methyl[(8S)-5,6,7,8-tetrahydro-8-quinolinyl]amino}methyl)imidazo[1,2-a]pyridin-3-yl]methyl}glycinamide as a white foam. ¹H NMR (DMSO-d₆): δ 8.64 (t, 1H), 8.54 (d, 1H), 8.44 (d, 1H), 7.46 (m, 2H), 7.15 (m, 2H), 6.88 (t, 1H), 4.79-4.63 (m, 2H), 3.93 (m, 3H), 2.89-2.64 (m, 4H), 2.02 (s, 9H), 1.90 (m, 3H), 1.59 (m, 1H). MS m/z 407 (M+H).

### Example 8

### (8S)-N-Methyl-N-({3-[6-(4-morpholinyl)-3-pyridinyl]imidazo[1,2-a]pyridin-2-yl}methyl)-5,6,7,8-tetrahydro-8-quinolinamine

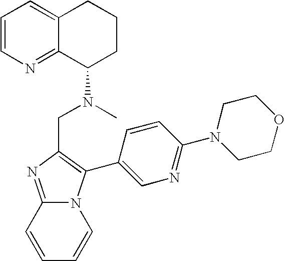

### a) Ethyl 3-(6-fluoro-3-pyridinyl)imidazo[1,2-a]pyridine-2-carboxylate

A solution of ethyl 3-iodoimidazo[1,2-a]pyridine-2-carboxylate (1.01 g, 3.20 mmol) in 1,4-dioxane (15 mL) was treated with Pd(PPh₃)₄ (185 mg, 0.16 mmol), (6-fluoro-3-pyridinyl)boronic acid (495 mg, 3.51 mmol) and a solution of Na₂CO₃ (677 mg, 6.39 mmol) in 5 mL water. The reaction was heated in a 90° bath for 5 h and cooled to rt. The mixture was poured into water and extracted with ethyl acetate (3×). The combined organic layers were washed with brine, dried over Na₂SO₄ and concentrated. Flash chromatography (silica gel, 0 to 6% methanol in dichloromethane) afforded 0.75 g (82%) of ethyl 3-(6-fluoro-3-pyridinyl)imidazo[1,2-a]pyridine-2-carboxylate as a pale yellow solid. ¹H NMR (DMSO-d₆): δ 8.44 (m, 1H), 8.22 (m, 1H), 8.15 (d, 1H), 7.71 (d, 1H), 7.40 (m, 2H), 6.99 (t, 1H), 4.16 (q, 2H), 1.13 (t, 3H). MS m/z 286 (M+1).

### b) Ethyl 3-[6-(4-morpholinyl)-3-pyridinyl]imidazo[1,2-a]pyridine-2-carboxylate

A mixture of ethyl 3-(6-fluoro-3-pyridinyl)imidazo[1,2-a]pyridine-2-carboxylate (248 mg, 0.87 mmol) and morpholine (10 mL) was heated in a 90° bath. After heating for 18 h, the reaction was cooled to room temperature and concentrated to dryness. Flash chromatography (silica gel, 0 to 6% methanol in dichloromethane) afforded 0.26 g (85%) of ethyl 3-[6-(4-morpholinyl)-3-pyridinyl]imidazo[1,2-a]pyridine-2-carboxylate as a white solid. ¹H NMR (DMSO-d₆): δ 8.25 (d, 1H), 8.11 (d, 1H), 7.70 (m, 2H), 7.36 (m, 1H), 6.96 (m, 2H), 4.16 (q, 2H), 3.70 (m, 4H), 3.54 (m, 4H), 1.16 (t, 3H). MS m/z 353 (M+1).

### c) {3-[6-(4-Morpholinyl)-3-pyridinyl]imidazo[1,2-a]pyridin-2-yl}methanol

A solution of ethyl 3-[6-(4-morpholinyl)-3-pyridinyl]imidazo[1,2-a]pyridine-2-carboxylate (0.26 g, 0.74 mmol) in 1:1 methanol/THF (10 mL) was treated with lithium borohydride (1.50 mL, 2.0 M in THF) and allowed to stir at room temperature. After 1 h, another 1.50 mL lithium borohydride was added. After 19 h, the reaction was treated with 1 N sodium hydroxide (5 mL) and stirred 5 min, then concentrated. The residue was partitioned between ethyl acetate and water. The aqueous layer was back-extracted with ethyl acetate (3×). The combined organic layers were washed with brine, dried over Na₂SO₄ and concentrated. Flash chromatography (silica gel, 0 to 10% methanol in dichloromethane) afforded 142 mg (62%) of {3-[6-(4-morpholinyl)-3-pyridinyl]imidazo[1,2-a]pyridin-2-yl}methanol as a white solid. ¹H NMR (DMSO-d₆): δ 8.32 (m, 1H), 8.20 (m, 1H), 7.76 (m, 1H), 7.53 (m, 1H), 7.23 (m, 1H), 6.99 (m, 1H), 6.85 (m, 1H), 5.05 (m, 1H), 4.43 (m, 2H), 3.70 (m, 4H), 3.52 (m, 4H). MS m/z 311 (M+1).

### d) 3-[6-(4-Morpholinyl)-3-pyridinyl]imidazo[1,2-a]pyridine-2-carbaldehyde

A solution of {3-[6-(4-morpholinyl)-3-pyridinyl]imidazo[1,2-a]pyridin-2-yl}methanol (94 mg, 0.30 mmol) in dichloromethane (10 mL) was treated with IBX polystyrene resin (0.55 g, 1.1 mmol/g, Novabiochem) and allowed to stir at RT for 6 h. The resin was removed by filtration and the reaction mixture was concentrated to afford 70 mg (75%) of 3-[6-(4-morpholinyl)-3-pyridinyl]imidazo[1,2-a]pyridine-2-carbaldehyde as a tan crystalline solid. ¹H NMR (DMSO-d₆): δ 9.94 (s, 1H), 8.36 (d, 1H), 8.27 (d, 1H), 7.85 (d, 1H), 7.68 (m, 1H), 7.40 (m, 1H), 6.99 (m, 2H), 3.71 (m, 4H), 3.57 (m, 4H). MS m/z 309 (M+1).

### e) (8S)-N-Methyl-N-({3-[6-(4-morpholinyl)-3-pyridinyl]imidazo[1,2-a]pyridin-2-yl}methyl)-5,6,7,8-tetrahydro-8-quinolinamine

Reaction of 3-[6-(4-morpholinyl)-3-pyridinyl]imidazo[1,2-a]pyridine-2-carbaldehyde (70 mg, 0.23 mmol) and (8S)-N-methyl-5,6,7,8-tetrahydro-8-quinolinamine (37 mg, 0.23 mmol) as described herein for the preparation of (8S)-N-methyl-N-{[3-(4-pyridinyl)imidazo[1,2-a]pyridin-2-yl]methyl}-5,6,7,8-tetrahydro-8-quinolinamine afforded 88 mg (85%) of (8S)-N-methyl-N-({3-[6-(4-morpholinyl)-3-pyridinyl]imidazo[1,2-a]pyridin-2-yl}methyl)-5,6,7,8-tetrahydro-8-quinolinamine as a pale yellow oil after a second purification by reverse phase HPLC(C8, 0 to 100% acetonitrile in water containing 0.1% TFA), followed by free basing with dichloromethane and 10% aqueous sodium carbonate. ¹H NMR (DMSO-d₆): δ 8.31 (m, 2H), 8.16 (d, 1H), 7.84 (m, 1H), 7.53 (m, 1H), 7.41 (m, 1H), 7.19 (m, 1H), 7.08 (m, 1H), 6.92 (d, 1H), 6.82 (t, 1H), 3.87-3.76 (m, 3H), 3.70 (m, 4H), 3.51 (m, 4H), 2.76-2.57 (m, 2H), 2.08 (s, 3H), 1.91-1.78 (m, 3H), 1.54 (m, 1H). MS m/z 455 (M+1).

### Example 9

### (8S)-N-Methyl-N-{[3-(1-pyrrolidinylmethyl)imidazo[1,2-a]pyridin-2-yl]methyl}-5,6,7,8-tetrahydro-8-quinolinamine

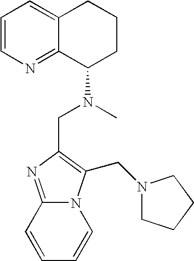

### a) Imidazo[1,2-a]pyridin-2-ylmethanol

A solution of ethyl imidazo[1,2-a]pyridine-2-carboxylate (2.01 g, 10.6 mmol) in 20 mL of a 1:1 mixture of THF and methanol was treated with lithium borohydride (15.9 mL, 2.0 M in THF). After 1 h, another 15.9 mL lithium borohydride solution was added. The reaction was stirred for another 2.5 h, then treated with 10 mL of 1N sodium hydroxide. After stirring 5 min, the reaction was concentrated to remove most of the organic solvents. The mixture was diluted with brine and 10% aqueous sodium carbonate, then extracted with a 3:1 mixture of chloroform and isopropanol (3×). The combined organic layers were dried over Na₂SO₄ and concentrated. Hexane was added and the mixture was concentrated. Purification by flash chromatography (silica gel, 0 to 10% methanol in dichloromethane) afforded 1.25 g (80%) of imidazo[1,2-a]pyridin-2-ylmethanol as a white solid. ¹H NMR (CDCl₃): δ 8.07 (d, 1H), 7.55 (m, 2H), 7.16 (m, 1H), 6.76 (t, 1H), 4.84 (s, 2H), 2.72 (broad, 1H).

### b) Imidazo[1,2-a]pyridine-2-carbaldehyde

A solution of imidazo[1,2-a]pyridin-2-ylmethanol (0.64 g, 4.32 mmol) in 1:1 acetonitrile/chloroform (30 mL) was treated with manganese dioxide (4.51 g, 51.8 mmol) and stirred for 4 h. Another 0.37 g (4.32 mmol) manganese dioxide was added and the reaction was stirred 18 h. The mixture was filtered through a pad of Celite and washed thoroughly with chloroform and acetonitrile. The filtrate was concentrated to afford 0.35 g (51%) of imidazo[1,2-a]pyridine-2-carbaldehyde as an off-white solid. ¹H NMR (DMSO-d₆): δ 10.01 (s, 1H), 8.60 (m, 2H), 7.64 (m, 1H), 7.36 (m, 1H), 7.01 (t, 1H). MS m/z 147 (M+1).

### c) (8S)-N-(Imidazo[1,2-a]pyridin-2-ylmethyl)-N-methyl-5,6,7,8-tetrahydro-8-quinolinamine

Reaction of imidazo[1,2-a]pyridine-2-carbaldehyde (0.65 g, 4.45 mmol) and (8S)-N-methyl-5,6,7,8-tetrahydro-8-quinolinamine (0.72 g, 4.45 mmol) as described herein for the preparation of (8S)-N-methyl-N-{[3-(4-pyridinyl)imidazo[1,2-a]pyridin-2-yl]methyl}-5,6,7,8-tetrahydro-8-quinolinamine afforded a quantitative yield of (8S)-N-(imidazo[1,2-a]pyridin-2-ylmethyl)-N-methyl-5,6,7,8-tetrahydro-8-quinolinamine as a reddish-brown oil. ¹H NMR (DMSO-d₆): δ 8.45 (d, 1H), 8.37 (m, 1H), 7.78 (s, 1H), 7.43 (m, 2H), 7.12 (m, 2H), 6.78 (t, 1H), 3.88 (m, 3H), 2.82-2.61 (m, 2H), 2.20 (s, 3H), 1.93 (m, 3H), 1.60 (m, 1H). MS m/z 293 (M+1).

### d) (8S)-N-Methyl-N-{[3-(1-pyrrolidinylmethyl)imidazo[1,2-a]pyridin-2-yl]methyl}-5,6,7,8-tetrahydro-8-quinolinamine

A mixture of (8S)-N-(imidazo[1,2-a]pyridin-2-ylmethyl)-N-methyl-5,6,7,8-tetrahydro-8-quinolinamine (63 mg, 0.22 mmol), pyrrolidine (0.18 mL, 2.15 mmol), formalin (0.16 mL, 37% aqueous solution), water (2 mL) and glacial acetic acid (2 mL) was heated in a 50° bath. After 18 h, the reaction was cooled to room temperature and concentrated. The residue was diluted with 10% aqueous sodium carbonate and extracted with ethyl acetate (2×). The combined organic layers were washed with brine, dried over Na₂SO₄ and concentrated. Purification by flash chromatography (silica gel, 0 to 10% NH₄OH in acetonitrile) afforded 59 mg (73%) of (8S)-N-methyl-N-{[3-(1-pyrrolidinylmethyl)imidazo[1,2-a]pyridin-2-yl]methyl}-5,6,7,8-tetrahydro-8-quinolinamine as a pale yellow oil. ¹H NMR (DMSO-d₆): δ 8.40-8.32 (m, 2H), 7.46 (m, 2H), 7.16 (m, 2H), 6.84 (m, 1H), 3.95-3.76 (m, 5H), 3.30 (broad, 4H), 2.83-2.64 (m, 2H), 2.32 (broad, 4H), 2.11 (s, 3H), 2.04-1.87 (m, 3H), 1.61 (m, 1H). MS m/z 376 (M+1).

### Example 10

### (8S)-N-Methyl-N-{[3-(4-morpholinylmethyl)imidazo[1,2-a]pyridin-2-yl]methyl}-5,6,7,8-tetrahydro-8-quinolinamine

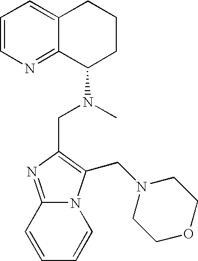

A mixture of (8S)-N-(imidazo[1,2-a]pyridin-2-ylmethyl)-N-methyl-5,6,7,8-tetrahydro-8-quinolinamine (57 mg, 0.19 mmol), morpholine (0.17 mL, 1.95 mmol), formalin (0.145 mL, 37% aqueous solution), and glacial acetic acid (3 mL) was heated in a 50° bath. After 18 h, the reaction was cooled to room temperature and concentrated. The residue was diluted with 10% aqueous sodium carbonate and extracted with ethyl acetate (2×). The combined organic layers were washed with brine, dried over Na₂SO₄ and concentrated. Purification by flash chromatography (silica gel, 0 to 10% NH₄OH in acetonitrile) afforded 65 mg (86%) of (8S)-N-methyl-N-{[3-(4-morpholinylmethyl)imidazo[1,2-a]pyridin-2-yl]methyl}-5,6,7,8-tetrahydro-8-quinolinamine as a yellow oil. ¹H NMR (DMSO-d₆): δ 8.41-8.34 (m, 2H), 7.46 (m, 2H), 7.17 (m, 2H), 6.86 (t, 1H), 3.94-3.73 (m, 5H), 3.53-3.45 (m, 5H), 2.83-2.64 (m, 2H), 2.26 (m, 4H), 2.11 (s, 3H), 1.96 (m, 2H), 1.62 (m, 1H). MS m/z 392 (M+1).

### Example 11

### (8S)-N-({3-[6-(Dimethylamino)-3-pyridinyl]imidazo[1,2-a]pyridin-2-yl}methyl)-N-methyl-5,6,7,8-tetrahydro-8-quinolinamine

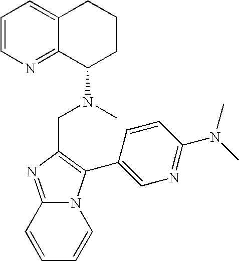

### a) Ethyl 3-[6-(dimethylamino)-3-pyridinyl]imidazo[1,2-a]pyridine-2-carboxylate

A mixture of ethyl 3-(6-fluoro-3-pyridinyl)imidazo[1,2-a]pyridine-2-carboxylate (194 mg, 0.68 mmol) and dimethylamine (5 mL, 2.0 M in THF) was heated in a sealed tube in a 70° bath. After heating for 18 h, the temperature was increased to 80° for 2 h. The reaction was cooled to room temperature and concentrated to dryness. Flash chromatography (silica gel, 0 to 10% methanol in dichloromethane) afforded 205 mg (97%) of ethyl 3-[6-(dimethylamino)-3-pyridinyl]imidazo[1,2-a]pyridine-2-carboxylate as a light yellow oil which partially solidified upon standing. ¹H NMR (DMSO-d₆): δ 8.19 (d, 1H), 8.09 (d, 1H), 7.65-7.50 (m, 2H), 7.35 (m, 1H), 6.93 (t, 1H), 6.76 (d, 1H), 4.16 (q, 2H), 3.08 (s, 6H), 1.16 (t, 3H). MS m/z 311 (M+1).

### b) {3-[6-(Dimethylamino)-3-pyridinyl]imidazo[1,2-a]pyridin-2-yl}methanol

A solution of ethyl 3-[6-(dimethylamino)-3-pyridinyl]imidazo[1,2-a]pyridine-2-carboxylate (201 mg, 0.65 mmol) in 10 mL methanol was treated with lithium borohydride (2.6 mL, 2.0 M in THF) and allowed to stir at room temperature. Two additional 2.6 mL portions of lithium borohydride were added at 2 h intervals and the reaction was allowed to stir at room temperature overnight, after the addition of 5 mL of methanol. Three additional 2.6 mL portions of lithium borohydride were added the next day, with the addition of enough methanol (a total of 15 mL) to keep the reaction mixture stirring. After another 18 h, the reaction was treated with 1 N sodium hydroxide (5 mL) and stirred 15 min, then concentrated. The residue was diluted with water and extracted with ethyl acetate (4×). The combined organic layers were washed with brine, dried over Na₂SO₄ and concentrated. Flash chromatography (silica gel, 0 to 10% methanol in dichloromethane) afforded 58 mg (33%) of {3-[6-(dimethylamino)-3-pyridinyl]imidazo[1,2-a]pyridin-2-yl}methanol as a white solid. ¹H NMR (DMSO-d₆): δ 8.26 (d, 1H), 8.18 (d, 1H), 7.69 (m, 1H), 7.53 (m, 1H), 7.22 (m, 1H), 6.86-6.77 (m, 2H), 5.03 (t, 1H), 4.43 (d, 2H), 3.08 (s, 6H). MS m/z 269 (M+1).

### c) 3-[6-(Dimethylamino)-3-pyridinyl]imidazo[1,2-a]pyridine-2-carbaldehyde

A solution of {3-[6-(dimethylamino)-3-pyridinyl]imidazo[1,2-a]pyridin-2-yl}methanol (47 mg, 0.18 mmol) in chloroform (15 mL) was treated with IBX polystyrene resin (318 mg, 1.1 mmol/g, Novabiochem) and allowed to stir at RT for 18 h. The resin was removed by filtration and the reaction mixture was concentrated to afford 27 mg (57%) of 3-[6-(dimethylamino)-3-pyridinyl]imidazo[1,2-a]pyridine-2-carbaldehyde as a yellow solid. ¹H NMR (DMSO-d₆): δ 9.94 (s, 1H), 8.33 (d, 1H), 8.26 (d, 1H), 7.78 (m, 1H), 7.68 (m, 1H), 7.41 (m, 1H), 6.99 (t, 1H), 6.82 (d, 1H), 3.12 (s, 6H). MS m/z 267 (M+1).

### d) (8S)-N-({3-[6-(Dimethylamino)-3-pyridinyl]imidazo[1,2-a]pyridin-2-yl}methyl)-N-methyl-5,6,7,8-tetrahydro-8-quinolinamine

Reaction of 3-[6-(dimethylamino)-3-pyridinyl]imidazo[1,2-a]pyridine-2-carbaldehyde (30 mg, 0.11 mmol) and (8S)-N-methyl-5,6,7,8-tetrahydro-8-quinolinamine (18 mg, 0.11 mmol) as described herein for the preparation of (8S)-N-methyl-N-{[3-(4-pyridinyl)imidazo[1,2-a]pyridin-2-yl]methyl}-5,6,7,8-tetrahydro-8-quinolinamine afforded a 42 mg (91%) of (8S)-N-({3-[6-(dimethylamino)-3-pyridinyl]imidazo[1,2-a]pyridin-2-yl}methyl)-N-methyl-5,6,7,8-tetrahydro-8-quinolinamine as a yellow oil after reverse phase HPLC purification (C8, 0 to 100% acetonitrile in water containing 0.1% TFA), followed by free basing with dichloromethane and 10% aqueous sodium carbonate. ¹H NMR (DMSO-d₆): δ 8.32 (d, 1H), 8.26 (d, 1H), 8.13 (d, 1H), 7.76 (m, 1H), 7.53 (d, 1H), 7.42 (d, 1H), 7.19 (m, 1H), 7.10 (m, 1H), 6.82 (t, 1H), 6.73 (d, 1H), 3.87-3.76 (m, 3H), 3.09 (s, 6H), 2.76-2.59 (m, 2H), 2.10 (s, 3H), 1.93-1.79 (m, 3H), 1.54 (m, 1H). MS m/z 413 (M+1).

### Example 12

### (8S)-N-Methyl-N-{[3-(1-piperidinylmethyl)imidazo[1,2-a]pyridin-2-yl]methyl}-5,6,7,8-tetrahydro-8-quinolinamine

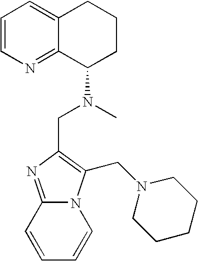

Reaction of (8S)-N-(imidazo[1,2-a]pyridin-2-ylmethyl)-N-methyl-5,6,7,8-tetrahydro-8-quinolinamine (63 mg, 0.22 mmol) and piperidine (0.21 mL, 2.15 mmol) as described herein for the preparation of (8S)-N-methyl-N-{[3-(4-morpholinylmethyl)imidazo[1,2-a]pyridin-2-yl]methyl}-5,6,7,8-tetrahydro-8-quinolinamine afforded 61 mg (73%) of (8S)-N-methyl-N-{[3-(1-piperidinylmethyl)imidazo[1,2-a]pyridin-2-yl]methyl}-5,6,7,8-tetrahydro-8-quinolinamine as a pale gold oil. ¹H NMR (DMSO-d₆): δ 8.40 (d, 1H), 8.31 (d, 1H), 7.46 (m, 2H), 7.16 (m, 2H), 6.85 (t, 1H), 3.93-3.65 (m, 5H), 2.83-2.64 (m, 2H), 2.20 (m, 4H), 2.11 (s, 3H), 2.04-1.83 (m, 3H), 1.61 (m, 1H), 1.36 (m, 6H). MS m/z 390 (M+1).

### Example 13

### (8S)-N-[(3-{[3-Dimethylamino-1-pyrrolidinyl]methyl}imidazo[1,2-a]pyridin-2-yl)methyl]-N-methyl-5,6,7,8-tetrahydro-8-quinolinamine

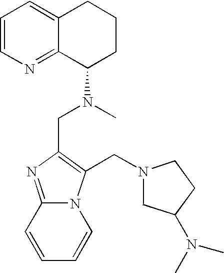

Reaction of (8S)-N-(imidazo[1,2-a]pyridin-2-ylmethyl)-N-methyl-5,6,7,8-tetrahydro-8-quinolinamine (63 mg, 0.21 mmol) and N,N-dimethyl-3-pyrrolidinamine (0.27 mL, 2.15 mmol) as described herein for the preparation of (8S)-N-methyl-N-{[3-(4-morpholinylmethyl)imidazo[1,2-a]pyridin-2-yl]methyl}-5,6,7,8-tetrahydro-8-quinolinamine afforded 47 mg (52%) of (8S)-N-[(3-{[3-(dimethylamino)-1-pyrrolidinyl]methyl}imidazo[1,2-a]pyridin-2-yl)methyl]-N-methyl-5,6,7,8-tetrahydro-8-quinolinamine as a pale yellow oil. ¹H NMR (DMSO-d₆): δ 8.39 (d, 1H), 8.30 (d, 1H), 7.46 (m, 2H), 7.15 (m, 2H), 6.83 (t, 1H), 3.94-3.76 (m, 5H), 2.82-2.50 (m, 4H), 2.43-2.33 (m, 2H), 2.18 (m, 1H), 2.11 (s, 3H), 1.98 (m, 8H), 1.88-1.48 (m, 4H). MS m/z 419 (M+1).

### Example 14

### (8S)-N-Methyl-N-({3-[6-(4-methyl-1-piperazinyl)-3-pyridinyl]imidazo[1,2-a]pyridin-2-yl}methyl)-5,6,7,8-tetrahydro-8-quinolinamine

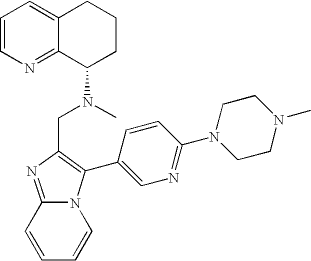

### a) Ethyl 3-[6-(4-methyl-1-piperazinyl)-3-pyridinyl]imidazo[1,2-a]pyridine-2-carboxylate

A solution of ethyl 3-(6-fluoro-3-pyridinyl)imidazo[1,2-a]pyridine-2-carboxylate (169 mg, 0.59 mmol) and 1-methylpiperazine (0.66 mL, 5.92 mmol) in 1,4-dioxane (3 mL) was heated in a 50° bath. After heating for 20 h, the temperature was gradually increased to 80° over 8 h, then the reaction was cooled to room temperature and concentrated to dryness. Flash chromatography (silica gel, 0 to 10% methanol in dichloromethane) afforded 124 mg (57%) of ethyl 3-[6-(4-methyl-1-piperazinyl)-3-pyridinyl]imidazo[1,2-a]pyridine-2-carboxylate as a colorless oil. ¹H NMR (DMSO-d₆): δ 8.22 (d, 1H), 8.11 (d, 1H), 7.66 (m, 2H), 7.35 (m, 1H), 6.94 (m, 2H), 4.16 (q, 2H), 3.57 (m, 4H), 2.40 (m, 4H), 2.21 (s, 3H), 1.16 (t, 3H). MS m/z 366 (M+1).

### b) {3-[6-(4-Methyl-1-piperazinyl)-3-pyridinyl]imidazo[1,2-a]pyridin-2-yl}methanol

A solution of ethyl 3-[6-(4-methyl-1-piperazinyl)-3-pyridinyl]imidazo[1,2-a]pyridine-2-carboxylate (123 mg, 0.34 mmol) in 10 mL methanol was treated with lithium borohydride (0.67 mL, 2.0 M in THF) and allowed to stir at room temperature. After 1 h, another 0.67 mL portion of lithium borohydride was added, followed by 1.3 mL 2 h later. The reaction was allowed to stir at room temperature overnight, and 3 additional 1.7 mL portions of lithium borohydride were added over 5 h. After another 1 h, the reaction was treated with 1 N sodium hydroxide (5 mL) and stirred 15 min, then concentrated. The residue was diluted with water and extracted with ethyl acetate (4×). The combined organic layers were washed with brine, dried over Na₂SO₄ and concentrated. Flash chromatography (silica gel, 0 to 10% methanol in dichloromethane) afforded 25 mg (23%) of {3-[6-(4-methyl-1-piperazinyl)-3-pyridinyl]imidazo[1,2-a]pyridin-2-yl}methanol as an opaque white oil. ¹H NMR (DMSO-d₆): δ 8.34 (d, 1H), 8.20 (d, 1H), 7.79 (m, 1H), 7.54 (m, 1H), 7.24 (m, 1H), 7.03 (d, 1H), 6.86 (t, 1H), 5.06 (t, 1H), 4.44 (d, 2H), 3.84-3.69 (m, 4H), 2.98-2.84 (m, 4H), 2.61 (s, 3H).

### c) 3-[6-(4-Methyl-1-piperazinyl)-3-pyridinyl]imidazo[1,2-a]pyridine-2-carbaldehyde

A solution of {3-[6-(4-methyl-1-piperazinyl)-3-pyridinyl]imidazo[1,2-a]pyridin-2-yl}methanol (21 mg, 0.065 mmol) in chloroform (10 mL) was treated with manganese dioxide (56 mg, 0.65 mmol) and stirred overnight. The reaction mixture was filtered through Celite and concentrated to afford 15 mg (71%) of 3-[6-(4-methyl-1-piperazinyl)-3-pyridinyl]imidazo[1,2-a]pyridine-2-carbaldehyde as a yellow solid. ¹H NMR (CDCl₃): δ 10.13 (s, 1H), 8.37 (s, 1H), 8.07 (d, 1H), 7.70 (m, 2H), 7.30 (m, 1H), 6.84 (m, 2H), 3.92 (m, 4H), 3.18 (m, 2H), 2.84 (m, 2H), 2.73 (s, 3H).

### d) (8S)-N-Methyl-N-({3-[6-(4-methyl-1-piperazinyl)-3-pyridinyl]imidazo[1,2-a]pyridin-2-yl}methyl)-5,6,7,8-tetrahydro-8-quinolinamine

Reaction of 3-[6-(4-methyl-1-piperazinyl)-3-pyridinyl]imidazo[1,2-a]pyridine-2-carbaldehyde (15 mg, 0.047 mmol) and (8S)-N-methyl-5,6,7,8-tetrahydro-8-quinolinamine (8 mg, 0.047 mmol) as described herein for the preparation of (8S)-N-methyl-N-{[3-(4-pyridinyl)imidazo[1,2-a]pyridin-2-yl]methyl}-5,6,7,8-tetrahydro-8-quinolinamine afforded 18 mg (82%) of (8S)-N-methyl-N-({3-[6-(4-methyl-1-piperazinyl)-3-pyridinyl]imidazo[1,2-a]pyridin-2-yl}methyl)-5,6,7,8-tetrahydro-8-quinolinamine as a pale yellow oil. ¹H NMR (DMSO-d₆): δ 8.30 (m, 2H), 8.15 (d, 1H), 7.79 (m, 1H), 7.53-7.40 (m, 2H), 7.21-7.07 (m, 2H), 6.92-6.80 (m, 2H), 3.92-3.76 (m, 5H), 3.55 (m, 4H), 2.76-2.58 (m, 2H), 2.40 (m, 4H), 2.08 (m, 3H), 2.00 (s, 3H), 1.96-1.76 (m, 2H). MS m/z 468 (M+1).

### Example 15

### (8S)-N-Methyl-N-({3-[6-(1-pyrrolidinyl)-3-pyridinyl]imidazo[1,2-a]pyridin-2-yl}methyl)-5,6,7,8-tetrahydro-8-quinolinamine

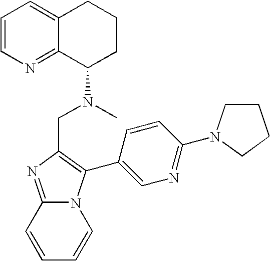

### a) Ethyl 3-[6-(1-pyrrolidinyl)-3-pyridinyl]imidazo[1,2-a]pyridine-2-carboxylate

A solution of ethyl 3-(6-fluoro-3-pyridinyl)imidazo[1,2-a]pyridine-2-carboxylate (195 mg, 0.68 mmol) and pyrrolidine (0.57 mL, 6.83 mmol) in 1,4-dioxane (3 mL) was heated in a 50° bath. After heating for 4 h, the reaction was cooled to room temperature and concentrated to dryness. Flash chromatography (silica gel, 0 to 6% methanol in dichloromethane) afforded 135 mg (59%) of ethyl 3-[6-(1-pyrrolidinyl)-3-pyridinyl]imidazo[1,2-a]pyridine-2-carboxylate as a white solid. ¹H NMR (DMSO-d₆): δ 8.18 (d, 1H), 8.10 (d, 1H), 7.62 (m, 2H), 7.36 (m, 1H), 6.94 (t, 1H), 6.57 (d, 1H), 4.17 (q, 2H), 3.45 (m, 4H), 1.97 (m, 4H), 1.18 (t, 3H). MS m/z 337 (M+1).

### b) {3-[6-(1-Pyrrolidinyl)-3-pyridinyl]imidazo[1,2-a]pyridin-2-yl}methanol

A solution of ethyl 3-[6-(1-pyrrolidinyl)-3-pyridinyl]imidazo[1,2-a]pyridine-2-carboxylate (133 mg, 0.40 mmol) in 15 mL methanol was treated with lithium borohydride (0.8 mL, 2.0 M in THF) and allowed to stir at room temperature. After 1 h, another 0.8 mL portion of lithium borohydride was added, followed by two 1.6 mL portions at 2 h intervals. The reaction was allowed to stir at room temperature overnight, and 4 additional 2 mL portions of lithium borohydride were added over 8 h. After stirring 72 h, methanol (10 mL) and lithium borohydride (2 mL) were added, and these additions were repeated 4 h later. After stirring for 18 h, the reaction was treated with 1 N sodium hydroxide (5 mL) and stirred 15 min, then concentrated. The residue was diluted with water and extracted with ethyl acetate (4×). The combined organic layers were washed with brine, dried over Na₂SO₄ and concentrated. Flash chromatography (silica gel, 0 to 10% methanol in dichloromethane) afforded 51 mg (44%) of {3-[6-(1-pyrrolidinyl)-3-pyridinyl]imidazo[1,2-a]pyridin-2-yl}methanol as an opaque white oil. ¹H NMR (DMSO-d₆): δ 8.23 (d, 1H), 8.16 (d, 1H), 7.67 (m, 1H), 7.52 (m, 1H), 7.22 (m, 1H), 6.84 (t, 1H), 6.59 (d, 1H), 5.02 (t, 1H), 4.42 (d, 2H), 3.43 (m, 4H), 1.95 (m, 4H). MS m/z 295 (M+1).

### c) 3-[6-(1-Pyrrolidinyl)-3-pyridinyl]imidazo[1,2-a]pyridine-2-carbaldehyde

Reaction of {3-[6-(1-pyrrolidinyl)-3-pyridinyl]imidazo[1,2-a]pyridin-2-yl}methanol as described herein for the preparation of 3-[6-(4-methyl-1-piperazinyl)-3-pyridinyl]imidazo[1,2-a]pyridine-2-carbaldehyde afforded 45 mg (92%) of 3-[6-(1-pyrrolidinyl)-3-pyridinyl]imidazo[1,2-a]pyridine-2-carbaldehyde as a yellow solid. ¹H NMR (DMSO-d₆): δ 9.93 (s, 1H), 8.29 (d, 1H), 8.24 (d, 1H), 7.75 (m, 1H), 7.67 (d, 1H), 7.39 (m, 1H), 6.97 (t, 1H), 6.61 (d, 1H), 3.45 (m, 4H), 1.96 (m, 4H).

### d) (8S)-N-Methyl-N-({3-[6-(1-pyrrolidinyl)-3-pyridinyl]imidazo[1,2-a]pyridin-2-yl}methyl)-5,6,7,8-tetrahydro-8-quinolinamine

Reaction of 3-[6-(4-methyl-1-piperazinyl)-3-pyridinyl]imidazo[1,2-a]pyridine-2-carbaldehyde (43 mg, 0.15 mmol) and (8S)-N-methyl-5,6,7,8-tetrahydro-8-quinolinamine (24 mg, 0.15 mmol) as described herein for the preparation of (8S)-N-methyl-N-{[3-(4-pyridinyl)imidazo[1,2-a]pyridin-2-yl]methyl}-5,6,7,8-tetrahydro-8-quinolinamine afforded a quantitative yield of (8S)-N-methyl-N-({3-[6-(1-pyrrolidinyl)-3-pyridinyl]imidazo[1,2-a]pyridin-2-yl}methyl)-5,6,7,8-tetrahydro-8-quinolinamine as a yellow oil. ¹H NMR (DMSO-d₆): δ 8.31 (m, 1H), 8.23 (d, 1H), 8.11 (d, 1H), 7.72 (m, 1H), 7.52 (m, 1H), 7.41 (m, 1H), 7.18 (m, 1H), 7.09 (m, 1H), 6.81 (t, 1H), 6.52 (d, 1H), 3.81 (m, 3H), 3.43 (m, 4H), 2.76-2.57 (m, 2H), 2.08 (s, 3H), 1.96 (m, 4H), 1.90-1.79 (m, 3H), 1.54 (m, 1H). MS m/z 439 (M+1).

### Example 16

### N,N,N′-trimethyl-N′-trimethyl-N′-{5-[2-({methyl[(8S)-5,6,7,8-tetrahydro-8-quinolinyl]amino}methyl)imidazo[1,2-a]pyridin-3-yl]-2-pyridinyl}-1,2-ethanediamine

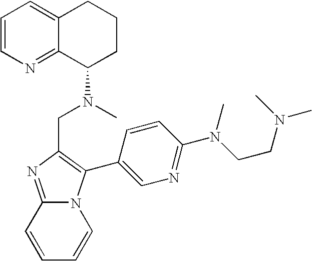

### a) Ethyl 3-{6-[[2-(dimethylamino)ethyl](methyl)amino]-3-pyridinyl}imidazo[1,2-a]pyridine-2-carboxylate

A solution of ethyl 3-(6-fluoro-3-pyridinyl)imidazo[1,2-a]pyridine-2-carboxylate (183 mg, 0.64 mmol) and N,N,N′-trimethyl-1,2-ethanediamine (0.83 mL, 6.41 mmol) in 1,4-dioxane (10 mL) was heated in a 50° bath for 1 h, then the temperature was increased to 60°. After heating for 18 h, the temperature was gradually increased to 90° and maintained at that temperature for 3 h. The temperature was then decreased to 75° and the reaction was heated for 72 h. After that time, the temperature was increased to 90° for 7 h, then the mixture was cooled to room temperature and concentrated to dryness. Flash chromatography (silica gel, 0 to 10% 2 N NH₃/methanol in dichloromethane) afforded 137 mg (58%) of ethyl 3-{6-[[2-(dimethylamino)ethyl](methyl)amino]-3-pyridinyl}imidazo[1,2-a]pyridine-2-carboxylate as a yellow oil. ¹H NMR (DMSO-d₆): δ 8.18 (m, 1H), 8.09 (d, 1H), 7.63 (m, 2H), 7.35 (m, 1H), 6.93 (t, 1H), 6.71 (d, 1H), 4.15 (q, 2H), 3.66 (m, 2H), 3.05 (s, 3H), 2.43 (m, 2H), 2.18 (m, 6H), 1.15 (t, 3H). MS m/z 368 (M+1).

### b) (3-{6-[[2-(Dimethylamino)ethyl](methyl)amino]-3-pyridinyl}imidazo[1,2-a]pyridin-2-yl)methanol

A solution of ethyl 3-{6-[[2-(dimethylamino)ethyl](methyl)amino]-3-pyridinyl}imidazo[1,2-a]pyridine-2-carboxylate (131 mg, 0.36 mmol) in THF (10 mL) was treated with lithium borohydride (1.4 mL, 2.0 M in THF) and allowed to stir at room temperature. After 3 h, the reaction was treated with 1 N sodium hydroxide (5 mL) and stirred 10 min, then concentrated. The residue was diluted with water and extracted with ethyl acetate (2×). The combined organic layers were washed with brine, dried over Na₂SO₄ and concentrated. Flash chromatography (silica gel, 0 to 10% methanol in dichloromethane) afforded 59 mg (51%) of (3-{6-[[2-(dimethylamino)ethyl](methyl)amino]-3-pyridinyl}imidazo[1,2-a]pyridin-2-yl)methanol as a white solid. ¹H NMR (DMSO-d₆): δ 8.28 (d, 1H), 8.19 (d, 1H), 7.73 (m, 1H), 7.53 (m, 1H), 7.22 (m, 1H), 6.83 (m, 2H), 5.04 (t, 1H), 4.43 (d, 2H), 3.94 (m, 2H), 3.06 (s, 3H), 2.89 (m, 2H), 2.58 (s, 6H). MS m/z 326 (M+1).

### c) 3-{6-[[2-(Dimethylamino)ethyl](methyl)amino]-3-pyridinyl}imidazo[1,2-a]pyridine-2-carbaldehyde

Reaction of (3-{6-[[2-(dimethylamino)ethyl](methyl)amino]-3-pyridinyl}imidazo[1,2-a]pyridin-2-yl)methanol (54 mg, 0.17 mmol) as described herein for the preparation of 3-[6-(4-methyl-1-piperazinyl)-3-pyridinyl]imidazo[1,2-a]pyridine-2-carbaldehyde afforded 47 mg (87%) of 3-{6-[[2-(dimethylamino)ethyl]methyl)amino]-3-pyridinyl}imidazo[1,2-a]pyridine-2-carbaldehyde as a yellow foam. ¹H NMR (DMSO-d₆): δ 9.94 (s, 1H), 8.34 (d, 1H), 8.26 (d, 1H), 7.81 (m, 1H), 7.68 (m, 1H), 7.40 (m, 1H), 6.98 (t, 1H), 6.82 (d, 1H), 3.96 (m, 2H), 3.09 (s, 3H), 2.90 (m, 2H), 2.58 (s, 6H).

d) N,N,N′-Trimethyl-N′-{5-[2-({methyl[(8S)-5,6,7,8-tetrahydro-8-quinolinyl]amino}methyl)imidazo[1,2-a]pyridin-3-yl}-2-pyridinyl}-1,2-ethanediamine

Reaction of 3-{6-[[2-(dimethylamino)ethyl](methyl)amino]-3-pyridinyl}imidazo[1,2-a]pyridine-2-carbaldehyde (44 mg, 0.14 mmol) and (8S)-N-methyl-5,6,7,8-tetrahydro-8-quinolinamine (22 mg, 0.14 mmol) as described herein for the preparation of (8S)-N-methyl-N-{[3-(4-pyridinyl)imidazo[1,2-a]pyridin-2-yl]methyl}-5,6,7,8-tetrahydro-8-quinolinamine afforded 42 mg (66%) of N,N,N′-trimethyl-N′-{5-[2-({methyl[(8S)-5,6,7,8-tetrahydro-8-quinolinyl]amino}methyl)imidazo[1,2-a]pyridin-3-yl]-2-pyridinyl}-1,2-ethanediamine as a pale yellow oil. ¹H NMR (DMSO-d₆): δ 8.30 (m, 2H), 8.13 (d, 1H), 7.77 (m, 1H), 7.52 (d, 1H), 7.41 (d, 1H), 7.19 (m, 1H), 7.09 (m, 1H), 6.82 (t, 1H), 6.73 (d, 1H), 4.01-3.76 (m, 5H), 3.06 (s, 3H), 2.90 (m, 2H), 2.75-2.58 (m, 8H), 2.10 (s, 3H), 1.92-1.52 (m, 4H). MS m/z 470 (M+1).

### Example 17

### N,N,N′-Trimethyl-N′-{[2-({methyl[(8S)-5,6,7,8-tetrahydro-8-quinolinyl]amino}methyl)imidazo[1,2-a]pyridin-3-yl]methyl}-1,2-ethanediamine

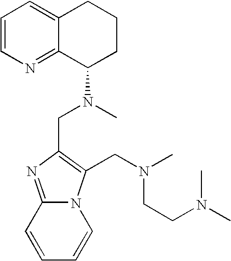

Reaction of (8S)-N-(imidazo[1,2-a]pyridin-2-ylmethyl)-N-methyl-5,6,7,8-tetrahydro-8-quinolinamine (66 mg, 0.23 mmol) and N,N,N-trimethyl-1,2-ethanediamine (0.29 mL, 2.23 mmol) as described herein for the preparation of (8S)-N-methyl-N-{[3-(4-morpholinylmethyl)imidazo[1,2-a]pyridin-2-yl]methyl}-5,6,7,8-tetrahydro-8-quinolinamine afforded 40 mg (43%) of N,N,N′-trimethyl-N-{[2-({methyl[(8S)-5,6,7,8-tetrahydro-8-quinolinyl]amino}methyl)imidazo[1,2-a]pyridin-3-yl]methyl}-1,2-ethanediamine as a pale gold oil. ¹H NMR (DMSO-d₆): δ 8.44 (d, 1H), 8.40 (m, 1H), 7.45 (m, 2H), 7.15 (m, 2H), 6.82 (t, 1H), 3.93-3.70 (m, 5H), 2.83-2.64 (m, 2H), 2.37-2.27 (m, 4H), 2.11 (s, 3H), 2.04 (s, 6H), 2.02-1.82 (m, 6H), 1.61 (m, 1H). MS m/z 407 (M+1).

### Example 18

### (8S)-N-[(3-{6-[3-(Dimethylamino)-1-pyrrolidinyl]-3-pyridinyl}imidazo[1,2-a]pyridin-2-yl)methyl]-N-methyl-5,6,7,8-tetrahydro-8-quinolinamine

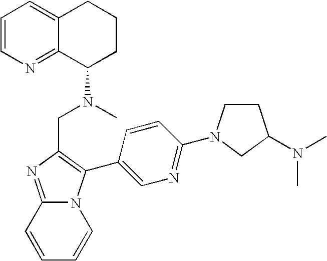

### a) Ethyl 3-{6-[3-(dimethylamino)-1-pyrrolidinyl]-3-pyridinyl}imidazo[1,2-a]pyridine-2-carboxylate

A solution of ethyl 3-(6-fluoro-3-pyridinyl)imidazo[1,2-a]pyridine-2-carboxylate (198 mg, 0.69 mmol) and N,N-dimethyl-3-pyrrolidinamine (0.88 mL, 6.94 mmol) in 1,4-dioxane (4 mL) was heated in a 50° bath for 2 h, then cooled to room temperature overnight. Reheated to 50° the next day for 1 h, then cooled to room temperature and concentrated to dryness. Flash chromatography (silica gel, 0 to 10% methanol in dichloromethane) afforded 173 mg (66%) of ethyl 3-{6-[3-(dimethylamino)-1-pyrrolidinyl]-3-pyridinyl}imidazo[1,2-a]pyridine-2-carboxylate as a tan oil which partially solidified upon standing. ¹H NMR (DMSO-d₆): δ 8.17 (d, 1H), 8.08 (d, 1H), 7.63 (m, 2H), 7.34 (m, 1H), 6.93 (t, 1H), 6.58 (d, 1H), 4.16 (q, 2H), 3.75-3.61 (m, 2H), 3.36 (m, 1H), 3.16 (m, 1H), 2.20 (m, 7H), 1.81 (m, 1H), 1.17 (t, 3H). MS m/z 380 (M+1).

### b) (3-{6-[3-(dimethylamino)-1-pyrrolidinyl]-3-pyridinyl}imidazo[1,2-a]pyridin-2-yl)methanol

A solution of ethyl 3-{6-[3-(dimethylamino)-1-pyrrolidinyl]-3-pyridinyl}imidazo[1,2-a]pyridine-2-carboxylate (164 mg, 0.43 mmol) in THF (10 mL) was treated with lithium borohydride (1.7 mL, 2.0 M in THF) and allowed to stir at room temperature. After 4 h, the reaction was treated with 1 N sodium hydroxide (5 mL) and stirred 10 min, then concentrated. The residue was diluted with water and extracted with ethyl acetate (2×). The combined organic layers were washed with brine, dried over Na₂SO₄ and concentrated. Flash chromatography (silica gel, 0 to 10% methanol in dichloromethane) afforded 43 mg (29%) of (3-{6-[3-(dimethylamino)-1-pyrrolidinyl]-3-pyridinyl}imidazo[1,2-a]pyridin-2-yl)methanol as a white solid. ¹H NMR (DMSO-d₆): δ 8.27 (d, 1H), 8.17 (d, 1H), 7.72 (m, 1H), 7.53 (d, 1H), 7.23 (m, 1H), 6.85 (t, 1H), 6.65 (d, 1H), 5.03 (t, 1H), 4.43 (d, 2H), 3.76-3.62 (m, 4H), 3.34 (m, 1H), 2.55 (d, 6H), 2.25 (m, 2H). MS m/z 338 (M+1).

### c) 3-{6-[3-(Dimethylamino)-1-pyrrolidinyl]-3-pyridinyl}imidazo[1,2-a]pyridine-2-carbaldehyde

Reaction of (3-{6-[3-(dimethylamino)-1-pyrrolidinyl]-3-pyridinyl}imidazo[1,2-a]pyridin-2-yl)methanol (41 mg, 0.12 mmol) as described herein for the preparation of 3-[6-(4-methyl-1-piperazinyl)-3-pyridinyl]imidazo[1,2-a]pyridine-2-carbaldehyde afforded 24 mg (59%) of 3-{6-[3-(dimethylamino)-1-pyrrolidinyl]-3-pyridinyl}imidazo[1,2-a]pyridine-2-carbaldehyde as a yellow oil. ¹H NMR (DMSO-d₆): δ 9.93 (s, 1H), 8.33 (d, 1H), 8.24 (d, 1H), 7.80 (m, 1H), 7.68 (m, 1H), 7.40 (m, 1H), 6.98 (t, 1H), 6.67 (d, 1H), 3.71 (m, 4H), 3.38 (m, 1H), 2.56 (d, 6H), 2.28 (m, 2H).

### d) (8S)-N-[(3-{6-[3-(Dimethylamino)-1-pyrrolidinyl]-3-pyridinyl}imidazo[1,2-a]pyridin-2-yl)methyl]-N-methyl-5,6,7,8-tetrahydro-8-quinolinamine

Reaction of 3-{6-[3-(dimethylamino)-1-pyrrolidinyl]-3-pyridinyl}imidazo[1,2-a]pyridine-2-carbaldehyde (24 mg, 0.072 mmol) and (8S)-N-methyl-5,6,7,8-tetrahydro-8-quinolinamine (12 mg, 0.072 mmol) as described herein for the preparation of (8S)-N-methyl-N-{[3-(4-pyridinyl)imidazo[1,2-a]pyridin-2-yl]methyl}-5,6,7,8-tetrahydro-8-quinolinamine afforded 16 mg (47%) of (8S)-N-[(3-{6-[3-(dimethylamino)-1-pyrrolidinyl]-3-pyridinyl}imidazo[1,2-a]pyridin-2-yl)methyl]-N-methyl-5,6,7,8-tetrahydro-8-quinolinamine as a colorless oil. ¹H NMR (CD₃OD): δ 8.19 (d, 1H), 8.06 (m, 2H), 7.62 (m, 1H), 7.52 (m, 1H), 7.42 (m, 1H), 7.28 (m, 1H), 7.08 (m, 1H), 6.86 (t, 1H), 6.61 (d, 1H), 3.78 (m, 5H), 3.48 (m, 1H), 2.97 (m, 1H), 2.79 (m, 1H), 2.65 (m, 1H), 2.37 (m, 8H), 2.27 (s, 3H), 2.06-1.83 (m, 4H), 1.61 (m, 1H). MS m/z 482 (M+1).

### Example 19

### (8S)-N-{[3-({Bis[2-(methyloxy)ethyl]amino}methyl)imidazo[1,2-a]pyridin-2-yl]methyl}-N-methyl-5,6,7,8-tetrahydro-8-quinolinamine

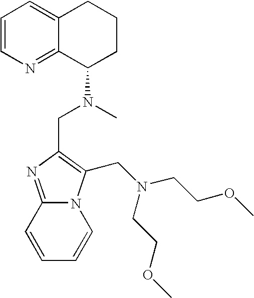

Reaction of (8S)-N-(imidazo[1,2-a]pyridin-2-ylmethyl)-N-methyl-5,6,7,8-tetrahydro-8-quinolinamine (63 mg, 0.22 mmol) and bis[2-(methyloxy)ethyl]amine (0.32 mL, 2.15 mmol) as described herein for the preparation of (8S)-N-methyl-N-{[3-(4-morpholinylmethyl)imidazo[1,2-a]pyridin-2-yl]methyl}-5,6,7,8-tetrahydro-8-quinolinamine afforded 47 mg (50%) of (8S)-N-{[3-({bis[2-(methyloxy)ethyl]amino}methyl)imidazo[1,2-a]pyridin-2-yl]methyl}-N-methyl-5,6,7,8-tetrahydro-8-quinolinamine as a gold oil. ¹H NMR (DMSO-d₆): δ 8.41 (m, 2H), 7.46 (m, 2H), 7.16 (m, 2H), 6.83 (t, 1H), 3.97-3.76 (m, 5H), 3.28 (m, 4H), 3.11 (s, 6H), 2.81-2.63 (m, 4H), 2.11 (s, 3H), 2.00-1.85 (m, 3H), 1.61 (m, 1H). MS m/z 438 (M+1).

### Example 20

### (8S)-N-({3-[(Diethylamino)methyl]imidazo[1,2-a]pyridin-2-yl}methyl)-N-methyl-5,6,7,8-tetrahydro-8-quinolinamine

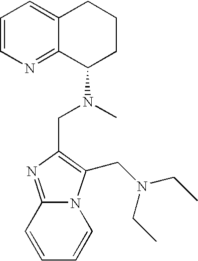

Reaction of (8S)-N-(imidazo[1,2-a]pyridin-2-ylmethyl)-N-methyl-5,6,7,8-tetrahydro-8-quinolinamine (87 mg, 0.30 mmol) and diethylamine (0.31 mL, 2.98 mmol) as described herein for the preparation of (8S)-N-methyl-N-{[3-(4-morpholinylmethyl)imidazo[1,2-a]pyridin-2-yl]methyl}-5,6,7,8-tetrahydro-8-quinolinamine afforded 57 mg (51%) of (8S)-N-({3-[(diethylamino)methyl]imidazo[1,2-a]pyridin-2-yl}methyl)-N-methyl-5,6,7,8-tetrahydro-8-quinolinamine as a gold oil. ¹H NMR (DMSO-d₆): δ 8.39 (m, 1H), 8.32 (d, 1H), 7.46 (m, 2H), 7.15 (m, 2H), 6.84 (t, 1H), 3.93-3.76 (m, 5H), 2.82-2.63 (m, 2H), 2.34 (q, 4H), 2.11 (s, 3H), 2.00-1.82 (m, 3H), 1.60 (m, 1H), 0.88 (t, 6H). MS m/z 378 (M+1).

### Example 21

### (8S)-N-Methyl-N-{[3-({methyl[2-(methyloxy)ethyl]amino}methyl)imidazo[1,2-a]pyridin-2-yl]methyl}-5,6,7,8-tetrahydro-8-quinolinamine

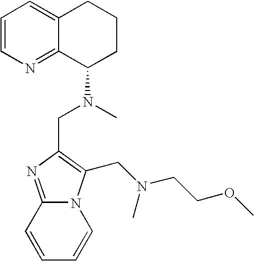

Reaction of (8S)-N-(imidazo[1,2-a]pyridin-2-ylmethyl)-N-methyl-5,6,7,8-tetrahydro-8-quinolinamine (57 mg, 0.19 mmol) and methyl[2-(methyloxy)ethyl]amine (0.21 mL, 1.95 mmol) as described herein for the preparation of (8S)-N-methyl-N-{[3-(4-morpholinylmethyl)imidazo[1,2-a]pyridin-2-yl]methyl}-5,6,7,8-tetrahydro-8-quinolinamine afforded 52 mg (67%) of (8S)-N-methyl-N-{[3-({methyl[2-(methyloxy)ethyl]amino}methyl)imidazo[1,2-a]pyridin-2-yl]methyl}-5,6,7,8-tetrahydro-8-quinolinamine as a yellow oil. ¹H NMR (DMSO-d₆): δ 8.40 (d, 1H), 8.34 (d, 1H), 7.46 (m, 2H), 7.16 (m, 2H), 6.83 (t, 1H), 3.93-3.74 (m, 5H), 3.37 (t, 2H), 3.16 (s, 3H), 2.83-2.63 (m, 2H), 2.45 (m, 2H), 2.11 (s, 3H), 2.02-1.85 (m, 6H), 1.61 (m, 1H). MS m/z 394 (M+1).

### Example 22

### (8S)-N-Methyl-N-[(3-{[methyl(1-methylethyl)amino]methyl}imidazo[1,2-a]pyridin-2-yl)methyl]-5,6,7,8-tetrahydro-8-quinolinamine

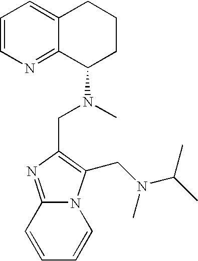

Reaction of (8S)-N-(imidazo[1,2-a]pyridin-2-ylmethyl)-N-methyl-5,6,7,8-tetrahydro-8-quinolinamine (84 mg, 0.29 mmol) and methyl(1-methylethyl)amine (0.30 mL, 2.87 mmol) as described herein for the preparation of (8S)-N-methyl-N-{[3-(4-morpholinylmethyl)imidazo[1,2-a]pyridin-2-yl]methyl}-5,6,7,8-tetrahydro-8-quinolinamine afforded 73 mg (68%) of (8S)-N-methyl-N-[(3-{[methyl(1-methylethyl)amino]methyl}imidazo[1,2-a]pyridin-2-yl)methyl]-5,6,7,8-tetrahydro-8-quinolinamine as a yellow oil. ¹H NMR (DMSO-d₆): δ 8.39 (d, 1H), 8.29 (d, 1H), 7.45 (m, 2H), 7.15 (m, 2H), 6.83 (t, 1H), 3.92-3.76 (m, 5H), 2.82-2.63 (m, 3H), 2.11 (s, 3H), 2.01-1.88 (m, 6H), 1.60 (m, 1H), 0.97 (m, 6H). MS m/z 378 (M+1).

### Example 23

### (8S)-N-Methyl-N-[(3-{[methyl(1-methyl-3-pyrrolidinyl)amino]methyl}imidazo[1,2-a]pyridin-2-yl)methyl]-5,6,7,8-tetrahydro-8-quinolinamine

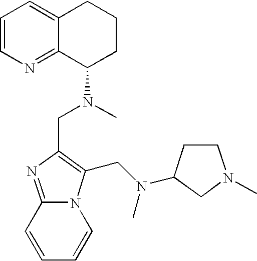

Reaction of (8S)-N-(imidazo[1,2-a]pyridin-2-ylmethyl)-N-methyl-5,6,7,8-tetrahydro-8-quinolinamine (64 mg, 0.22 mmol) and N,1-dimethyl-3-pyrrolidinamine (0.25 mL, 2.19 mmol) as described herein for the preparation of (8S)-N-methyl-N-{[3-(4-morpholinylmethyl)imidazo[1,2-a]pyridin-2-yl]methyl}-5,6,7,8-tetrahydro-8-quinolinamine afforded 32 mg (35%) of (8S)-N-methyl-N-[(3-{[methyl(1-methyl-3-pyrrolidinyl)amino]methyl}imidazo[1,2-a]pyridin-2-yl)methyl]-5,6,7,8-tetrahydro-8-quinolinamine as a pale yellow oil. ¹H NMR (DMSO-d₆): δ 8.40 (d, 1H), 8.28 (d, 1H), 7.46 (m, 2H), 7.15 (m, 2H), 6.84 (t, 1H), 3.92-3.67 (m, 5H), 3.03 (m, 1H), 2.83-2.64 (m, 2H), 2.53-2.37 (m, 5H), 2.19 (s, 3H), 2.11 (s, 3H), 1.97 (m, 2H), 1.83 (m, 4H), 1.76-1.58 (m, 2H). MS m/z 419 (M+1).

### Example 24

### (8S)-N-Methyl-N-[(3-{[methyl(1-methyl-4-piperidinyl)amino]methyl}imidazo[1,2-a]pyridin-2-yl)methyl]-5,6,7,8-tetrahydro-8-quinolinamine

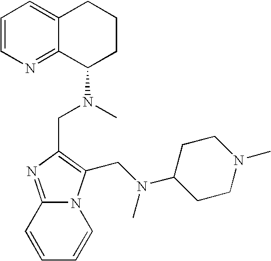

Reaction of (8S)-N-(imidazo[1,2-a]pyridin-2-ylmethyl)-N-methyl-5,6,7,8-tetrahydro-8-quinolinamine (54 mg, 0.18 mmol) and N,1-dimethyl-4-piperidinamine (0.27 mL, 1.85 mmol) as described herein for the preparation of (8S)-N-methyl-N-{[3-(4-morpholinylmethyl)imidazo[1,2-a]pyridin-2-yl]methyl}-5,6,7,8-tetrahydro-8-quinolinamine afforded 45 mg (56%) of (8S)-N-methyl-N-[(3-{[methyl(1-methyl-4-piperidinyl)amino]methyl}imidazo[1,2-a]pyridin-2-yl)methyl]-5,6,7,8-tetrahydro-8-quinolinamine as a yellow oil. ¹H NMR (DMSO-d₆): δ 8.38 (m, 1H), 8.27 (d, 1H), 7.46 (m, 2H), 7.15 (m, 2H), 6.82 (t, 1H), 3.91-3.73 (m, 5H), 2.81-2.63 (m, 4H), 2.27 (m, 1H), 2.11 (s, 3H), 2.09 (s, 3H), 2.04-1.92 (m, 5H), 1.86-1.71 (m, 3H), 1.63-1.47 (m, 5H). MS m/z 433 (M+1).

### Example 25

### (Methyl{[2-({methyl[(8S)-5,6,7,8-tetrahydro-8-quinolinyl]amino}methyl)imidazo[1,2-a]pyridin-3-yl]methyl}amino)acetonitrile

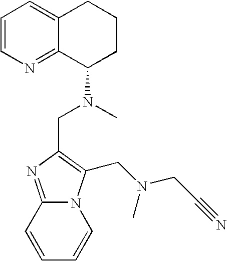

Reaction of (8S)-N-(imidazo[1,2-a]pyridin-2-ylmethyl)-N-methyl-5,6,7,8-tetrahydro-8-quinolinamine (256 mg, 0.88 mmol) and (methylamino)acetonitrile (0.67 mL, 8.76 mmol) as described herein for the preparation of (8S)-N-methyl-N-{[3-(4-morpholinylmethyl)imidazo[1,2-a]pyridin-2-yl]methyl}-5,6,7,8-tetrahydro-8-quinolinamine afforded 262 mg (80%) of (methyl{[2-({methyl[(8S)-5,6,7,8-tetrahydro-8-quinolinyl]amino}methyl)imidazo[1,2-a]pyridin-3-yl]methyl}amino)acetonitrile as a brown oil. ¹H NMR (DMSO-d₆): δ 8.40 (d, 1H), 8.27 (d, 1H), 7.46 (m, 2H), 7.17 (m, 2H), 6.87 (t, 1H), 3.99-3.87 (m, 5H), 3.74 (m, 2H), 2.82-2.61 (m, 2H), 2.19 (s, 3H), 2.11 (s, 3H), 1.95 (m, 3H), 1.60 (m, 1H). MS m/z 375 (M+1).

### Example 26

### (8S)-N-Methyl-N-[(3-{[methyl(2-methylpropyl)amino]methyl}imidazo[1,2-a]pyridin-2-yl)methyl]-5,6,7,8-tetrahydro-8-quinolinamine

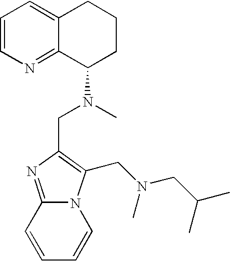

Reaction of (8S)-N-(imidazo[1,2-a]pyridin-2-ylmethyl)-N-methyl-5,6,7,8-tetrahydro-8-quinolinamine (66 mg, 0.23 mmol) and methyl(2-methylpropyl)amine (0.27 mL, 2.26 mmol) as described herein for the preparation of (8S)-N-methyl-N-{[3-(4-morpholinylmethyl)imidazo[1,2-a]pyridin-2-yl]methyl}-5,6,7,8-tetrahydro-8-quinolinamine afforded 62 mg (70%) of (8S)-N-methyl-N-[(3-{[methyl(2-methylpropyl)amino]methyl}imidazo[1,2-a]pyridin-2-yl)methyl]-5,6,7,8-tetrahydro-8-quinolinamine as a gold oil. ¹H NMR (DMSO-d₆): δ 8.40 (d, 1H), 8.29 (d, 1H), 7.46 (m, 2H), 7.16 (m, 2H), 6.84 (t, 1H), 3.94-3.67 (m, 5H), 2.84-2.64 (m, 2H), 2.12 (s, 3H), 1.99-1.83 (m, 8H), 1.63 (m, 2H), 0.67 (m, 6H). MS m/z 392 (M+1).

### Example 27

### 3-(Methyl{[2-({methyl[(8S)-5,6,7,8-tetrahydro-8-quinolinyl]amino}methyl)imidazo[1,2-a]pyridin-3-yl]methyl}amino)propanenitrile

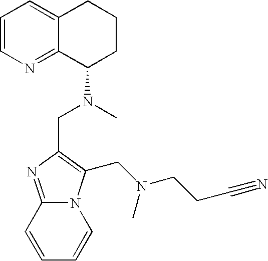

Reaction of (8S)-N-(imidazo[1,2-a]pyridin-2-ylmethyl)-N-methyl-5,6,7,8-tetrahydro-8-quinolinamine (233 mg, 0.80 mmol) and 3-(methylamino)propanenitrile (0.75 mL, 7.97 mmol) as described herein for the preparation of (8S)-N-methyl-N-{[3-(4-morpholinylmethyl)imidazo[1,2-a]pyridin-2-yl]methyl}-5,6,7,8-tetrahydro-8-quinolinamine afforded 155 mg (50%) of 3-(methyl{[2-({methyl[(8S)-5,6,7,8-tetrahydro-8-quinolinyl]amino}methyl)imidazo[1,2-a]pyridin-3-yl]methyl}amino)propanenitrile as a pale yellow oil after a second purification by reverse-phase HPLC followed by free basing with dichloromethane and aqueous 10% sodium carbonate. ¹H NMR (DMSO-d₆): δ 8.41 (d, 1H), 8.36 (d, 1H), 7.46 (m, 2H), 7.16 (m, 2H), 6.81 (t, 1H), 3.94-3.76 (m, 5H), 2.82-2.56 (m, 6H), 2.11 (s, 3H), 2.05 (s, 3H), 1.95 (m, 3H), 1.61 (m, 1H). MS m/z 389 (M+1).

### Example 28

### (8S)-N-({3-[2-(Dimethylamino)-4-pyridinyl]imidazo[1,2-a]pyridin-2-yl}methyl)-N-methyl-5,6,7,8-tetrahydro-8-quinolinamine

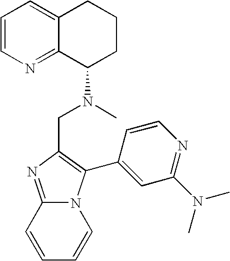

### a) Ethyl 3-(2-fluoro-4-pyridinyl)imidazo[1,2-a]pyridine-2-carboxylate

Reaction of ethyl 3-iodoimidazo[1,2-a]pyridine-2-carboxylate (2.06 g, 6.52 mmol) and (2-fluoro-4-pyridinyl)boronic acid (1.01 g, 7.38 mmol) as described herein for the preparation of ethyl 3-(6-fluoro-3-pyridinyl)imidazo[1,2-a]pyridine-2-carboxylate afforded 0.79 g (42%) of ethyl 3-(2-fluoro-4-pyridinyl)imidazo[1,2-a]pyridine-2-carboxylate as a pale yellow solid. ¹H NMR (DMSO-d₆): δ 8.39 (d, 1H), 8.25 (d, 1H), 7.72 (m, 1H), 7.61-7.50 (m, 2H), 7.44 (m, 1H), 7.01 (t, 1H), 4.18 (q, 2H), 1.14 (t, 3H). MS m/z 286 (M+1).

### b) Ethyl 3-[2-(dimethylamino)-4-pyridinyl]imidazo[1,2-a]pyridine-2-carboxylate

A mixture of ethyl 3-(2-fluoro-4-pyridinyl)imidazo[1,2-a]pyridine-2-carboxylate (270 mg, 0.95 mmol) and dimethylamine (5 mL, 2.0 M in THF) was heated in a sealed tube in an 80° bath. After heating for 24 h, the reaction was cooled to room temperature and concentrated to dryness. Flash chromatography (silica gel, 0 to 5% methanol in dichloromethane) afforded 256 mg (87%) of ethyl 3-[2-(dimethylamino)-4-pyridinyl]imidazo[1,2-a]pyridine-2-carboxylate as a light tan oil which partially solidified upon standing. ¹H NMR (DMSO-d₆): δ 8.21 (d, 1H), 8.15 (d, 1H), 7.67 (m, 1H), 7.39 (m, 1H), 6.96 (t, 1H), 6.78 (s, 1H), 6.66 (d, 1H), 4.16 (q, 2H), 3.03 (s, 6H), 1.14 (t, 3H). MS m/z 311 (M+1).

### c) {3-[2-(Dimethylamino)-4-pyridinyl]imidazo[1,2-a]pyridin-2-yl}methanol

A solution of ethyl 3-[2-(dimethylamino)-4-pyridinyl]imidazo[1,2-a]pyridine-2-carboxylate (248 mg, 0.80 mmol) in 15 mL anhydrous isopropanol was treated with lithium borohydride (2.4 mL, 2.0 M in THF) and allowed to stir at room temperature. After 3 h, the reaction was treated with 1 N sodium hydroxide (5 mL) and stirred 15 min, then concentrated. The residue was diluted with water and extracted with ethyl acetate (3×). The combined organic layers were washed with brine, dried over Na₂SO₄ and concentrated. Flash chromatography (silica gel, 0 to 10% methanol in dichloromethane) afforded 96 mg (45%) of {3-[2-(dimethylamino)-4-pyridinyl]imidazo[1,2-a]pyridin-2-yl}methanol as a white solid. ¹H NMR (DMSO-d₆): δ 8.46 (d, 1H), 8.20 (d, 1H), 7.58 (m, 1H), 7.29 (m, 1H), 6.91 (t, 1H), 6.84 (s, 1H), 6.80 (d, 1H), 5.18 (t, 1H), 4.51 (d, 2H), 3.05 (s, 6H).

### d) 3-[2-(Dimethylamino)-4-pyridinyl]imidazo[1,2-a]pyridine-2-carbaldehyde

Reaction of {3-[2-(dimethylamino)-4-pyridinyl]imidazo[1,2-a]pyridin-2-yl}methanol (96 mg, 0.36 mmol) as described herein for the preparation of 3-[6-(4-methyl-1-piperazinyl)-3-pyridinyl]imidazo[1,2-a]pyridine-2-carbaldehyde afforded 77 mg (81%) of 3-[2-(dimethylamino)-4-pyridinyl]imidazo[1,2-a]pyridine-2-carbaldehyde as a pale yellow solid. ¹H NMR (DMSO-d₆): δ 10.00 (s, 1H), 8.36 (d, 1H), 8.25 (d, 1H), 7.72 (m, 1H), 7.44 (m, 1H), 7.02 (t, 1H), 6.90 (s, 1H), 6.80 (d, 1H), 3.05 (s, 6H).

### e) (8S)-N-({3-[2-(Dimethylamino)-4-pyridinyl]imidazo[1,2-a]pyridin-2-yl}methyl)-N-methyl-5,6,7,8-tetrahydro-8-quinolinamine

Reaction of 3-[2-(dimethylamino)-4-pyridinyl]imidazo[1,2-a]pyridine-2-carbaldehyde (77 mg, 0.29 mmol) and (8S)-N-methyl-5,6,7,8-tetrahydro-8-quinolinamine (47 mg, 0.29 mmol) as described herein for the preparation of (8S)-N-methyl-N-{[3-(4-pyridinyl)imidazo[1,2-a]pyridin-2-yl]methyl}-5,6,7,8-tetrahydro-8-quinolinamine afforded 104 mg (87%) of (8S)-N-({3-[2-(dimethylamino)-4-pyridinyl]imidazo[1,2-a]pyridin-2-yl}methyl)-N-methyl-5,6,7,8-tetrahydro-8-quinolinamine as a peach-colored foam. ¹H NMR (DMSO-d₆): δ 8.41 (d, 1H), 8.30 (d, 1H), 8.15 (d, 1H), 7.58 (m, 1H), 7.40 (m, 1H), 7.25 (m, 1H), 7.09 (m, 2H), 6.89 (t, 1H), 6.83 (d, 1H), 3.88 (m, 3H), 3.04 (s, 6H), 2.74-2.58 (m, 2H), 2.11 (s, 3H), 1.87 (m, 3H), 1.52 (m, 1H). MS m/z 413 (M+1).

### Example 29

### (8S)-N-Methyl-N-({3-[2-(4-morpholinyl)-4-pyridinyl]imidazo[1,2-a]pyridin-2-yl}methyl)-5,6,7,8-tetrahydro-8-quinolinamine

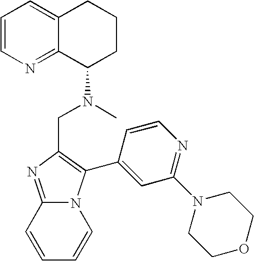

### a) Ethyl 3-[2-(4-morpholinyl)-4-pyridinyl]imidazo[1,2-a]pyridine-2-carboxylate

A solution of ethyl 3-(2-fluoro-4-pyridinyl)imidazo[1,2-a]pyridine-2-carboxylate (246 mg, 0.86 mmol) and morpholine (0.75 mL, 8.62 mmol) in 1,4-dioxane (5 mL) was heated in a 70° bath for 2 h, then the temperature was increased to 80° for 24 h. The reaction was cooled to room temperature and concentrated to dryness. Flash chromatography (silica gel, 0 to 5% methanol in dichloromethane) afforded 226 mg (74%) of ethyl 3-[2-(4-morpholinyl)-4-pyridinyl]imidazo[1,2-a]pyridine-2-carboxylate as a colorless oil which mostly solidified upon standing. ¹H NMR (DMSO-d₆): δ 8.26 (m, 1H), 8.14 (d, 1H), 7.68 (d, 1H), 7.39 (m, 1H), 6.98 (m, 2H), 6.80 (d, 1H), 4.16 (q, 2H), 3.67 (m, 4H), 3.47 (m, 4H), 1.14 (t, 3H). MS m/z 353 (M+1).

### b) {3-[2-(4-Morpholinyl)-4-pyridinyl]imidazo[1,2-a]pyridin-2-yl}methanol

Reaction of ethyl 3-[2-(4-morpholinyl)-4-pyridinyl]imidazo[1,2-a]pyridine-2-carboxylate (222 mg, 0.63 mmol) as described herein for the preparation of {3-[2-(dimethylamino)-4-pyridinyl]imidazo[1,2-a]pyridin-2-yl}methanol afforded 62 mg (32%) of {3-[2-(4-morpholinyl)-4-pyridinyl]imidazo[1,2-a]pyridin-2-yl}methanol as a white solid. ¹H NMR (DMSO-d₆): δ 8.45 (d, 1H), 8.25 (d, 1H), 7.59 (m, 1H), 7.30 (m, 1H), 7.04 (s, 1H), 6.92 (m, 2H), 5.20 (t, 1H), 4.50 (d, 2H), 3.69 (m, 4H), 3.47 (m, 4H). MS m/z 311 (M+1).

### c) 3-[2-(4-Morpholinyl)-4-pyridinyl]imidazo[1,2-a]pyridine-2-carbaldehyde

Reaction of {3-[2-(4-morpholinyl)-4-pyridinyl]imidazo[1,2-a]pyridin-2-yl}methanol (60 mg, 0.19 mmol) as described herein for the preparation of 3-[6-(4-methyl-1-piperazinyl)-3-pyridinyl]imidazo[1,2-a]pyridine-2-carbaldehyde afforded 56 mg (93%) of 3-[2-(4-morpholinyl)-4-pyridinyl]imidazo[1,2-a]pyridine-2-carbaldehyde as a pale yellow solid. ¹H NMR (DMSO-d₆): δ 9.98 (s, 1H), 8.35 (d, 1H), 8.30 (d, 1H), 7.72 (m, 1H), 7.45 (m, 1H), 7.12 (s, 1H), 7.03 (t, 1H), 6.93 (d, 1H), 3.68 (m, 4H), 3.50 (m, 4H). MS m/z 309 (M+1).

### d) (8S)-N-Methyl-N-({3-[2-(4-morpholinyl)-4-pyridinyl]imidazo[1,2-a]pyridin-2-yl}methyl)-5,6,7,8-tetrahydro-8-quinolinamine

Reaction of 3-[2-(4-morpholinyl)-4-pyridinyl]imidazo[1,2-a]pyridine-2-carbaldehyde (55 mg, 0.18 mmol) and (8S)-N-methyl-5,6,7,8-tetrahydro-8-quinolinamine (29 mg, 0.18 mmol) as described herein for the preparation of (8S)-N-methyl-N-{[3-(4-pyridinyl)imidazo[1,2-a]pyridin-2-yl]methyl}-5,6,7,8-tetrahydro-8-quinolinamine afforded 77 mg (95%) of (8S)-N-methyl-N-({3-[2-(4-morpholinyl)-4-pyridinyl]imidazo[1,2-a]pyridin-2-yl}methyl)-5,6,7,8-tetrahydro-8-quinolinamine as a peach-colored foam. ¹H NMR (DMSO-d₆): δ 8.43 (d, 1H), 8.30 (d, 1H), 8.20 (d, 1H), 7.59 (m, 1H), 7.51 (s, 1H), 7.41 (m, 1H), 7.27 (m, 1H), 7.09 (m, 1H), 6.98 (d, 1H), 6.91 (m, 1H), 3.90-3.80 (m, 3H), 3.63 (m, 4H), 3.51 (m, 4H), 2.75-2.59 (m, 2H), 2.08 (s, 3H), 1.94-1.82 (m, 3H), 1.55 (m, 1H). MS m/z 455 (M+1).

### Example 30

### N,N-dimethyl-N′-[(2-{[methyl(5,6,7,8-tetrahydro-8-quinolinyl)amino]methyl}imidazo[1,2-a]pyridin-3-yl)methyl]-1,2-ethanediamine

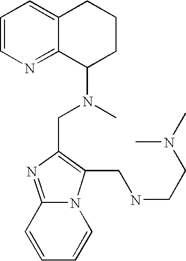

### a) 2-[(3-bromoimidazo[1,2-a]pyridin-2-yl)methyl]-1H-isoindole-1,3(2H)-dione

Dissolved 2-(imidazo[1,2-a]pyridin-2-ylmethyl)-1H-isoindole-1,3(2H)-dione (2.0 g, 7.22 mmol; prepared in a similar fashion as described in WO00/26203) in glacial acetic acid (60 mL) and added bromine (9.0 mL, 1M solution in trimethylphosphate, 14.4 mmol). Let react at room temperature for one hour. Filtered off the solid and stirred vigorously in water while adding ammonium hydroxide until solution was basic. Filtered the solid affording 1.13 (44% yield) of 2-[(3-bromoimidazo[1,2-a]pyridin-2-yl)methyl]-1H-isoindole-1,3(2H)-dione. ¹H NMR (400 MHz, DMSO-D6) δ 4.91 (s, 2H), 7.14 (t, 1H), 7.59 (d, 1H), 7.83-7.92 (m, 4H), 8.36 (d, 1H).

### b) [(3-bromoimidazo[1,2-a]pyridin-2-yl)methyl]amine

Dissolved 2-[(3-bromoimidazo[1,2-a]pyridin-2-yl)methyl]-1H-isoindole-1,3(2H)-dione (1.31 g, 3.69 mmol) in anhydrous methanol (9 mL) and added hydrazine (0.23 mL, 7.38 mmol). Stirred reaction at room temperature for four hours. Concentrated solvent, and azeotroped hydrazine several times with methanol. Triturated solid with 1N HCl and then basified mother liquor with 1N NaOH. Washed aqueous layer with dichloromethane and concentrated to afford 0.74 g (89% yield) of [(3-bromoimidazo[1,2-a]pyridin-2-yl)methyl]amine with no further purification. ¹H NMR (400 MHz, DMSO-D6) δ 1.78 (br s, 2H), 3.75 (s, 2H), 7.03 (t, 1H), 7.31 (dd, 1H), 7.56 (m, 1H), 8.27 (m, 1H).

### c) N-[(3-bromoimidazo[1,2-a]pyridin-2-yl)methyl]-5,6,7,8-tetrahydro-8-quinolinamine

Dissolved [(3-bromoimidazo[1,2-a]pyridin-2-yl)methyl]amine (0.74 g, 3.29 mmol), 6,7-dihydro-8(5H)-quinolinone (0.48 g, 2.99 mmol), sodium triacetoxyborohydride (0.951 g, 4.48 mmol) and acetic acid (0.257 mL, 4.48 mmol) in 1,2-dichlorethane (10 mL). Reaction was stirred overnight at room temperature. Diluted reaction mixture with dichloromethane and stirred vigorously with 10% aqueous sodium carbonate for 30 minutes. Separated layers and washed with dichloromethane twice. Dried over magnesium sulfate and concentrated to afford 1.08 g (89% yield) of N-[(3-bromoimidazo[1,2-a]pyridin-2-yl)methyl]-5,6,7,8-tetrahydro-8-quinolinamine. ¹H NMR (400 MHz, DMSO-D6) δ 1.67 (m, 2H), 1.91 (m, 1H), 2.17 (m, 1H), 2.73 (m, 2H), 3.10 (s, 1H), 3.67 (t, 1H), 3.86 (d, 1H), 3.98 (d, 1H), 7.05 (t, 1H), 7.14 (dd, 1H), 7.33 (dd, 1H), 7.46 (d, 1H), 7.60 (d, 1H), 8.29 (d, 1H), 8.33 (d, 1H).

### d) N-[(3-bromoimidazo[1,2-a]pyridin-2-yl)methyl]-N-methyl-5,6,7,8-tetrahydro-8-quinolinamine

Dissolved N-[(3-bromoimidazo[1,2-a]pyridin-2-yl)methyl]-5,6,7,8-tetrahydro-8-quinolinamine (1.08 g, 3.03 mmol), formaldehyde (37% aqueous solution, 0.45 mL, 6.06 mmol), sodium triacetoxyborohydride (1.28 g, 6.06 mmol) and acetic acid (0.35 mL, 6.06 mmol) in 1,2-dichloroethane (10 mL). Reaction was stirred overnight at room temperature. Diluted reaction mixture with dichloromethane and stirred vigorously with 10% aqueous sodium carbonate for 15 minutes. Separated layers and washed with water twice. Dried over magnesium sulfate and concentrated. Residue was purified by silica gel chromatography on a 0-10% gradient of ammonium hydroxide in acetonitrile to afford 0.89 g (79% yield) of N-[(3-bromoimidazo[1,2-a]pyridin-2-yl)methyl]-N-methyl-5,6,7,8-tetrahydro-8-quinolinamine. ¹H NMR (400 MHz, DMSO-D4) δ 1.62 (m, 1H), 1.98 (m, 3H), 2.19 (s, 3H), 2.67 (m, 1H), 2.80 (m, 1H), 3.90 (m, 3H), 7.03 (t, 1H), 7.15 (dd, 1H), 7.30 (dd, 1H), 7.47 (d, 1H), 7.57 (d, 1H), 8.27 (d, 1H), 8.39 (d, 1H).

### e) N,N-dimethyl-N′-[(2-{[methyl(5,6,7,8-tetrahydro-8-quinolinyl)amino]methyl}imidazo[1,2-a]pyridin-3-yl)methyl]-1,2-ethanediamine (GSK650380A)

Dissolved N-[(3-bromoimidazo[1,2-a]pyridin-2-yl)methyl]-N-methyl-5,6,7,8-tetrahydro-8-quinolinamine (0.30 g, 0.810 mmol) in anhydrous tetrahydrofuran (3 mL) and cooled solution to −78° C. Added n-butyl lithium (1.6M solution in tetrahydrofuran, 0.36 mL, 0.891 mmol) dropwise keeping temperature below −70° C. Added anhydrous dimethylformamide (0.124 mL, 1.62 mmol) dropwise, again keeping temperature below −70° C. Reaction was slowly brought up to room temperature and stirred for four hours. Diluted reaction with water and dichloromethane, separated layers and washed with dichloromethane. Dried over magnesium sulfate and concentrated. Residue was purified by silica gel chromatography on a 0-10% gradient of ammonium hydroxide in acetonitrile to afford 0.15 g of a mixture of desired product and fully reduced by-product, which was further purified by reverse phase chromatography on a 0-30% gradient of water (0.1% trifluoroacetic acid) in acetonitrile to afford 0.044 g (17% yield) of 2-{[methyl(5,6,7,8-tetrahydro-8-quinolinyl)amino]methyl}imidazo[1,2-a]pyridine-3-carbaldehyde as the trifluoroacetic acid salt. Dissolved the 2-{[methyl(5,6,7,8-tetrahydro-8-quinolinyl)amino]methyl}imidazo[1,2-a]pyridine-3-carbaldehyde (0.044 g, 0.137 mmol), N,N-dimethyl-1,2-ethanediamine (0.023 mL, 0.206 mmol), acetic acid (0.012 mL, 0.206 mmol) and sodium triacetoxyborohydride (0.044 g, 0.206 mmol) in 1,2-dichloroethane (1.0 mL). Reaction was heated with a heat gun until boiling then diluted with dichloromethane and stirred vigorously with 10% aqueous sodium carbonate. Separated layers and washed with water and saturated aqueous sodium chloride. Dried organics over magnesium sulfate and concentrated. Residue was purified by reverse phase chromatography on a 0-100% gradient of water (0.1% trifluoroacetic acid) in acetonitrile to afford 0.021 g (39% yield) of N,N-dimethyl-N¹-[(2-{[methyl(5,6,7,8-tetrahydro-8-quinolinyl)amino]methyl}imidazo[1,2-a]pyridin-3-yl)methyl]-1,2-ethanediamine as the trifluoroacetic acid salt. ¹H NMR (300 MHz, DMSO-D6) δ 1.83 (m, 1H), 2.14 (m, 2H), 2.48 (m, 1H), 2.80 (s, 3H), 2.88 (s, 6H), 3.38 (m, 4H), 4.50 (m, 2H), 4.68 (m, 4H), 4.87 (m, 1H), 7.18 (t, 1H), 7.42 (dd, 1H), 7.52 (t, 1H), 7.73 (d, 2H), 8.57 (d, 1H), 8.76 (d, 1H); MS m/z 393 (M+1).

### Example 31

### N-methyl-N-({3-[(4-methyl-1-piperazinyl)methyl]imidazo[1,2-d]pyridin-2-yl}methyl)-5,6,7,8-tetrahydro-8-quinolinamine

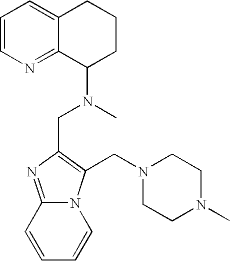

### a) 2-({3-[(4-methyl-1-piperazinyl)methyl]imidazo[1,2-a]pyridin-2-yl}methyl)-1H-isoindole-1,3(2H)-dione

Dissolved 2-[(1,3-dioxo-1,3-dihydro-2H-isoindol-2-yl)methyl]imidazo[1,2-a]pyridine-3-carbaldehyde (0.50 g, 1.65 mmol), acetic acid (0.141 mL, 2.47 mmol), 1-methylpiperazine (0.274 mL, 2.47 mmol) and sodium triacetoxyborohydride (0.044 g, 0.206 mmol) in 1,2-dichloroethane (10 mL). Reaction was heated with a heat gun until boiling for 30 minutes, then diluted with dichloromethane and stirred vigorously with 10% aqueous sodium carbonate overnight. Separated layers and washed with water and saturated aqueous sodium chloride. Dried organics over magnesium sulfate and concentrated to afford 0.63 g (98% yield) of 2-({3-[(4-methyl-1-piperazinyl)methyl]imidazo[1,2-a]pyridin-2-yl}methyl)-1H-isoindole-1,3(2H)-dione. ¹H NMR (300 MHz, DMSO-D6) δ 2.12 (5, 3H), 2.23 (m, 4H), 2.43 (m, 4H), 3.90 (s, 2H), 4.95 (s, 2H), 6.94 (t, 1H), 7.24 (t, 1H), 7.47 (d, 1H), 7.91 (m, 4H), 8.42 (d, 1H).

### b) ({3-[(4-methyl-1-piperazinyl)methyl]imidazo[1,2-a]pyridin-2-yl}methyl)amine

Dissolved 2-({3-[(4-methyl-1-piperazinyl)methyl]imidazo[1,2-a]pyridin-2-yl}methyl)-1H-isoindole-1,3(2H)-dione (0.628 g, 1.61 mmol) in anhydrous methanol (5 mL) and added hydrazine (0.101 mL, 3.23 mmol). Stirred reaction at room temperature for three hours. Concentrated solvent, and azeotroped hydrazine several times with methanol. Triturated solid with 1N HCl and then basified mother liquor with 1N NaOH. Washed aqueous layer with dichloromethane and concentrated to afford 0.246 g (59% yield) of ({3-[(4-methyl-1-piperazinyl)methyl]imidazo[1,2-a]pyridin-2-yl}methyl)amine with no further purification. ¹H NMR (400 MHz, DMSO-D6) δ 1.74 (m, 1H), 2.10 (s, 3H), 2.24 (m, 3H), 3.75 (s, 2H), 0.77 (s, 2H), 6.88 (t, 1H), 7.20 (t, 1H), 7.46 (d, 1H), 8.33 (d, 1H).

### c) N-({3-[(4-methyl-1-piperazinyl)methyl]imidazo[1,2-a]pyridin-2-yl}methyl)-5,6,7,8-tetrahydro-8-quinolinamine

Dissolved ({3-[(4-methyl-1-piperazinyl)methyl]imidazo[1,2-a]pyridin-2-yl}methyl)amine (0.245 g, 0.944 mmol), acetic acid (0.108 mL, 1.89 mmol), 6,7-dihydro-8(5H)-quinolinone as prepared herein (0.139 g, 0.944 mmol) and sodium triacetoxyborohydride (0.40 g, 1.89 mmol) in 1,2-dichloroethane (5 mL). Reaction was heated with a heat gun until boiling, then diluted with dichloromethane and stirred vigorously with 10% aqueous sodium carbonate for 30 minutes. Separated layers and washed with water and saturated aqueous sodium chloride. Dried organics over magnesium sulfate and concentrated to afford 0.31 g (85% yield) of N-({3-[(4-methyl-1-piperazinyl)methyl]imidazo[1,2-a]pyridin-2-yl}methyl)-5,6,7,8-tetrahydro-8-quinolinamine with no further purification. ¹H NMR (300 MHz, DMSO-D6) δ 1.73 (m, 2H), 1.95 (m, 1H), 2.15 (s, 3H), 2.29 (m, 3H), 2.43 (m, 4H), 2.78 (m, 2H), 3.78 (m, 1H), 3.86 (d, 2H), 3.93 (s, 4H), 4.04 (d, 1H), 6.93 (t, 1H), 7.21 (m, 2H), 7.53 (t, 2H), 8.39 (m, 2H); MS m/z 412 (M+Na).

### d): N-methyl-N-({3-[(4-methyl-1-piperazinyl)methyl]imidazo[1,2-a]pyridin-2-yl}methyl)-5,6,7,8-tetrahydro-8-quinolinamine

Dissolved N-({3-[(4-methyl-1-piperazinyl)methyl]imidazo[1,2-a]pyridin-2-yl}methyl)-5,6,7,8-tetrahydro-8-quinolinamine (0.10 g, 0.257 mmol), formaldehyde (37% aqueous solution, 0.019 mL, 0.257 mmol), sodium triacetoxyborohydride (0.081 g, 0.385 mmol) and acetic acid (0.022 mL, 0.385 mmol) in 1,2-dichlorethane (2 mL). Reaction was stirred overnight at room temperature. Diluted reaction mixture with dichloromethane and stirred vigorously with 10% aqueous sodium carbonate for 15 minutes. Separated layers and washed with water twice. Dried over magnesium sulfate and concentrated. Residue was purified by silica gel chromatography on a 0-10% gradient of ammonium hydroxide in acetonitrile to afford 0.078 g (76% yield) of N-methyl-N-({3-[(4-methyl-1-piperazinyl)methyl]imidazo[1,2-a]pyridin-2-yl}methyl)-5,6,7,8-tetrahydro-8-quinolinamine. ¹H NMR (400 MHz, DMSO-D4) δ 1.62 (m, 1H), 1.88 (m, 1H), 1.98 (m, 2H), 2.06 (s, 2H), 2.10 (s, 3H), 2.13 (s, 3H), 2.28 (m, 6H), 2.69 (m, 1H), 2.78 (m, 1H), 3.78 (m, 4H), 3.93 (d, 1H), 6.86 (t, 1H), 7.18 (m, 2H), 7.48 (m, 2H), 8.33 (d, 1H), 8.41 (dd, 1H); MS m/z 405 (M+1).

### Example 32

### N-[(2-{-[methyl(5,6,7,8-tetrahydro-8-quinolinyl)amino]methyl}imidazo[1,2-a]pyridin-3-yl)methyl]-1,3-propanediamine

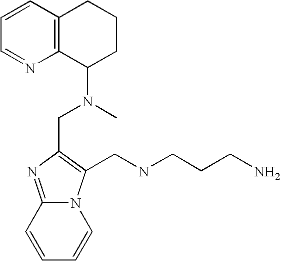

### a) 1,1-dimethylethyl{3-[({2-[(1,3-dioxo-1,3-dihydro-2H-isoindol-2-yl)methyl]imidazo[1,2-a]pyridin-3-yl}methyl)amino]propyl}carbamate

Dissolved 2-[(1,3-dioxo-1,3-dihydro-2H-isoindol-2-yl)methyl]imidazo[1,2-a]pyridine-3-carbaldehyde (0.20 g, 0.656 mmol), acetic acid (0.056 mL, 0.983 mmol), 1,1-dimethylethyl (3-aminopropyl)carbamate (0.226 g, 1.31 mmol) and sodium triacetoxyborohydride (0.416 g, 1.97 mmol) in 1,2-dichloroethane (4 mL). Stirred at room temperature overnight. Diluted with dichloromethane and 10% aqueous sodium carbonate. Separated layers and washed with water and saturated aqueous sodium chloride. Dried organics over magnesium sulfate and concentrated to afford 0.28 g (91% yield) of 1,1-dimethylethyl{3-[({2-[(1,3-dioxo-1,3-dihydro-2H-isoindol-2-yl)methyl]imidazo[1,2-a]pyridin-3-yl}methyl)amino]propyl}carbamate with no further purification. ¹H NMR (400 MHz, DMSO-D6) δ 1.35 (m, 9H), 1.50 (m, 2H), 2.94 (m, 2H), 3.89 (s, 2H), 4.09 (s, 2H), 4.91 (s, 2H), 6.77 (t, 1H), 6.87 (t, 1H), 7.41 (d, 1H), 7.85 (m, 41-1), 8.40 (d, 1H); MS m/z 463 (M+1)

### b) 1,1-dimethylethyl[3-({[(1,1-dimethylethyl)oxy]carbonyl}amino)propyl]({2-[(1,3-dioxo-1,3-dihydro-2H-isoindol-2-yl)methyl]imidazo[1,2-a]pyridin-3-yl}methyl)carbamate

Dissolved 1,1-dimethylethyl {3-[({2-[(1,3-dioxo-1,3-dihydro-2H-isoindol-2-yl)methyl]imidazo[1,2-a]pyridin-3-yl}methyl)amino]propyl}carbamate (0.28 g, 0.594 mmol) in anhydrous dichloromethane (10 mL). Added bis(1,1-dimethylethyl) dicarbonate (0.39 g, 1.78 mmol), dimethylaminopyridine (0.007 g, 0.059 mmol) and diisopropylethylamine (0.207 mL, 1.187 mmol) and let stir at room temperature for 6 hours. Diluted with dichloromethane and washed with twice with water and saturated aqueous sodium chloride. Dried organics over magnesium sulfate and concentrated to 0.33 g (99% yield) of 1,1-dimethylethyl[3-({[(1,1-dimethylethyl)oxy]carbonyl}amino)propyl]({2-[(1,3-dioxo-1,3-dihydro-2H-isoindol-2-yl)methyl]imidazo[1,2-a]pyridin-3-yl}methyl)carbamate with no further purification. ¹H NMR (400 MHz, DMSO-D6) δ 1.30 (s, 9H), 1.34 (m, 2H), 1.42 (s, 9H), 2.84 (m, 2H), 3.05 (m, 2H), 4.89 (s, 2H), 4.94 (s, 2H), 4.91 (s, 2H), 6.72 (m, 1H), 6.93 (m, 1H), 7.19 (m, 1H), 7.44 (d, 1H), 7.86 (m, 4H), 8.31 (m, 1H); MS m/z 564 (M+1).

### c) 1,1-dimethylethyl{[2-(aminomethyl)imidazo[1,2-a]pyridin-3-yl]methyl}[3-({[(1,1-dimethylethyl)oxy]carbonyl}amino)propyl]carbamate

Dissolved 1,1-dimethylethyl[3-({[(1,1-dimethylethyl)oxy]carbonyl}amino)propyl]({2-[(1,3-dioxo-1,3-dihydro-2H-isoindol-2-yl)methyl]imidazo[1,2-a]pyridin-3-yl}methyl)carbamate (0.35 g, 0.62 mmol) in anhydrous methanol (5 mL) and added hydrazine (0.039 mL, 1.25 mmol). Stirred reaction at room temperature overnight. Concentrated solvent, and azeotroped hydrazine several times with methanol. Added acetonitrile and filtered off by-product. Concentrated filtrate and purified by reverse phase chromatography on a 1-100% gradient of water (0.1% trifluoroacetic acid) in acetonitrile. Recovered 0.10 g (37% yield) of 1,1-dimethylethyl {[2-(aminomethyl)imidazo[1,2-a]pyridin-3-yl]methyl}[3-({[(1,1-dimethylethyl)oxy]carbonyl}amino)propyl]carbamate. ¹H NMR (400 MHz, DMSO-D6) δ 1.33 (s, 9H), 1.37 (m, 2H), 1.42 (s, 9H), 2.79 (m, 2H), 2.98 (m, 2H), 4.25 (d, 2H), 4.83 (s, 2H), 4.91 (s, 21-1), 6.72 (m, 1H), 7.10 (t, 1H), 7.41 (dd, 1H), 7.65 (d, 1H), 8.29 (m, 1H); MS m/z 434 (M+1).

### d) N-[(2-{[methyl(5,6,7,8-tetrahydro-8-quinolinyl)amino]methyl}imidazo[1,2-a]pyridin-3-yl)methyl]-1,3-propanediamine

Dissolved 1,1-dimethylethyl{[2-(aminomethyl)imidazo[1,2-a]pyridin-3-yl]methyl}[3-({[(1,1-dimethylethyl)oxy]carbonyl}amino)propyl]carbamate (0.10 g, 0.231 mmol), acetic acid (0.026 mL, 0.462 mmol), 6,7-dihydro-8(5H)-quinolinone as prepared herein (0.034 g, 0.231 mmol) and sodium triacetoxyborohydride (0.098 g, 0.46 mmol) in 1,2-dichloroethane (5 mL). Stirred at room temperature overnight. Diluted with dichloromethane and 10% aqueous sodium carbonate. Separated layers and washed with water and saturated aqueous sodium chloride. Dried organics over magnesium sulfate and concentrated. Residue was purified by reverse phase chromatography on a 0-100% gradient of water (0.1% trifluoroacetic acid) in acetonitrile to afford 0.060 g (46% yield) of 1,1-dimethylethyl[3-({[(1,1-dimethylethyl)oxy]carbonyl}amino)propyl]({2-[(5,6,7,8-tetrahydro-8-quinolinylamino)methyl]imidazo[1,2-a]pyridin-3-yl}methyl)carbamate as the trifluoroacetic acid salt. Dissolved this product (0.08 g, 0.141 mmol), formaldehyde (37% aqueous solution, 0.011 mL, 0.141 mmol), and sodium triacetoxyborohydride (0.045 g, 0.212 mmol) in 1,2-dicholorethane (2 mL). Reaction was stirred overnight at room temperature. Diluted reaction mixture with dichloromethane and stirred vigorously with 10% aqueous sodium carbonate for 15 minutes. Separated layers and washed with water twice. Dried over magnesium sulfate and concentrated. Residue was purified by reverse phase chromatography on a 0-100% gradient of water (0.1% trifluoroacetic acid) in acetonitrile to afford 0.76 g (95% yield) of 1,1-dimethylethyl[3-({[(1,1-dimethylethyl)oxy]carbonyl}amino)propyl][(2-{[methyl(5,6,7,8-tetrahydro-8-quinolinyl)amino]methyl}imidazo[1,2-a]pyridin-3-yl)methyl]carbamate as the trifluoroacetic acid salt. Dissolved this product in dichloromethane (5 mL) and added trifluoroacetic acid (5 mL). Let stir at room temperature overnight. Concentrated solvent and purified by reverse phase chromatography on a 0-100% gradient of water (0.1% trifluoroacetic acid) in acetonitrile to afford 0.04 g (79% yield) of N-[(2-{[methyl(5,6,7,8-tetrahydro-8-quinolinyl)amino]methyl}imidazo[1,2-a]pyridin-3-yl)methyl]-1,3-propanediamine as the trifluoroacetic acid salt. ¹H NMR (300 MHz, DMSO-D6) δ 1.83 (m, 1H), 1.95 (m, 2H), 2.14 (m, 2H), 2.47 (m, 1H), 2.80 (s, 3H), 2.89 (m, 4H), 3.15 (t, 2H), 4.50 (d, 1H), 4.62 (d, 1H), 4.76 (m, 2H), 4.88 (m, 1H), 7.16 (t, 1H), 7.40 (dd, 1H), 7.51 (t, 1H), 7.72 (d, 2H), 8.56 (d, 1H), 8.76 (d, 1H); MS m/z 379 (M+1).

### Example 33

### N-[2-(dimethylamino)ethyl]-2-{[methyl(5,6,7,8-tetrahydro-8-quinolinyl)amino]methyl}imidazo[1,2-a]pyridine-3-carboxamide

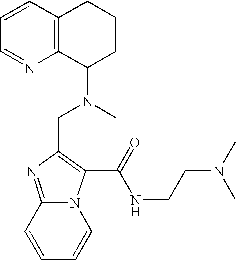

### a) Ethyl 2-methylimidazo[1,2-a]pyridine-3-carboxylate

Dissolved 2-aminopyridine (2.0 g, 21.2 mmol) in absolute ethanol (100 mL) and added ethyl 2-chloro-3-oxobutanoate (2.7 mL, 19.5 mmol). Refluxed reaction overnight, then concentrated solvent and brought up residue in dichloromethane. Extracted with water twice, followed by a wash with saturated aqueous sodium chloride. Dried over magnesium sulfate and concentrated. Purified by silica gel chromatography on a 0-7% of 2N methanolic ammonia in dichloromethane to afford 2.0 g (46% yield) of ethyl 2-methylimidazo[1,2-a]pyridine-3-carboxylate. ¹H NMR (400 MHz, DMSO-D6) δ 1.32 (t, 3H), 2.56 (s, 3H), 4.32 (q, 2H), 7.13 (t, 1H), 7.48 (t, 1H), 7.63 (d, 1H), 9.17 (d, 1H); MS m/z 205 (M+1).

### b) Ethyl 2-(bromomethyl)imidazo[1,2-a]pyridine-3-carboxylate

Dissolved ethyl 2-methylimidazo[1,2-a]pyridine-3-carboxylate (1.0 g, 4.90 mmol) in carbon tetrachloride (65 mL). Added N-bromosuccinamide (recrystallized from water, 0.872 g, 4.90 mmol) and 2,2′-azobisisobutyronitrile (0.04 g, 0.24 mmol). Heated reaction to reflux overnight. Cooled to room temperature and filtered. Concentrated filtrate and purified by silica gel chromatography on a 30-50% gradient of ethyl acetate in hexane to afford 0.47 g (33% yield) of ethyl 2-(bromomethyl)imidazo[1,2-a]pyridine-3-carboxylate. ¹H NMR (400 MHz, DMSO-D6) δ 1.39 (t, 3H), 4.40 (q, 3H), 4.93 (s, 2H), 7.25 (t, 1H), 7.60 (t, 1H), 7.77 (d, 1H), 9.21 (d, 1H); MS m/z 283 (M+1).

### c) Ethyl 2-{[methyl(5,6,7,8-tetrahydro-8-quinolinyl)amino]methyl}imidazo[1,2-a]pyridine-3-carboxylate

Dissolved ethyl 2-(bromomethyl)imidazo[1,2-a]pyridine-3-carboxylate (0.93 g, 3.3 mmol) in acetonitrile (50 mL). Added N-methyl-5,6,7,8-tetrahydro-8-quinolinamine (0.53 g, 3.3 mmol), diisopropylethylamine (0.63 mL, 3.63 mmol), and potassium iodide (0.27 g, 1.65 mmol). Stirred vigorously for one hour. Concentrated and residue was dissolved in dichloromethane. Washed with water twice, followed by saturated aqueous sodium chloride. Dried over magnesium sulfate and concentrated. Purified by silica gel chromatography on a 0-10% gradient of ammonium hydroxide in acetonitrile to afford 0.74 g (62% yield) of ethyl 2-{[methyl(5,6,7,8-tetrahydro-8-quinolinyl)amino]methyl}imidazo[1,2-a]pyridine-3-carboxylate. ¹H NMR (400 MHz, DMSO-D6) δ 1.32 (t, 3H), 1.98 (m, 1H), 1.63 (m, 1H), 1.92 (m, 3H), 2.20 (s, 3H), 2.66 (m, 1H), 2.76 (m, 1H), 4.03 (t, 1H), 4.27 (s, 2H), 4.33 (q, 2H), 7.16 (m, 2H), 7.46 (d, 1H), 7.17 (t, 1H), 7.72 (d, 1H), 8.37 (d, 1H), 9.24 (d, 1H); MS m/z 365 (M+1).

### d) 2-{[methyl(5,6,7,8-tetrahydro-8-quinolinyl)amino]methyl}imidazo[1,2-a]pyridine-3-carboxylic acid

Dissolved ethyl 2-{[methyl(5,6,7,8-tetrahydro-8-quinolinyl)amino]methyl}imidazo[1,2-a]pyridine-3-carboxylate (0.74 g, 2.03 mmol) in methanol (75 mL) and added sodium hydroxide (1N aqueous solution, 9.15 mL, 9.15 mmol) and let react at room temperature overnight. Concentrated solvent and purified residue by silica gel chromatography on a 0-10% gradient of ammonium hydroxide in acetonitrile to afford a quantitative yield of 2-{[methyl(5,6,7,8-tetrahydro-8-quinolinyl)amino]methyl}imidazo[1,2-a]pyridine-3-carboxylic acid. ¹H NMR (400 MHz, DMSO-D6) δ 1.57 (m, 1H), 1.96 (m, 2H), 2.08 (m, 4H), 2.66 (m, 1H), 2.77 (m, 1H), 3.14 (s, 1H), 3.93 (t, 1H), 4.08 (d, 1H), 4.28 (d, 1H), 6.83 (t, 1H), 7.16 (m, 2H), 7.45 (m, 2H), 8.32 (d, 1H), 9.73 (d, 1H); MS m/z 337 (M+1).

### e) N-[2-(dimethylamino)ethyl]-2-{[methyl(5,6,7,8-tetrahydro-8-quinolinyl)amino]methyl}imidazo[1,2-a]pyridine-3-carboxamide

Dissolved 2-{[methyl(5,6,7,8-tetrahydro-8-quinolinyl)amino]methyl}imidazo[1,2-a]pyridine-3-carboxylic acid (0.1 g, 0.28 mmol) in N,N-dimethylformamide (5 mL) and added N,N-dimethyl-1,2-ethanediamine (0.032 mL, 0.29 mmol), 1-hydroxybenzotriazole hydrate (0.16 g, 1.19 mmol), and 1-(3-dimethylaminopropyl)-3-ethylcarbodiimide hydrochloride (0.11 g, 0.59 mmol). Reaction complete after 24 hours at room temperature. Diluted reaction with dichloromethane and washed two times with water, and once with saturated aqueous sodium chloride. Dried organics over magnesium sulfate and concentrated. Residue was purified by reverse phase chromatography on a 0-100% gradient of acetonitrile in water (0.1% trifluoroacetic acid) to afford 0.042 g (35% yield) of N-[2-(dimethylamino)ethyl]-2-{[methyl(5,6,7,8-tetrahydro-8-quinolinyl)amino]methyl}imidazo[1,2-a]pyridine-3-carboxamide as the trifluoroacetic acid salt. ¹H NMR (400 MHz, DMSO-D6) δ 1.81 (m, 1H), 2.08 (m, 2H), 2.41 (m, 1H), 2.74 (m, 3H), 2.83 (m, 1H), 2.92 (m, 9H), 3.67 (m, 2H), 4.53 (d, 1H), 4.66 (d, 1H), 4.77 (m, 1H), 7.22 (t, 1H), 7.40 (dd, 1H), 7.58 (t, 1H), 7.72 (d, 1H), 7.79 (d, 1H), 8.51 (d, 1H), 9.03 (d, 1H), 9.44 (br s, 1H); MS m/z 407 (M+1).

### Example 34

### N-({3-[(3-amino-1-azetidinyl)carbonyl]imidazo[1,2-a]pyridin-2-yl}methyl)-N-methyl-5,6,7,8-tetrahydro-8-quinolinamine

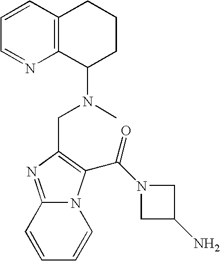

Dissolved 2-{[methyl(5,6,7,8-tetrahydro-8-quinolinyl)amino]methyl}imidazo[1,2-a]pyridine-3-carboxylic acid (0.1 g, 0.28 mmol) in N,N-dimethylformamide (5 mL) and added 1,1-dimethylethyl 3-azetidinylcarbamate (0.05 g, 0.297 mmol), 1-hydroxybenzotriazole hydrate (0.16 g, 1.19 mmol), and 1-(3-dimethylaminopropyl)-3-ethylcarbodiimide hydrochloride (0.11 g, 0.59 mmol). Reaction complete after 24 hours at room temperature. Diluted reaction with dichloromethane and washed two times with water, and once with saturated aqueous sodium chloride. Dried organics over magnesium sulfate and concentrated. Residue was purified by reverse phase chromatography on a 0-100% gradient of acetonitrile in water (0.1% trifluoroacetic acid) to afford 0.028 g (19% yield) of 1,1-dimethylethyl {1-[(2-{[methyl(5,6,7,8-tetrahydro-8-quinolinyl)amino]methyl}imidazo[1,2-a]pyridin-3-yl)carbonyl]-3-azetidinyl}carbamate as the trifluoroacetic acid salt which was dissolved in dichloromethane (3 mL) and trifluoroacetic acid (1.5 mL) was added to remove the protecting group. Reaction complete after two hours. Evaporated solvent to afford N-({3-[(3-amino-1-azetidinyl)carbonyl]imidazo[1,2-a]pyridin-2-yl}methyl)-N-methyl-5,6,7,8-tetrahydro-8-quinolinamine in quantitative yield. ¹H NMR (300 MHz, DMSO-D6) δ 1.80 (m, 1H), 2.09 (m, 2H), 2.43 (m, 1H), 2.85 (m, 6H), 4.09 (m, 3H), 4.33 (m, 2H), 4.55 (d, 1H), 4.68 (d, 1H), 4.83 (m, 1H), 7.25 (t, 1H), 7.42 (dd, 1H), 7.59 (t, 1H), 7.73 (m, 2H), 7.82 (d, 1H), 8.52 (d, 1H), 8.67 (d, 1H); MS m/z 391 (M+1).

### Example 35

### 2-{[methyl(5,6,7,8-tetrahydro-8-quinolinyl)amino]methyl}-N-[2-(1-pyrrolidinyl)ethyl]imidazo[1,2-a]pyridine-3-carboxamide

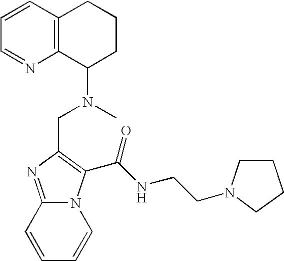

Dissolved 2-{[methyl(5,6,7,8-tetrahydro-8-quinolinyl)amino]methyl}imidazo[1,2-a]pyridine-3-carboxylic acid (0.1 g, 0.28 mmol) in N,N-dimethylformamide (5 mL) and added [2-(1-pyrrolidinyl)ethyl]amine (0.032 mL, 0.30 mmol), 1-hydroxybenzotriazole hydrate (0.16 g, 1.19 mmol), and 1-(3-dimethylaminopropyl)-3-ethylcarbodiimide hydrochloride (0.11 g, 0.59 mmol). Reaction complete after 24 hours at room temperature. Diluted reaction with dichloromethane and washed two times with water, and once with saturated aqueous sodium chloride. Dried organics over magnesium sulfate and concentrated. Residue was purified by reverse phase chromatography on a 0-100% gradient of acetonitrile in water (0.1% trifluoroacetic acid) to afford 0.032 g (25% yield) of 2-{[methyl(5,6,7,8-tetrahydro-8-quinolinyl)amino]methyl}-N-[2-(1-pyrrolidinyl)ethyl]imidazo[1,2-a]pyridine-3-carboxamide as the trifluoroacetic acid salt. ¹H NMR (400 MHz, DMSO-D6) δ 1.73 (m, 1H), 1.85 (m, 2H), 2.00 (m, 4H), 2.35 (m, 2H), 2.63 (m, 2H), 2.80 (m, 2H), 3.06 (m, 2H), 3.61 (m, 6H), 4.46 (m, 1H), 4.58 (m, 1H), 4.67 (m, 1H), 7.16 (t, 1H), 7.33 (dd, 1H), 7.51 (t, 1H), 7.65 (d, 1H), 7.72 (d, 1H), 8.45 (d, 1H), 8.98 (br s, 1H), 9.54 (br s, 1H); MS m/z 433 (M+1).

### Example 36

### N-[(3-{[3-(dimethylamino)-1-pyrrolidinyl]carbonyl}imidazo[1,2-a]pyridin-2-yl)methyl]-N-methyl-5,6,7,8-tetrahydro-8-quinolinamine

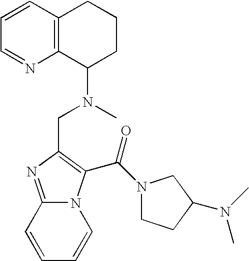

Dissolved 2-{[methyl(5,6,7,8-tetrahydro-8-quinolinyl)amino]methyl}imidazo[1,2-a]pyridine-3-carboxylic acid (0.1 g, 0.28 mmol) in N,N-dimethylformamide (5 mL) and added N,N-dimethyl-3-pyrrolidinamine (0.039 mL, 0.30 mmol), 1-hydroxybenzotriazole hydrate (0.16 g, 1.19 mmol), and 1-(3-dimethylaminopropyl)-3-ethylcarbodiimide hydrochloride (0.11 g, 0.59 mmol). Reaction complete after 24 hours at room temperature. Diluted reaction with dichloromethane and washed two times with water, and once with saturated aqueous sodium chloride. Dried organics over magnesium sulfate and concentrated. Residue was purified by reverse phase chromatography on a 0-100% gradient of acetonitrile in water (0.1% trifluoroacetic acid) to afford 0.015 g (12% yield) N-[(3-{[3-(dimethylamino)-1-pyrrolidinyl]carbonyl}imidazo[1,2-a]pyridin-2-yl)methyl]-N-methyl-5,6,7,8-tetrahydro-8-quinolinamine as the trifluoroacetic acid salt. ¹H NMR (400 MHz, DMSO-D6) δ 1.73 (m, 1H), 2.01 (m, 2H), 2.33 (m, 2H), 2.81 (m, 7H), 2.88 (s, 6H), 3.04 (m, 1H), 3.28 (m, 1H), 3.70 (m, 2H), 4.43 (m, 1H), 4.54 (m, 1H), 4.77 (m, 1H), 7.16 (t, 1H), 7.36 (dd, 1H), 7.50 (t, 1H), 7.68 (d, 1H), 7.73 (d, 1H), 8.43 (m, 2H); MS m/z 433 (M+1).

### Example 37

### N-methyl-N-({3-[(4-methyl-1-piperazinyl)carbonyl]imidazo[1,2-a]pyridin-2-yl}methyl)-5,6,7,8-tetrahydro-8-quinolinamine

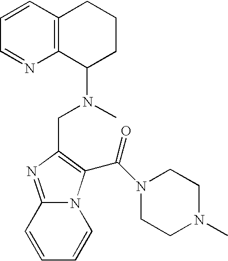

Dissolved 2-{[methyl(5,6,7,8-tetrahydro-8-quinolinyl)amino]methyl}imidazo[1,2-a]pyridine-3-carboxylic acid (0.1 g, 0.28 mmol) in N,N-dimethylformamide (5 mL) and added 1-methylpiperazine (0.033 mL, 0.30 mmol), 1-hydroxybenzotriazole hydrate (0.16 g, 1.19 mmol), and 1-(3-dimethylaminopropyl)-3-ethylcarbodiimide hydrochloride (0.11 g, 0.59 mmol). Reaction complete after 24 hours at room temperature. Diluted reaction with dichloromethane and washed two times with water, and once with saturated aqueous sodium chloride. Dried organics over magnesium sulfate and concentrated. Residue was purified by reverse phase chromatography on a 0-100% gradient of acetonitrile in water (0.1% trifluoroacetic acid) to afford 0.068 g (55% yield) N-methyl-N-({3-[(4-methyl-1-piperazinyl)carbonyl]imidazo[1,2-a]pyridin-2-yl}methyl)-5,6,7,8-tetrahydro-8-quinolinamine as the trifluoroacetic acid salt. ¹H NMR (400 MHz, DMSO-D6) δ 1.80 (m, 1H), 2.08 (m, 2H), 2.43 (m, 1H), 2.81 (m, 7H), 2.86 (m, 7H), 3.42 (m, 6H), 4.10 (m, 3H), 4.53 (m, 1H), 4.63 (m, 1H), 4.81 (m, 1H), 7.22 (t, 1H), 7.43 (dd, 1H), 7.57 (t, 1H), 7.74 (d, 1H), 7.81 (d, 1H), 8.54 (m, 2H); MS m/z 419 (M+1).

### Example 38

### N-methyl-N-[(3-{[4-(1-methylethyl)-1-piperazinyl]carbonyl}imidazo[1,2-a]pyridin-2-yl)methyl]-5,6,7,8-tetrahydro-8-quinolinamine

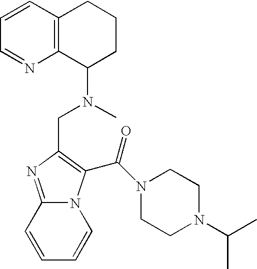

Dissolved 2-{[methyl(5,6,7,8-tetrahydro-8-quinolinyl)amino]methyl}imidazo[1,2-a]pyridine-3-carboxylic acid (0.1 g, 0.28 mmol) in N,N′-dimethylformamide (5 mL) and added 1-(1-methylethyl)piperazine (0.032 mL, 0.30 mmol), 1-hydroxybenzotriazole hydrate (0.16 g, 1.19 mmol), and 1-(3-dimethylaminopropyl)-3-ethylcarbodiimide hydrochloride (0.11 g, 0.59 mmol). Reaction complete after 24 hours at room temperature. Diluted reaction with dichloromethane and washed two times with water, and once with saturated aqueous sodium chloride. Dried organics over magnesium sulfate and concentrated. Residue was purified by reverse phase chromatography on a 0-100% gradient of acetonitrile in water (0.1% trifluoroacetic acid) to afford 0.067 g (51% yield) N-methyl-N-[(3-{[4-(1-methylethyl)-1-piperazinyl]carbonyl}imidazo[1,2-a]pyridin-2-yl)methyl]-5,6,7,8-tetrahydro-8-quinolinamine as the trifluoroacetic acid salt. ¹H NMR (300 MHz, DMSO-D6) δ 1.29 (s, 6H), 1.80 (m, 1H), 2.07 (m, 2H), 2.43 (m, 1H), 2.87 (m, 7H), 3.12 (m, 1H), 3.43 (m, 3H), 3.56 (m, 1H), 4.11 (m, 2H), 4.46 (m, 1H), 4.62 (m, 1H), 4.84 (m, 1H), 7.23 (t, 1H), 7.43 (dd, 1H), 7.57 (t, 1H), 7.74 (d, 1H), 7.81 (d, 1H), 8.54 (d, 1H), 8.60 (d, 1H); MS m/z 447 (M+1).

### Biological Section

### HOS HIV-1 Infectivity Assay

### HIV Virus Preparation

Compounds were profiled against two HIV-1 viruses, the M-tropic (CCR5 utilizing) Ba-L strain and the T-tropic (CXCR4 utilizing) IIIB strain. Ba-L was propagated in either peripheral blood lymphocytes or SupT1/CCR5+/CXCR4+ cells. IIIB was propagated in peripheral blood lymphocytes. Compounds were tested for their ability to block infection of the HOS cell line (expressing hCXCR4/hCCR5/hCD4/pHIV-LTR-luciferase) by either HIV-1 Ba-L or HIV-1 IIIB. Compound cytotoxicity was also examined in the absence of virus addition.

### HOS HIV-1 Infectivity Assay Format

HOS cells (expressing hCXCR4/hCCR5/hCD4/pHIV-LTR-luciferase) were harvested and diluted in Dulbeccos modified Eagles media supplemented with 2% FCS to a concentration of 120,000 cells/ml. The cells were plated into 96-well plates (50 μl per well) and the plates were placed in a tissue culture incubator (370° C.; 5% CO₂/95% air) for a period of 24 h.

Subsequently, 50 μl of the desired drug solution (2 times the final concentration) was added to each well and the plates were returned to the tissue culture incubator (370° C.; 5% CO₂/95% air) for 1 h. Following this incubation, 60 μl of diluted virus (4 times the final concentration) was added to 60 μl of 2× the final desired concentration of the compound and 100 μl this compound/virus mix was added to each well (approximately 2 million RLU per well of virus). The plates were returned to the tissue culture incubator (370° C.; 5% CO₂/95% air) and were incubated for a further 96 h.

Following this incubation the endpoint for the virally infected cultures was quantified following addition of Steady-Glo Luciferase assay system reagent (Promega, Madison, Wis.). Cell viability or non-infected cultures was measured using a CellTiter-Glo luminescent cell viability assay system (Promega, Madison, Wis.). All luminescent readouts are performed on a Topcount luminescence detector (Packard, Meridien, Conn.).

TABLE 1

| Example | Structure | Activity Level* |
|---|---|---|
| 1 | 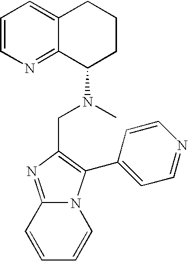 | A |
| 2 | 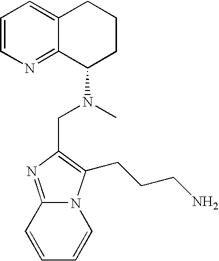 | A |
| 3 | 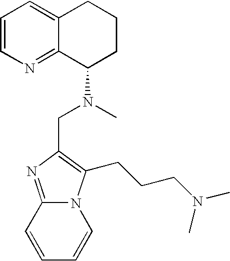 | A |
| 4 | 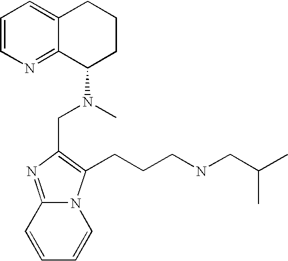 | A |
| 5 | 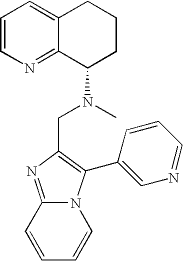 | B |
| 6 | 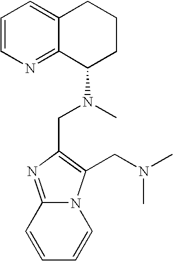 | A |
| 7 | 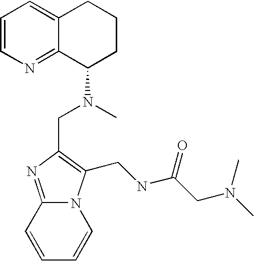 | A |
| 8 | 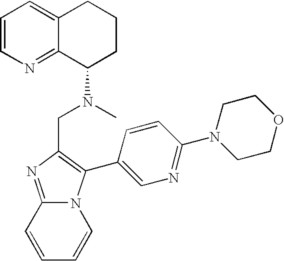 | B |
| 9 | 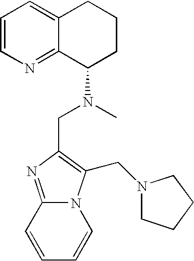 | A |
| 10 | 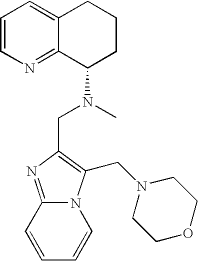 | B |
| 11 | 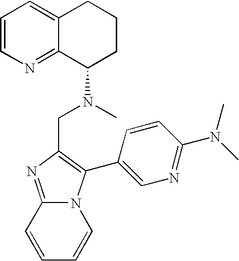 | A |
| 12 | 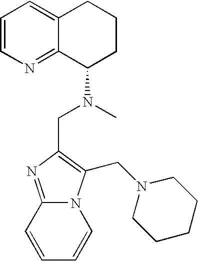 | A |
| 13 | 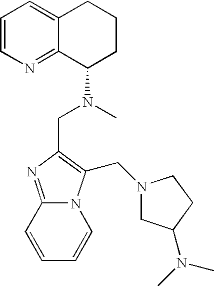 | A |
| 14 | 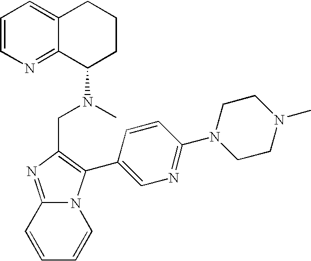 | A |
| 15 | 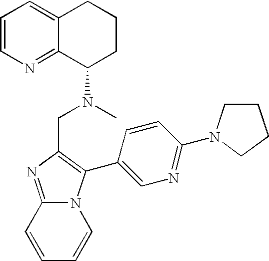 | B |
| 16 | 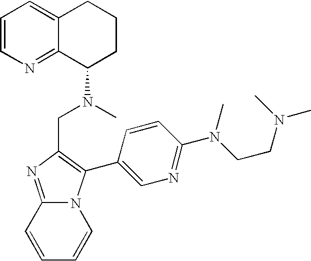 | C |
| 17 | 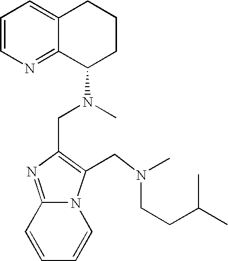 | A |
| 18 | 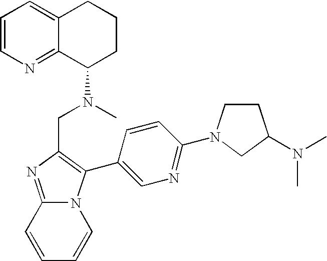 | C |
| 19 | 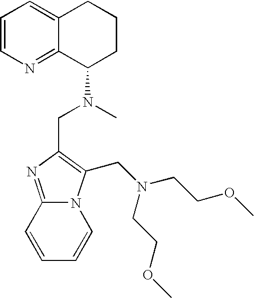 | B |
| 20 | 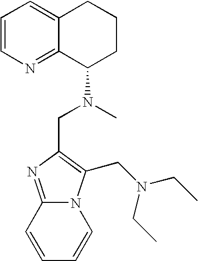 | B |
| 21 | 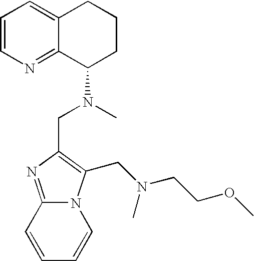 | B |
| 22 | 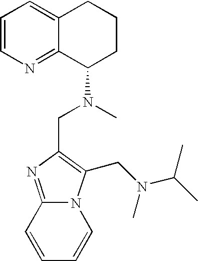 | A |
| 23 | 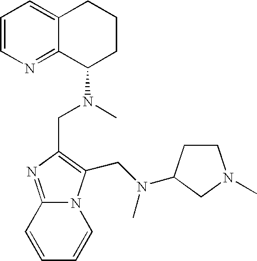 | A |
| 24 | 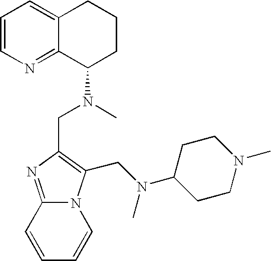 | A |
| 25 | 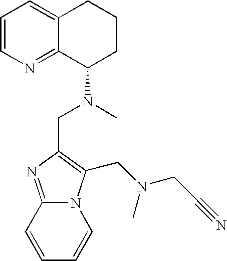 | A |
| 26 | 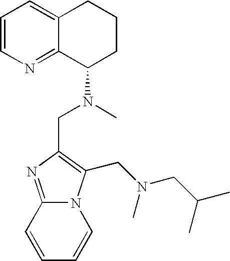 | B |
| 27 | 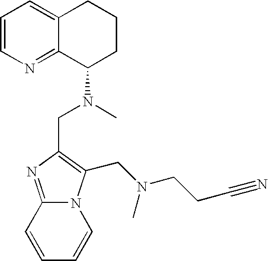 | A |
| 28 | 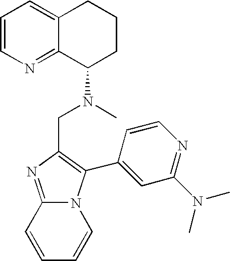 | B |
| 29 | 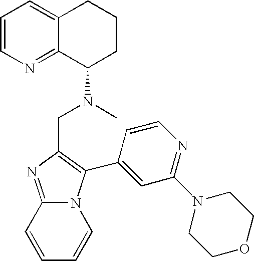 | B |
| 30 | 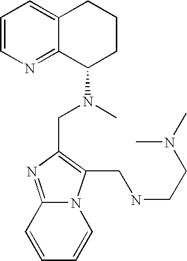 | B |
| 31 | 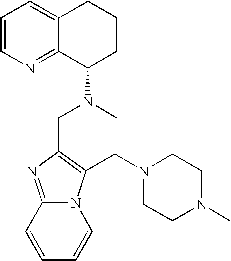 | B |
| 32 | 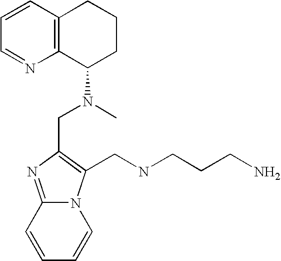 | B |
| 33 | 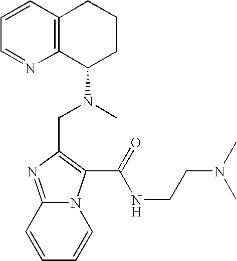 | C |
| 34 | 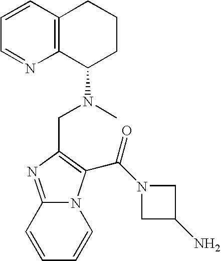 | C |
| 35 | 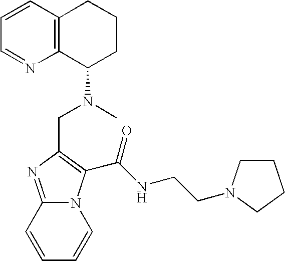 | C |
| 36 | 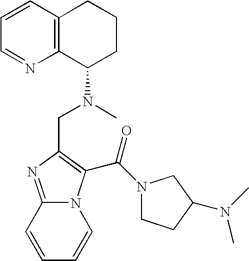 | B |
| 37 | 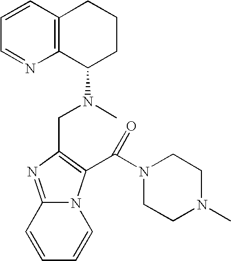 | C |
| 38 | 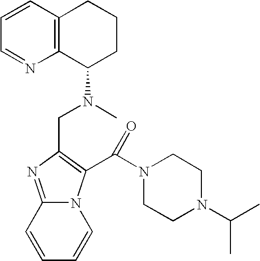 | C |

*“A” indicates an activity level of less than 100 nM in the HIV infectivity assay.
“B” indicates an activity level of between 100 nM to 500 nM in the HIV infectivity assay.
”C” indicates an activity level of between 500 nM and 10 μM in the HIV infectivity assay.

Compounds of the present invention demonstrate anti-HIV activity in the range of IC₅₀ of about 1 nM to about 50 μM. In one aspect of the invention, compounds of the present invention have anti-HIV activity in the range of up to about 100 nM. In another aspect of the invention, compounds of the present invention have anti-HIV activity in the range of from about 100 nM to about 500 nM. In another aspect of the invention, compounds of the present invention have anti-HIV activity in the range of from about 500 nM to 10 μM. In another aspect of the invention, compounds have anti-HIV activity in the range of from about 10 μM to about 50 μM. Moreover, compounds of the present invention are believed to provide a desired pharamcokinetic profile. Also, compounds of the present invention are believed to provide a desired selectivity, such as specificity between toxicity and activity.

Test compounds were employed in free or salt form.

Although specific embodiments of the present invention are herein illustrated and described in detail, the invention is not limited thereto. The above detailed descriptions are provided as exemplary of the present invention and should not be construed as constituting any limitation of the invention. Modifications will be obvious to those skilled in the art, and all modifications that do not depart from the spirit of the invention are intended to be included with the scope of the appended claims.

What is claimed is:

1. A compound of formula (I) 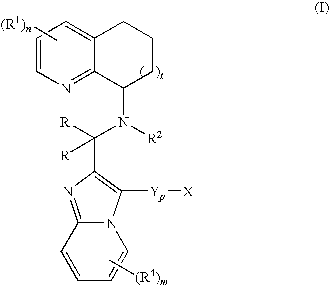
wherein t is 0, 1, or 2;
each R independently is H, C₁-C₈ alkyl, C₂-C₆ alkenyl, C₂-C₆ alkynyl, C₁-C₈ haloalkyl, C₃-C₈ cycloalkyl, —R^{a}Ay, —R^{a}OR¹⁰—R^{a}N(R¹⁰)₂ or —R^{a}S(O)_{q}R¹⁰;
each R¹ independently is halogen, C₁-C₈ haloalkyl, C₁-C₈ alkyl, C₂-C₆ alkenyl, C₂-C₆ alkynyl, C₃-C₈ cycloalkyl, C₃-C₈ cycloalkenyl, -Ay, —N(H)Ay, -Het, —N(H)Het, —OR¹⁰, —OHet, —R^{a}OR¹⁰, —N(R⁶)R⁷, —R^{a}N(R⁶)R⁷, —R^{a}C(O)R¹⁰, —C(O)R¹⁰, —CO₂R¹⁰, —R^{a}CO₂R¹⁰, —C(O)N(R⁶)R⁷, —C(O)Ay, —C(O)Het, —S(O)₂N(R⁶)R⁷, —S(O)_{q}R¹⁰, cyano, nitro, or azido;
n is 0, 1, or 2;
R² is H, C₁-C₈ alkyl, C₁-C₈ haloalkyl, C₃-C₈ cycloalkyl, C₂-C₆ alkenyl, C₂-C₆ alkynyl, —R^{a}cycloalkyl, —R^{a}Ay, —R^{a}OR¹⁰, or —R^{a}S(O)_{q}R¹⁰, wherein R² is not amine or alkylamine, or substituted with amine or alkylamine;
p is 0 or 1;
Y is —NR¹⁰—, —O—, —C(O)N(R¹⁰)—, —N(R¹⁰)C(O)—, —C(O)—, —C(O)O—, —NR¹⁰C(O)N(R¹⁰), —S(O)_{q}—, —S(O)_{q}N(R¹⁰)—, or —N(R¹⁰)S(O)_{q}—;
X is —R^{a}N(R⁶)R⁷, -Ay[N(R⁶)R⁷]_{w}, —R^{a}Ay[N(R⁶)R⁷]_{w}, -Ay[R^{a}N(R⁶)R⁷]_{w}, —R^{a}Ay[R^{a}N(R⁶)R⁷]_{w}, -Het, —R^{a}Het, Het[N(R⁶)R⁷]_{w}, —R^{a}Het[N(R⁶)R⁷]_{w}, -Het[R^{a}N(R⁶)R⁷]_{w}, —R^{a}Het[R^{a}N(R⁶)R⁷]_{w}, -HetR^{a}Ay, or -HetR^{a}Het provided that when p is 0, m is 1 or 2, and X is —R^{a}N(R⁶)R⁷, then R^{a} is not methylene (—CH₂—);
each R^{a} independently is C₁-C₈ alkylene, C₃-C₈ cycloalkylene, C₂-C₆ alkenylene, C₃-C₈ cycloalkenylene, or C₂-C₆ alkynylene, optionally substituted with one or more C₁-C₈ alkyl, hydroxyl or oxo;
each R⁴ independently is halogen, C₁-C₈ haloalkyl, C₁-C₈ alkyl, C₂-C₆ alkenyl, C₂-C₆ alkynyl, C₃-C₈ cycloalkyl, C₃-C₈cycloalkenyl, -Ay, —N(H)Ay, -Het, —N(H)Het, —OR¹⁰, —OHet, —R^{a}OR¹⁰, —N(R⁶)R⁷, —R^{a}N(R⁶)R⁷, —R^{a}C(O)R¹⁰, —C(O)R¹⁰, —CO₂R¹⁰, —R^{a}CO₂R¹⁰, —C(O)N(R⁶)R⁷, —C(O)Ay, —C(O)Het, —S(O)₂N(R⁶)R⁷, —S(O)_{q}R¹⁰, —S(O)_{q}Het, cyano, nitro, or azido;
m is 0
each of R⁶ and R⁷ independently are selected from H, C₁-C₈ alkyl, C₁-C₈ alkenyl, C₂-C₆ alkynyl, C₃-C₈ cycloalkyl, C₃-C₈ cycloalkenyl, —R^{a}cycloalkyl, —R^{a}OR¹⁰, —R^{a}N(R⁸)R⁹, -Ay, -Het, —R^{a}Ay or —R^{a}Het;
each of R⁸ and R⁹ independently are selected from H or C₁-C₈ alkyl;
each R¹⁰ independently is H, C₁-C₈ alkyl, C₃-C₈ cycloalkyl, C₂-C₆ alkenyl, C₂-C₆ alkynyl, or -Ay;
each q independently is 0, 1, or 2;
each w independently is 1 or 2;
each Ay independently represents a C₃-C₁₀ aryl group optionally substituted with one or more of C₁-C₈ alkyl, C₂-C₆ alkenyl, C₂-C₆ alkynyl, C₁-C₈ alkoxy, hydroxyl, halogen, C₁-C₈ haloalkyl, C₃-C₈ cycloalkyl, C₃-C₈ cycloalkoxy, cyano, amide, amino, and C₁-C₈ alkylamino; and
each Het independently represents a C₃-C₇ heterocyclyl or heteroaryl group optionally substituted with one or more of C₁-C₈ alkyl, C₂-C₆ alkenyl, C₂-C₆ alkynyl, C₁-C₈ alkoxy, hydroxyl, halogen, C₁-C₈ haloalkyl, C₃-C₈ cycloalkyl, C₃-C₈ cycloalkoxy, cyano, amide, amino, and C₁-C₈ alkylamino; or pharmaceutically acceptable derivatives thereof.

2. The compound of claim 1 wherein t is 1.

3. The compound of claim 1 wherein t is 2.

4. A compound of formula (I) 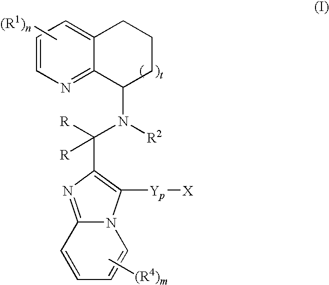
wherein t is 0, 1, or 2;
each R independently is H, C₁-C₈ alkyl, C₂-C₆ alkenyl, C₂-C₆ alkynyl, C₁-C₈ haloalkyl, C₃-C₈ cycloalkyl, —R^{a}Ay, —R^{a}OR¹⁰, —R^{a}N(R¹⁰)₂ or —R^{a}S(O)_{q}R¹⁰,
each R¹ independently is halogen, C₁-C₈ haloalkyl, C₁-C₈ alkyl, C₂-C₆ alkenyl, C₂-C₆ alkynyl, C₃-C₈ cycloalkyl, C₃-C₈ cycloalkenyl, -Ay, —N(H)Ay, -Het, —N(H)Het, —OR¹⁰, —OHet, —R^{a}OR¹⁰, —N(R⁶)R⁷, —R^{a}N(R⁶)R⁷, —R^{a}C(O)R¹⁰, —C(O)R¹⁰, —CO₂R¹⁰, —R^{a}CO₂R¹⁰, —C(O)N(R⁶)R⁷, —C(O)Ay, —C(O)Het, —S(O)₂N(R⁶)R⁷, —S(O)_{q}R¹⁰, cyano, nitro, or azido;
n is 0, 1, or 2;
R² is H, C₁-C₈ alkyl, C₁-C₈ haloalkyl, C₃-C₈ cycloalkyl, C₂-C₆ alkenyl, C₂-C₆ alkynyl, —R^{a}cycloalkyl, —R^{a}Ay, —R^{a}OR¹⁰, or —R^{a}S(O)_{q}R¹⁰, wherein R² is not amine or alkylamine, or substituted with amine or alkylamine;
p is 0 or 1;
Y is —NR¹⁰—, —O—, —C(O)N(R¹⁰)—, —N(R¹⁰)C(O)—, —C(O)—, —C(O)O—, —NR¹⁰C(O)N(R¹⁰)—, —S(O)_{q}—, —S(O)_{q}N(R¹⁰)—, or —N(R¹⁰)S(O)_{q}—;
X is —R^{a}N(R⁶)R⁷, -Ay[N(R⁶)R⁷]_{w}, -Ay[R^{a}N(R⁶)R⁷]_{w}, —R^{a}Ay[R^{a}N(R⁶)R⁷]_{w}, -Het, —R^{a}Het, Het[N(R⁶)R⁷]_{w}, —R^{a}Het[N(R⁶)R⁷]_{w}, -Het[R^{a}N(R⁶)R⁷]_{w}, —R^{a}Het[R^{a}N(R⁶)R⁷]_{w}, -HetR^{a}Ay, or -HetR^{a}Het provided that when p is 0, m is 1 or 2, and X is —R^{a}N(R⁶)R⁷, then R^{a} is not methylene (—CH₂—);
each R^{a} independently is C₁-C₈ alkylene, C₃-C₈ cycloalkylene, C₂-C₆ alkenylene, C₃-C₈ cycloalkenylene, or C₂-C₆ alkynylene, optionally substituted with one or more C₁-C₈ alkyl, hydroxyl or oxo;
each R⁴ independently is halogen, C₁-C₈ haloalkyl, C₁-C₈ alkyl, C₂-C₆ alkenyl, C₂-C₆ alkynyl, C₃-C₈ cycloalkyl, C₃-C₈cycloalkenyl, -Ay, —N(H)Ay, -Het, —N(H)Het, —OR¹⁰, —OHet, —R^{a}OR¹⁰, —N(R⁶)R⁷, —R^{a}N(R⁶)R⁷, —R^{a}C(O)R¹⁰, —C(O)R¹⁰, —CO₂R¹⁰, —R^{a}CO₂R¹⁰, —C(O)N(R⁶)R⁷, —C(O)Ay, —C(O)Het, —S(O)₂N(R⁶)R⁷, —S(O)_{q}R¹⁰, —S(O)_{q}Het, cyano, nitro, or azido;
m is 1 or 2;
each of R⁶ and R⁷ independently are selected from H, C₁-C₈ alkyl, C₁-C₈ alkenyl, C₂-C₆ alkynyl, C₃-C₈ cycloalkyl, C₃-C₈ cycloalkenyl, —R^{a}cycloalkyl, —R^{a}OR¹⁰, —R^{a}N(R⁸)R⁹, -Ay, -Het, —R^{a}Ay or —R^{a}Het;
each of R⁸ and R⁹ independently are selected from H or C₁-C₈ alkyl;
each R¹⁰ independently is H, C₁-C₈ alkyl, C₃-C₈ cycloalkyl, C₂-C₆ alkenyl, C₂-C₆ alkynyl, or -Ay;
each q independently is 0, 1, or 2;
each w independently is 1 or 2;
each Ay independently represents a C₃-C₁₀ aryl group optionally substituted with one or more of C₁-C₈ alkyl, C₂-C₆ alkenyl, C₂-C₆ alkynyl, C₁-C₈ alkoxy, hydroxyl, halogen, C₁-C₈ haloalkyl, C₃-C₈ cycloalkyl, C₃-C₈ cycloalkoxy, cyano, amide, amino, and C₁-C₈ alkylamino; and
each Het independently represents a C₃-C₇ heterocyclyl or heteroaryl group optionally substituted with one or more of C₁-C₈ alkyl, C₂-C₆ alkenyl, C₂-C₆ alkynyl, C₁-C₈ alkoxy, hydroxyl, halogen, C₁-C₈ haloalkyl, C₃-C₈ cycloalkyl, C₃-C₈ cycloalkoxy, cyano, amide, amino, and C₁-C₈ alkylamino; or pharmaceutically acceptable derivatives thereof.

5. The compound of claim 4 wherein m is 1 and R⁴ is —OR¹⁶, -Het, —N(H)Het, —OHet, or —R^{a}N(R⁶)R⁷.

6. The compound of claim 4 wherein m is 1 and R⁴ is —OR¹⁰, C₁-C₈ alkyl, or —N(R⁶)R⁷.

7. The compound of claim 1 wherein p is 0 and X is —R^{a}N(R⁶)R⁷, -Ay[R^{a}N(R⁶)R⁷]_{w}, -Het, —R^{a}Het, Het[N(R⁶)R⁷]_{w}, —R^{a}Het[N(R⁶)R⁷]_{w}, -Het[R^{a}N(R⁶)R⁷]_{w}, —R^{a}Het[R^{a}N(R⁶)R⁷]_{w}.

8. The compound of claim 1 X is —R^{a}N(R⁶)R⁷, -Het, —R^{a}Het, Het[N(R⁶)R⁷]_{w}, or —R^{a}Het[N(R⁶)R⁷]_{w}.

9. The compound of claim 1 wherein X is —R^{a}N(R⁶)R⁷, -Het, or —R^{a}Het.

10. The compound of claim 1 wherein p is 1; Y is —NR¹⁰—, —O—, —S—, —C(O)NR¹⁰—, —N(R¹⁰)CO—, or —S(O)_{q}N(R¹⁰)—; and X is —R^{a}(N(R⁶)R⁷), -Ay[N(R⁶)R⁷]_{w}, —R^{a}Ay[N(R⁶)R⁷]_{w}, -Ay[R^{a}N(R⁶)R⁷]_{w}, —R^{a}Ay[R^{a}N(R⁶)R⁷]_{w}, -Het, —R^{a}Het, Het[N(R⁶)R⁷]_{w}, —R^{a}Het[N(R⁶)R⁷]_{w}, -Het[R^{a}N(R⁶)R⁷]_{w}, —R^{a}Het[R^{a}N(R⁶)R⁷]_{w}, -HetR^{a}Ay, or -HetR^{a}Het.

11. The compound of claim 1 wherein p is 1, Y is —NR¹⁰—, —O—, —C(O)N(R¹⁰), —N(R¹⁰)CO— and X is —R^{a}N(R⁶)R⁷, -Het, —R^{a}Het, Het[N(R⁶)R⁷]_{w}.

12. The compound of claim 1 wherein t is 1 or 2; R is H or C₁-C₈ alkyl; R² is C₁-C₈ alkyl, C₃-C₈ cycloalkyl, or —R^{a}cycloalkyl; n is 0; p is 0 and X is —R^{a}N(R⁶)R⁷, -Het, —R^{a}Het, or Het[N(R⁶)R⁷]_{w}, —R⁶ and R⁷ is H or C₁-C₈ alkyl and -Het is unsubstituted or substituted with C₁-C₈ alkyl or C₃-C₈ cycloalkyl.

13. The compound of claim 4 wherein t is 1 or 2; R is H or C₁-C₈ alkyl; R² is C₁-C₈ alkyl, C₃-C₈ cycloalkyl, or —R^{a}cycloalkyl; n is 0; m is 1 and R⁴ is —OR¹⁰, —N(R⁶)R⁷, Het or N(H)Het; p is 0 and X is —R^{a}N(R⁶)R⁷ provided that R^{a} is not methylene, -Het, —R^{a}Het, or Het[N(R⁶)R⁷]_{w}, —R⁶ and R⁷ is H or C₁-C₈ alkyl and -Het is unsubstituted or substituted with C₁-C₈ alkyl or C₃-C₈ cycloalkyl.

14. The compound of claim 1 wherein t is 1 or 2; R is H or C₁-C₈ alkyl; R² is C₁-C₈ alkyl, C₃-C₈ cycloalkyl, or —R^{a}cycloalkyl; n is 0; p is 1; Y is —N(R¹⁰)—, —O—, —C(O)N(R¹⁰)—, or —N(R¹⁰)CO—; X is —R^{a}N(R⁶)R⁷, -Het, —R^{a}Het, or Het[N(R⁶)R⁷]_{w}, and R⁶ and R⁷ is H or C₁-C₈ alkyl and Het is unsubstituted or substituted with C₁-C₈ alkyl or C₃-C₈ cycloalkyl.

15. The compound of claim 4 wherein t is 1 or 2; R is H or C₁-C₈ alkyl; R² is C₁-C₈ alkyl, C₃-C₈ cycloalkyl, or —R^{a}cycloalkyl; n is 0; m is 1 and R⁴ is —OR¹⁰, —N(R⁶)R⁷, Het or N(H)Het; p is 1; Y is —N(R¹⁰)—, —O—, —C(O)N(R¹⁰)—, or —N(R¹⁰)CO—; X is —R^{a}N(R⁶)R⁷ provided that R^{a} is not methylene, -Het, —R^{a}Het, or Het[N(R⁶)R⁷]_{w}, and R⁶ and R⁷ is H or C₁-C₈ alkyl and Het is unsubstituted or substituted with C₁-C₈ alkyl or C₃-C₈ cycloalkyl.

16. The compound of claim 1 wherein the substituent R⁴ is located on the depicted benzimidazole ring as in formula (I-A): 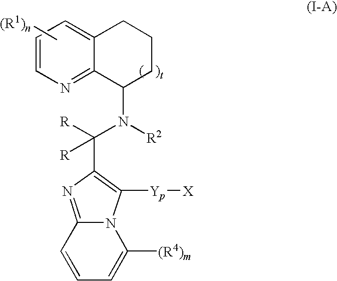
or pharmaceutically acceptable derivatives thereof.

17. The compound of claim 1 wherein Het is piperidine, piperazine, azetidine, pyrrolidine, imidazole, or pyridine.

18. The compound of claim 1 wherein -Het is unsubstituted or substituted with one or more C₁-C₈ alkyl or C₃-C₈ cycloalkyl.

19. A compound selected from the group consisting of
(8S)-N-Methyl-N-{[3-(4-pyridinyl)imidazo[1,2-a]pyridin-2-yl]methyl}-5,6,7,8-tetrahydro-8-quinolinamine;
(8S)-N-{[3-(3-Aminopropyl)imidazo[1,2-a]pyridin-2-yl]methyl}-N-methyl-5,6,7,8-tetrahydro-8-quinolinamine;
(8S)-N-({3-[3-(Dimethylamino)propyl]imidazo[1,2-a]pyridin-2-yl}methyl)-N-methyl-5,6,7,8-tetrahydro-8-quinolinamine;
(8S)-N-Methyl-N-[(3-{3-[(2-methylpropyl)amino]propyl}imidazo[1,2-a]pyridin-2-yl)methyl]-5,6,7,8-tetrahydro-8-quinolinamine;
(8S)-N-({3-[(Dimethylamino)methyl]imidazo[1,2-a]pyridin-2-yl}methyl)-N-methyl-5,6,7,8-tetrahydro-8-quinolinamine;
N²,N²-Dimethyl-N¹-{[2-({methyl[(8S)-5,6,7,8-tetrahydro-8-quinolinyl]amino}methyl)imidazo[1,2-a]pyridin-3-yl]methyl}glycinamide;
(8S)-N-Methyl-N-{[3-(1-pyrrolidinylmethyl)imidazo[1,2-a]pyridin-2-yl]methyl}-5,6,7,8-tetrahydro-8-quinolinamine;
(8S)-N-({3-[6-(Dimethylamino)-3-pyridinyl]imidazo[1,2-a]pyridin-2-yl}methyl)-N-methyl-5,6,7,8-tetrahydro-8-quinolinamine;
(8S)-N-Methyl-N-{[3-(1-piperidinylmethyl)imidazo[1,2-a]pyridin-2-yl]methyl}-5,6,7,8-tetrahydro-8-quinolinamine;
(8S)-N-[(3-{[3-(Dimethylamino)-1-pyrrolidinyl]methyl}imidazo[1,2-a]pyridin-2-yl)methyl]-N-methyl-5,6,7,8-tetrahydro-8-quinolinamine;
(8S)-N-Methyl-N-({3-[6-(4-methyl-1-piperazinyl)-3-pyridinyl]imidazo[1,2-a]pyridin-2-yl}methyl)-5,6,7,8-tetrahydro-8-quinolinamine;
N,N,N′-Trimethyl-N′-{[2-({methyl[(8S)-5,6,7,8-tetrahydro-8-quinolinyl]amino}methyl)imidazo[1,2-a]pyridin-3-yl]methyl}-1,2-ethanediamine;
(8S)-N-Methyl-N-[(3-{[methyl(1-methylethyl)amino]methyl}imidazo[1,2-a]pyridin-2-yl)methyl]-5,6,7,8-tetrahydro-8-quinolinamine;
(8S)-N-Methyl-N-[(3-{[methyl(1-methyl-3-pyrrolidinyl)amino]methyl}imidazo[1,2-a]pyridin-2-yl)methyl]-5,6,7,8-tetrahydro-8-quinolinamine;
(8S)-N-Methyl-N-[(3-{[methyl(1-methyl-4-piperidinyl)amino]methyl}imidazo[1,2-a]pyridin-2-yl)methyl]-5,6,7,8-tetrahydro-8-quinolinamine;
(Methyl{[2-({methyl[(8S)-5,6,7,8-tetrahydro-8-quinolinyl]amino}methyl)imidazo[1,2-a]pyridin-3-yl]methyl}amino)acetonitrile;
3-(Methyl{[2-({methyl[(8S)-5,6,7,8-tetrahydro-8-quinolinyl]amino}methyl)imidazo[1,2-a]pyridin-3-yl]methyl}amino)propanenitrile;
(8S)-N-Methyl-N-{[3-(3-pyridinyl)imidazo[1,2-a]pyridin-2-yl]methyl}-5,6,7,8-tetrahydro-8-quinolinamine;
(8S)-N-Methyl-N-({3-[6-(4-morpholinyl)-3-pyridinyl]imidazo[1,2-a]pyridin-2-yl}methyl)-5,6,7,8-tetrahydro-8-quinolinamine;
(8S)-N-Methyl-N-{[3-(4-morpholinylmethyl)imidazo[1,2-a]pyridin-2-yl]methyl}-5,6,7,8-tetrahydro-8-quinolinamine;
(8S)-N-Methyl-N-({3-[6-(1-pyrrolidinyl)-3-pyridinyl]imidazo[1,2-a]pyridin-2-yl}methyl)-5,6,7,8-tetrahydro-8-quinolinamine;
(8S)-N-{[3-({Bis[2-(methyloxy)ethyl]amino}methyl)imidazo[1,2-a]pyridin-2-yl]methyl}-N-methyl-5,6,7,8-tetrahydro-8-quinolinamine;
(8S)-N-({3-[(Diethylamino)methyl]imidazo[1,2-a]pyridin-2-yl}methyl)-N-methyl-5,6,7,8-tetrahydro-8-quinolinamine;
(8S)-N-Methyl-N-{[3-({methyl[2-(methyloxy)ethyl]amino}methyl)imidazo[1,2-a]pyridin-2-yl]methyl}-5,6,7,8-tetrahydro-8-quinolinamine;
(8S)-N-Methyl-N-[(3-{[methyl(2-methylpropyl)amino]methyl}imidazo[1,2-a]pyridin-2-yl)methyl]-5,6,7,8-tetrahydro-8-quinolinamine;
(8S)-N-({3-[2-(Dimethylamino)-4-pyridinyl]imidazo[1,2-a]pyridin-2-yl}methyl)-N-methyl-5,6,7,8-tetrahydro-8-quinolinamine;
(8S)-N-Methyl-N-({3-[2-(4-morpholinyl)-4-pyridinyl]imidazo[1,2-a]pyridin-2-yl}methyl)-5,6,7,8-tetrahydro-8-quinolinamine;
N,N-dimethyl-N′-[(2-{[methyl(5,6,7,8-tetrahydro-8-quinolinyl)amino]methyl}imidazo[1,2-a]pyridin-3-yl)methyl]-1,2-ethanediamine;
N-methyl-N-({3-[(4-methyl-1-piperazinyl)methyl]imidazo[1,2-a]pyridin-2-yl}methyl)-5,6,7,8-tetrahydro-8-quinolinamine;
N-[(2-{[methyl(5,6,7,8-tetrahydro-8-quinolinyl)amino]methyl}imidazo[1,2-a]pyridin-3-yl)methyl]-1,3-propanediamine;
N-[(3-{[3-(dimethylamino)-1-pyrrolidinyl]carbonyl}imidazo[1,2-a]pyridin-2-yl)methyl]-N-methyl-5,6,7,8-tetrahydro-8-quinolinamine;
N,N,N′-trimethyl-N′-{5-[2-({methyl[(8S)-5,6,7,8-tetrahydro-8-quinolinyl]amino}methyl)imidazo[1,2-a]pyridin-3-yl]-2-pyridinyl}-1,2-ethanediamine;
(8S)-N-[(3-{6-[3-(Dimethylamino)-1-pyrrolidinyl]-3-pyridinyl}imidazo[1,2-a]pyridin-2-yl)methyl]-N-methyl-5,6,7,8-tetrahydro-8-quinolinamine;
N-[2-(dimethylamino)ethyl]-2-{[methyl(5,6,7,8-tetrahydro-8-quinolinyl)amino]methyl}imidazo[1,2-a]pyridine-3-carboxamide;
N-({3-[(3-amino-1-azetidinyl)carbonyl]imidazo[1,2-a]pyridin-2-yl}methyl)-N-methyl-5,6,7,8-tetrahydro-8-quinolinamine;
2-{[methyl(5,6,7,8-tetrahydro-8-quinolinyl)amino]methyl}-N-[2-(1-pyrrolidinyl)ethyl]imidazo[1,2-a]pyridine-3-carboxamide;
N-methyl-N-({3-[(4-methyl-1-piperazinyl)carbonyl]imidazo[1,2-a]pyridin-2-yl}methyl)-5,6,7,8-tetrahydro-8-quinolinamine;
N-methyl-N-[(3-{[4-(1-methylethyl)-1-piperazinyl]carbonyl}imidazo[1,2-a]pyridin-2-yl)methyl]-5,6,7,8-tetrahydro-8-quinolinamine; and pharmaceutically acceptable derivatives thereof.

20. A compound selected from the group consisting of:
(8S)-N-Methyl-N-{[3-(4-pyridinyl)imidazo[1,2-a]pyridin-2-yl]methyl}-5,6,7,8-tetrahydro-8-quinolinamine;
(8S)-N-{[3-(3-Aminopropyl)imidazo[1,2-a]pyridin-2-yl]methyl}-N-methyl-5,6,7,8-tetrahydro-8-quinolinamine;
(8S)-N-({3-[3-(Dimethylamino)propyl]imidazo[1,2-a]pyridin-2-yl}methyl)-N-methyl-5,6,7,8-tetrahydro-8-quinolinamine;
(8S)-N-Methyl-N-[(3-{3-[(2-methylpropyl)amino]propyl}imidazo[1,2-a]pyridin-2-yl)methyl]-5,6,7,8-tetrahydro-8-quinolinamine;
(8S)-N-({3-[(Dimethylamino)methyl]imidazo[1,2-a]pyridin-2-yl}methyl)-N-methyl-5,6,7,8-tetrahydro-8-quinolinamine;
N²,N²-Dimethyl-N¹-{[2-({methyl[(8S)-5,6,7,8-tetrahydro-8-quinolinyl]amino}methyl)imidazo[1,2-a]pyridin-3-yl]methyl}glycinamide;
(8S)-N-Methyl-N-{[3-(1-pyrrolidinylmethyl)imidazo[1,2-a]pyridin-2-yl]methyl}-5,6,7,8-tetrahydro-8-quinolinamine;
(8S)-N-({3-[6-(Dimethylamino)-3-pyridinyl]imidazo[1,2-a]pyridin-2-yl}methyl)-N-methyl-5,6,7,8-tetrahydro-8-quinolinamine;
(8S)-N-Methyl-N-{[3-(1-piperidinylmethyl)imidazo[1,2-a]pyridin-2-yl]methyl}-5,6,7,8-tetrahydro-8-quinolinamine;
(8S)-N-[(3-{[3-(Dimethylamino)-1-pyrrolidinyl]methyl}imidazo[1,2-a]pyridin-2-yl)methyl]-N-methyl-5,6,7,8-tetrahydro-8-quinolinamine;
(8S)-N-Methyl-N-({3-[6-(4-methyl-1-piperazinyl)-3-pyridinyl]imidazo[1,2-a]pyridin-2-yl}methyl)-5,6,7,8-tetrahydro-8-quinolinamine;
N,N,N′-Trimethyl-N′-{[2-({methyl[(8S)-5,6,7,8-tetrahydro-8-quinolinyl]amino}methyl)imidazo[1,2-a]pyridin-3-yl]methyl}-1,2-ethanediamine;
(8S)-N-Methyl-N-[(3-{[methyl(1-methylethyl)amino]methyl}imidazo[1,2-a]pyridin-2-yl)methyl]-5,6,7,8-tetrahydro-8-quinolinamine;
(8S)-N-Methyl-N-[(3-{[methyl(1-methyl-3-pyrrolidinyl)amino]methyl}imidazo[1,2-a]pyridin-2-yl)methyl]-5,6,7,8-tetrahydro-8-quinolinamine;
(8S)-N-Methyl-N-[(3-{[methyl(1-methyl-4-piperidinyl)amino]methyl}imidazo[1,2-a]pyridin-2-yl)methyl]-5,6,7,8-tetrahydro-8-quinolinamine;
(Methyl{[2-({methyl[(8S)-5,6,7,8-tetrahydro-8-quinolinyl]amino}methyl)imidazo[1,2-a]pyridin-3-yl]methyl}amino)acetonitrile;
3-(Methyl{[2-({methyl[(8S)-5,6,7,8-tetrahydro-8-quinolinyl]amino}methyl)imidazo[1,2-a]pyridin-3-yl]methyl}amino)propanenitrile;
and pharmaceutically acceptable derivatives thereof.

21. (canceled)

22. A pharmaceutical composition comprising a compound according to claim 1, and a pharmaceutically acceptable carrier.

23. A pharmaceutical composition according to claim 22 in the form of a tablet or capsule.

24. A pharmaceutical composition according to claim 22 in the form of a liquid or suspension.

25-42. (canceled)
